(19) United States
(12) Patent Application Publication
(10) Pub. No.: US 2024/0035004 A1
(43) Pub. Date: Feb. 1, 2024

(54) VARIANT SUCROSE PERMEASE POLYPEPTIDES

(71) Applicant: INBIOSE N.V., Zwijnaarde (BE)

(72) Inventors: Joeri Beauprez, Zwijnaarde (BE); Katarzyna Ciesielska, Zwijnaarde (BE); Nausicaä Lannoo, Zwijnaarde (BE); Kristof Vandewalle, Zwijnaarde (BE); Annelies Vercauteren, Zwijnaarde (BE)

(21) Appl. No.: 18/254,345

(22) PCT Filed: Dec. 17, 2021
(86) PCT No.: PCT/EP2021/086396

§ 371 Date: May 24, 2023

(30) Foreign Application Priority Data
Dec. 18, 2020 (EP) 20215612.1

(51) Int. Cl. C12N 9/12; C12N 1/20

(52) U.S. Cl. CPC C12N 9/1205; C12N 1/205; C12Y 207/01; C12R 2001/19

(57) ABSTRACT

The present invention is in the technical field of synthetic biology and metabolic engineering. More particularly, the present invention is in the technical field of fermentation of metabolically engineered cells. The present invention describes new sucrose permease polypeptides, and their applications. The present invention also describes a metabolically engineered cell for the production of a glycosylated product using the novel sucrose permease polypeptides. Furthermore, the present invention provides a method for the production of a glycosylated product by a cell using the novel sucrose permease polypeptides as well as the purification of said glycosylated product from the cultivation.

### FIELD OF THE INVENTION

The present invention is in the technical field of synthetic biology and metabolic engineering. More particularly, the present invention is in the technical field of fermentation of metabolically engineered cells. The present invention describes new sucrose permease polypeptides, and their applications. The present invention also describes a metabolically engineered cell for the production of a glycosylated product using the novel sucrose permease polypeptides. Furthermore, the present invention provides a method for the production of a glycosylated product by a cell using the novel sucrose permease polypeptides as well as the purification of said glycosylated product from the cultivation.

### BACKGROUND

Sucrose is an important carbon source for bio-based fermentation. It is a disaccharide composed of a glucose unit linked to a fructose unit by a glycosidic bond. Sucrose (mostly obtained from sugarcane juice or sugar beet) has economic and environmental advantages over glucose (typically from corn) with respect to price, energy input, greenhouse gas emission and acidification making it a cost-competitive carbon source for industrial bioprocesses (Bruschi et al., Biotech. Adv. 2012, 30:1001; Sabri et al., Appl. Environ. Microbiol. 2013, 79:478). There are two groups of metabolic pathways for sucrose utilization based on two different sugar transport mechanisms: the phosphoenolpyruvate (PEP)-dependent phosphotransferase system (PTS) and the non-PTS system (Feng et al., Microb. Cell Fact. 2017, 16:98). In the PTS associated system, extracellular sucrose is transported across the cytoplasmic membrane into the cell via a sucrose-specific PEP-dependent phosphotransferase and concomitantly converted to sucrose-6-phosphate (sucrose-6-P). Sucrose-6-P is then hydrolyzed in the cell by a sucrose-6-P hydrolase (invertase) generating fructose and glucose-6-phosphate (glucose-6-P). In the non-PTS system, extracellular sucrose is taken up in the cell by a sucrose permease and then inside the cell either hydrolyzed by sucrase into fructose and glucose or converted by a sucrose phosphorylase in the presence of inorganic phosphate to yield fructose and glucose-1-phosphate (glucose-1-P). Fructose is then catalysed by an ATP-dependent fructokinase to generate fructose-6-P while glucose is catalysed by an ATP-dependent glucokinase to generate glucose-6-P. Glucose-1-P can be converted to glucose-6-P by a phosphoglucomutase. Finally, both glucose-6-P and fructose-6-P are then entering the glycolytic pathway.

Although sucrose is an attractive carbon source, the ability to utilize sucrose as a sole carbon source is a highly variable phenotype among enteric bacteria. Most industrial microbial strains cannot naturally utilize sucrose and need engineering enabling the strains to use sucrose. In contrast to E. coli K-12, the E. coli W strain and the E. coli wild-type isolate EC3132 are capable of utilizing sucrose, yet at slow rates as compared to growth on glucose. These strains contain the cscA, cscK and cscB genes that encode an invertase, a fructokinase and a sucrose:H+ symporter (sucrose permease), respectively. These genes are negatively controlled at the transcriptional level by the repressor CscR (Jahreis et al, J. Bacteriol. 2002, 184:5307). The cscBKA or cscBKAR gene clusters have already been used as transferable non-PTS sucrose utilization cassettes to develop sucrose-utilizing strains that do not have innate sucrose-utilizing ability for the production of for example 1,4-butanediol, glycerol and glycerol-derived products, L-fucose, succinic acid, lactic acid, L-amino acids (EP3050970, U.S. Pat. No. 8,686,114, US20140024087, US20170137829, WO2016120448, Bruschi et al., Biotech. Adv. 2012, 30:1001; Lee et al., Appl. Microbiol. Biotechnol. 2010, 88:905). Nevertheless, the challenge remains to obtain industrial strains with high ability to use sucrose inside the cell, high growth rates on sucrose and/or high productivity of the product of interest. It is an object of the present invention to provide for tools and methods by means of which a glycosylated product can be produced by a sucrose-utilizing cell in an efficient, time and cost-effective way and if needed, continuous process. According to the invention, this and other objects are achieved by providing a cell and a method and novel sucrose permease polypeptides for the production of a glycosylated product.

### DESCRIPTION

### Summary of the Invention

The present invention provides novel sucrose permease polypeptides that have distinct mutations compared to the well-known sucrose permease CscB from the E. coli W strain. The present invention also provides a metabolically engineered cell and a method for the production of a glycosylated product such as a monosaccharide, a phosphorylated monosaccharide, an activated monosaccharide, a disaccharide, an oligosaccharide, a glycoprotein or a glycolipid. The method comprises the steps of providing a cell comprising a pathway for the production of a glycosylated product, which is capable to express one or more of said novel sucrose permease polypeptides and cultivating said cell under conditions permissive to produce said glycosylated product. The present invention also provides methods to separate said glycosylated product.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, purification steps are performed according to the manufacturer's specifications.

In the specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the invention herein and within the scope of this invention, which is limited only by the claims, construed in accordance with the patent law, including the doctrine of equivalents. In the claims which follow, reference characters used to designate claim steps are provided for convenience of description only, and are not intended to imply any particular order for performing the steps.

In this document and in its claims, the verbs “to comprise” and “to have”, and their conjugations are used in their non-limiting sense to mean that items following the word are included, but items not specifically mentioned are not excluded. Throughout the application, the verbs “to comprise” and “to have” may be replaced by “to consist” or “to consist essentially of” and vice versa. In addition the verb “to consist” may be replaced by “to consist essentially of” meaning that a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there is one and only one of the elements. The indefinite article “a” or “an” thus usually means “at least one”. According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

The terms “recombinant” or “transgenic” or “metabolically engineered” or “genetically modified”, as used herein with reference to a cell or host cell are used interchangeably and indicates that the cell replicates a heterologous nucleic acid, or expresses a peptide or protein encoded by a heterologous nucleic acid (i.e., a sequence “foreign to said cell” or a sequence “foreign to said location or environment in said cell”). Such cells are described to be transformed with at least one heterologous or exogenous gene or are described to be transformed by the introduction of at least one heterologous or exogenous gene. Metabolically engineered or recombinant or transgenic cells can contain genes that are not found within the native (non-recombinant) form of the cell. Recombinant cells can also contain genes found in the native form of the cell wherein the genes are re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one which has been produced by a recombinant cell. A “heterologous sequence” or a “heterologous nucleic acid”, as used herein, is one that originates from a source foreign to the particular cell (e.g. from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g. by transfection, transformation, conjugation or transduction, into the genome of the host microorganism cell, wherein techniques may be applied which will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” cell or microorganism as used within the context of the present invention refers to a cell or microorganism which is genetically modified.

The term “endogenous,” within the context of the present invention refers to any polynucleotide, polypeptide or protein sequence which is a natural part of a cell and is occurring at its natural location in the cell chromosome. The term “exogenous” refers to any polynucleotide, polypeptide or protein sequence which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter,” even though the promoter may be derived from the same species (or, in some cases, the same organism) as the gene to which it is linked.

The term “modified activity” of a protein or an enzyme relates to a change in activity of the protein or the enzyme compared to the wild type, i.e. natural, activity of said protein or enzyme. Said modified activity can either be an abolished, impaired, reduced or delayed activity of said protein or enzyme compared to the wild type activity of the protein or the enzyme but can also be an accelerated or an enhanced activity of said protein or the enzyme compared to the wild type activity of the protein or the enzyme. A modified activity of a protein or an enzyme is obtained by modified expression of said protein or enzyme or is obtained by expression of a modified, i.e. mutant form of the protein or enzyme. A modified activity of an enzyme further relates to a modification in the apparent Michaelis constant Km and/or the apparent maximal velocity (Vmax) of the enzyme.

The term “modified expression” of a gene relates to a change in expression compared to the wild type expression of said gene in any phase of the production process of the encoded protein. Said modified expression is either a lower or higher expression compared to the wild type, wherein the term “higher expression” is also defined as “overexpression” of said gene in the case of an endogenous gene or “expression” in the case of a heterologous gene that is not present in the wild type strain. Lower expression or reduced expression is obtained by means of common well-known technologies for a skilled person (such as the usage of siRNA, CrispR, CrispRi, riboswitches, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e. statistically significantly ‘less-able’ compared to a functional wild-type gene) or completely unable (such as knocked-out genes) to produce functional final products. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can for instance be obtained by mutating one or more base pairs in the promoter sequence or changing the promoter sequence fully to a constitutive promoter with a lower expression strength compared to the wild type or an inducible promoter which result in regulated expression or a repressible promoter which results in regulated expression Overexpression or expression is obtained by means of common well-known technologies for a skilled person, wherein said gene is part of an “expression cassette” which relates to any sequence in which a promoter sequence, untranslated region sequence (containing either a ribosome binding sequence or Shine Dalgarno sequence), a coding sequence and optionally a transcription terminator is present, and leading to the expression of a functional active protein. Said expression is either constitutive or regulated.

The term “constitutive expression” is defined as expression that is not regulated by transcription factors other than the subunits of the RNA polymerase holoenzyme (comprising bacterial sigma factors like σ⁷⁰, σ⁵⁴, or related σ-factors and the yeast mitochondrial RNA polymerase specificity factor MTF1 that co-associate with the RNA polymerase core enzyme). The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as a facultative or regulatory or tuneable expression of a gene that is only expressed upon a certain natural condition of the host (e.g. mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminium, copper, zinc, nitrogen, phosphates, xylene, carbon or nitrogen depletion, or substrates or the produced product or chemical repression, as a response to an environmental change (e.g. anaerobic or aerobic growth, oxidative stress, pH shifts, temperature changes like e.g. heat-shock or cold-shock, osmolarity, light conditions, starvation) or dependent on the position of the developmental stage or the cell cycle of said host cell including but not limited to apoptosis and autophagy. Regulated expression allows for control as to when a gene is expressed.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous. The term “wild type” refers to the commonly known genetic or phenotypical situation as it occurs in nature.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions, and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

The term “derivative” of a polypeptide, as used herein, is a polypeptide which may contain deletions, additions or substitutions of amino acid residues within the amino acid sequence of the polypeptide, but which result in a silent change, thus producing a functionally equivalent polypeptide. Amino acid substitutions may be made based on similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Within the context of this invention, a derivative polypeptide as used herein, refers to a polypeptide capable of exhibiting a substantially similar in vivo activity as the original polypeptide as judged by any of a number of criteria, including but not limited to enzymatic activity, and which may be differentially modified during or after translation. Furthermore, non-classical amino acids or chemical amino acid analogues can be introduced as a substitution or addition into the original polypeptide sequence.

In some embodiments, the present invention contemplates making functional variants by modifying the structure of an enzyme as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the invention results in a functional homologue can be readily determined by assessing the ability of the variant polypeptide to produce a response in cells in a fashion similar to the wild-type polypeptide.

The term “functional homologue” as used herein describes those molecules that have sequence similarity and also share at least one functional characteristic such as a biochemical activity. Functional homologues will typically give rise to the same characteristics to a similar, but not necessarily the same, degree. Functionally homologous proteins give the same characteristics where the quantitative measurement produced by one homologue is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homologue will have the above-recited percent enzymatic activities compared to the original enzyme. Where the molecule is a DNA-binding molecule (e.g., a polypeptide) the homologue will have the above-recited percentage of binding affinity as measured by weight of bound molecule compared to the original molecule.

A functional homologue and the reference polypeptide may be naturally occurring polypeptides, and the sequence similarity may be due to convergent or divergent evolutionary events. Functional homologues are sometimes referred to as orthologs, where “ortholog”, refers to a homologous gene or protein that is the functional equivalent of the referenced gene or protein in another species.

Functional homologues can be identified by analysis of nucleotide and polypeptide sequence alignments. For example, performing a query on a database of nucleotide or polypeptide sequences can identify homologues of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment”, with respect to a polynucleotide, refers to a clone or any part of a polynucleotide molecule, particularly a part of a polynucleotide that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, for example at least about 30 nucleotides or at least about 50 nucleotides of any of the sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide.

As such, a fragment of a polypeptide SEQ ID NO preferably means a polypeptide sequence which comprises or consists of an amount of consecutive amino acid residues from said polypeptide SEQ ID NO and wherein said amount of consecutive amino acid residues is preferably at least 50.0%, 60.0%, 70.0%, %, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0% of the full-length of said polypeptide SEQ ID NO and which performs at least one biological function of the intact polypeptide in substantially the same manner, preferably to a similar or greater extent, as does the intact polypeptide which can be routinely assessed by the skilled person. As such, a fragment of a polypeptide SEQ ID NO preferably means a polypeptide sequence which comprises or consists of said polypeptide SEQ ID NO, wherein an amount of consecutive amino acid residues is missing and wherein said amount is no more than 50.0%, 40.0%, 30.0% of the full-length of said polypeptide SEQ ID NO, preferably no more than 20.0%, 15.0%, 10.0%, 9.0, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than most preferably no more than 2.5%, of the full-length of said polypeptide SEQ ID NO and which performs at least one biological function of the intact polypeptide in substantially the same manner, preferably to a similar or greater extent, as does the intact polypeptide which can be routinely assessed by the skilled person.

Fragments may additionally or alternatively include subsequences of polypeptides and protein molecules, or a subsequence of the polypeptide. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example at least about 20 amino acid residues in length, for example at least about 30 amino acid residues in length. Preferentially a fragment is a functional fragment that has at least one property or activity of the polypeptide from which it is derived, such as, for example, the fragment can include a functional domain or conserved domain of a polypeptide.

A domain can be characterized, for example, by a Pfam (El-Gebali et al., Nucleic Acids Res. 47 (2019) D427-D432), an IPR (InterPro domain) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), or a PTHR domain (http://www.pantherdb.org) (Mi et al., Nucleic Acids. Res. 41 (2013) D377-D386; Thomas et al., Genome Research 13 (2003) 2129-2141). The content of protein domain databases is fixed at each release and is not to be changed. When the content of a specific database is changed, this specific database receives a new release version with a new release date. All release versions for each database with their corresponding release dates and specific content as annotated at these specific release dates are available and known to those skilled in the art.

In the present invention, polypeptide sequences are written in the form of a sequence of amino acids in one-letter code. In case an amino acid at a specific place in such polypeptide sequence can be several amino acids, that specific place will have amino acid code a, b, c, d, e or f. Unless otherwise mentioned herein, the letter “a” refers to any amino acid possible excluding histidine whereas the letters “b, c, d, e and f” refer to any amino acid possible. Preferably, “c” refers to any amino acid possible excluding methionine, more preferably “c” refers to an amino acid selected from the list consisting of alanine, cysteine and phenylalanine.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using different algorithms like for example BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle (https://galaxy-iuc.github.io/emboss-5.0-docs/needle.html).

The BLAST (Basic Local Alignment Search Tool)) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: https://blast.ncbi.nlm.nih.gov/Blast.cgi.

Clustal Omega (Clustal Ω) is a multiple sequence alignment program that uses seeded guide trees and HMM profile-profile techniques to generate alignments between three or more sequences. It produces biologically meaningful multiple sequence alignments of divergent sequences. The web interface for Clustal Ω is available at https://www.ebi.ac.uk/Tools/msa/clustalo/. Default parameters for multiple sequence alignments and calculation of percent identity of protein sequences using the Clustal Ω method are: enabling de-alignment of input sequences: FALSE; enabling mbed-like clustering guide-tree: TRUE; enabling mbed-like clustering iteration: TRUE; Number of (combined guide-tree/HMM) iterations: default(0); Max Guide Tree Iterations: default [−1]; Max HMM Iterations: default [−1]; order: aligned. MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g. BLOSUM50, BLOSUM62, and PAM250) to employ with their protein sequence examination. EMBOSS Needle (https://galaxy-iuc.github.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

As used herein, a polypeptide having an amino acid sequence having at least 80% overall sequence identity to the full-length sequence of a reference polypeptide sequence is to be understood as that the sequence has 80%, 81%, 82%, 83%, 84%, 85%, 86%, 87%, 88%, 89%, 90%, 91%, 92%, 93%, 94%, %, 96%, 97%, 98%, 99%, 100% overall sequence identity to the full-length of the amino acid sequence of the reference polypeptide sequence. Throughout the application, unless explicitly specified otherwise, a polypeptide comprising/consisting/having an amino acid sequence having at least 80% sequence overall sequence identity to the full-length amino acid sequence of a reference polypeptide, usually indicated with a SEQ ID NO, preferably has at least 85%, 90%, 91%, 92%, 93%, 94%, 95%, 96%, 97%, 98% or 99%, more preferably has at least 85%, even more preferably has at least 90%, even more preferably has at least 95%, most preferably has at least 97%, overall sequence identity to the full length reference sequence.

For the purpose of this invention, the overall sequence identity of a polypeptide is determined by the program EMBOSS Needle 5.0 (https://galaxy-iuc.github.io/emboss-5.0-docs/needle.html), preferably with default parameters (the substitution matrix EBLOSUM62, the gap opening penalty 10, and the gap extension penalty 0.5) and preferably with sequences of mature proteins (i.e. without taking into account secretion signals or transit peptides).

The term “CscB” as used herein refers to the sucrose permease CscB from E. coli W, having UniProt Identifier EOIXR1 and characterized by the presence of InterPro domains IPR000576, IPR018457, IPR020846 and IPR036259 as present in the InterPro 82.0 database with release date 8 Oct. 2020. Said E. coli CscB is also characterized by the presence of Pfam motif PF01306 as present in the Pfam 33.1 database released on 11 Jun. 2020. The sucrose permease CscB belongs to the oligosaccharide/H(+) symporter subfamily of the major facilitator superfamily (MFS) and catalyses sugar/H(+) symport across the cytoplasmic membrane. In other words, the protein enables the transfer of sucrose from one side of a membrane to the other according to the reaction: sucrose(out)+H+(out)=sucrose(in)+H+(in). Said CscB is also known as the secondary active transporter from E. coli.

The term “sucrose permease” as used herein refers to a protein capable to transfer extracellular sucrose into the cell with the concomitant import (symport) of a proton (H+).

The term “polypeptide having sucrose permease activity” as used herein refers to a polypeptide that is capable of mediating the transport of sucrose into cells.

The terms “nucleotide-activated sugar” or “activated sugar” or “activated monosaccharide” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-Iyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-N-glycolylneuraminic acid (CMP-Neu5Gc), GDP-fucose (GDP-Fuc), GDP-rhamnose and UDP-xylose. Nucleotide-sugars act as glycosyl donors in glycosylation reactions. Glycosylation reactions are reactions that are catalysed by glycosyltransferases.

The term “glycosyltransferase” as used herein refers to an enzyme capable to catalyse the transfer of sugar moieties from activated donor molecules to specific acceptor molecules, forming glycosidic bonds. The as such synthesized oligosaccharides can be of the linear type or of the branched type and can contain multiple monosaccharide building blocks. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997); Coutinho et al. J. Mol. Biol. 328, 307-317 (2003)) and is available on the CAZy (CArbohydrate-Active EnZymes) website (www.cazy.org) (Cantarel et al., Nucleic Acids Res. 37, D233-D238 (2009)), with last update on 20 Mar. 2019.

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases and fucosaminyltransferases.

Fucosyltransferases are glycosyltransferases that transfer a fucose residue (Fuc) from a GDP-fucose (GDP-Fuc) donor onto a glycan acceptor. Fucosyltransferases comprise alpha-1,2-fucosyltransferases, alpha-1,3-fucosyltransferases, alpha-1,4-fucosyltransferases and alpha-1,6-fucosyltransferases that catalyse the transfer of a Fuc residue from GDP-Fuc onto a glycan acceptor via alpha-glycosidic bonds. Fucosyltransferases can be found but are not limited to the GT10, GT11, GT23, GT65 and GT68 CAZy families. Sialyltransferases are glycosyltransferases that transfer a sialyl group (like Neu5Ac or Neu5Gc) from a donor (like CMP-Neu5Ac or CMP-Neu5Gc) onto a glycan acceptor. Sialyltransferases comprise alpha-2,3-sialyltransferases and alpha-2,6-sialyltransferases that catalyse the transfer of a sialyl group onto a glycan acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from an UDP-galactose (UDP-Gal) donor onto a glycan acceptor. Galactosyltransferases comprise beta-1,3-galactosyltransferases, beta-1,4-galactosyltransferases, alpha-1,3-galactosyltransferases and alpha-1,4-galactosyltransferases that transfer a Gal residue from UDP-Gal onto a glycan acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from an UDP-glucose (UDP-Glc) donor onto a glycan acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto a glycan acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto a glycan acceptor. Mannosyltransferases comprise alpha-1,2-mannosyltransferases, alpha-1,3-mannosyltransferases and alpha-1,6-mannosyltransferases that transfer a Man residue from GDP-Man onto a glycan acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from an UDP-N-acetylglucosamine (UDP-GlcNAc) donor onto a glycan acceptor. N-acetylglucosaminyltransferases can be found but are not limited to GT2 and GT4 CAZy families. N-acetylgalactosaminyltransferases are glycosyltransferases that transfer an N-acetylgalactosamine group (GalNAc) from an UDP-N-acetylgalactosamine (UDP-GalNAc) donor onto a glycan acceptor. N-acetylgalactosaminyltransferases can be found but are not limited to GT7, GT12 and GT27 CAZy families. N-acetylmannosaminyltransferases are glycosyltransferases that transfer an N-acetylmannosamine group (ManNAc) from an UDP-N-acetylmannosamine (UDP-ManNAc) donor onto a glycan acceptor. Xylosyltransferases are glycosyltransferases that transfer a xylose residue (Xyl) from an UDP-xylose (UDP-Xyl) donor onto a glycan acceptor. Xylosyltransferases can be found but are not limited to GT14, GT61 and GT77 CAZy families. Glucuronyltransferases are glycosyltransferases that transfer a glucuronate from an UDP-glucuronate donor onto a glycan acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from an UDP-galacturonate donor onto a glycan acceptor. N-glycolylneuraminyltransferases are glycosyltransferases that transfer an N-glycolylneuraminic acid group (Neu5Gc) from a CMP-Neu5Gc donor onto a glycan acceptor. Rhamnosyltransferases are glycosyltransferases that transfer a rhamnose residue from a GDP-rhamnose donor onto a glycan acceptor. Rhamnosyltransferases can be found but are not limited to the GT1, GT2 and GT102 CAZy families. N-acetylrhamnosyltransferases are glycosyltransferases that transfer an N-acetylrhamnosamine residue from an UDP-N-acetyl-L-rhamnosamine donor onto a glycan acceptor. UDP-4-amino-4,6-dideoxy-N-acetyl-beta-L-altrosamine transaminases are glycosyltransferases that use an UDP-2-acetamido-2,6-dideoxy-L-arabino-4-hexulose in the biosynthesis of pseudaminic acid, which is a sialic acid-like sugar that is used to modify flagellin. Fucosaminyltransferases are glycosyltransferases that transfer an N-acetylfucosamine residue from a dTDP-N-acetylfucosamine or an UDP-N-acetylfucosamine donor onto a glycan acceptor.

The term “glycosylated product” as used herein refers to the group of molecules comprising at least one monosaccharide as defined herein. More in particular, the term glycosylated product is chosen from the list comprising, preferably consisting of, monosaccharide, phosphorylated monosaccharide, activated monosaccharide, disaccharide, oligosaccharide, glycoprotein and glycolipid.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-Iyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose, galactose, N-acetylglucosamine, glucosamine, mannose, xylose, N-acetylmannosamine, N-acetylneuraminic acid, N-glycolylneuraminic acid, N-acetylgalactosamine, galactosamine, fucose, rhamnose, glucuronic acid, gluconic acid, fructose and polyols.

The term “phosphorylated monosaccharide” as used herein refers to one of the above listed monosaccharides which is phosphorylated. Examples of phosphorylated monosaccharides include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The term “disaccharide” as used herein refers to a saccharide polymer containing two simple sugars, i.e. monosaccharides. Such disaccharides contain monosaccharides as described above and are preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc).

“Oligosaccharide” as the term is used herein and as generally understood in the state of the art, refers to a saccharide polymer containing a small number, typically three to twenty, of simple sugars, i.e. monosaccharides. Preferably the oligosaccharide as described herein contains monosaccharides selected from the list as used herein above. Examples of oligosaccharides include but are not limited to Lewis-type antigen oligosaccharides, mammalian (including human) milk oligosaccharides, 0-antigen, enterobacterial common antigen (ECA), the glycan chain present in lipopolysaccharides (LPS), capsular polysaccharides, peptidoglycan (PG), amino-sugars and antigens of the human ABO blood group system. As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

A ‘fucosylated oligosaccharide’ as used herein and as generally understood in the state of the art is an oligosaccharide that is carrying a fucose-residue. Examples comprise 2′-fucosyllactose (TEL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

As used herein, a ‘sialylated oligosaccharide’ is to be understood as a charged sialic acid containing oligosaccharide, i.e. an oligosaccharide having a sialic acid residue. It has an acidic nature. Some examples are 3-SL (3′-sialyllactose or 3′SL), 3′-sialyllactosamine, 6-SL (6′-sialyllactose or 6′SL), 6′-sialyllactosamine, oligosaccharides comprising 6′-sialyllactose, SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNac(3-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s), including but not limited to: oligosaccharide moieties of the gangliosides selected from GM3 (3′ sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAcβ-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAcβ-1,4(Neu5Acα-2,8Neu5Acα-2,8Neu5Acα2,3)Galβ-1,4Glc, GP1c Neu5Acα-2,8Neu5Acα-2,3Galβ-1,3GalNAcβ-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fuca-1,2Galβ-1,3GalNAcβ-1,4(Neu5Acα-2,3)Gal 13-1,4Glc; all of which may be extended to the production of the corresponding gangliosides by reacting the above oligosaccharide moieties with ceramide or synthetizing the above oligosaccharides on a ceramide.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (TEL), 3-fucosyllactose (3FL), 2′, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

Mammalian milk oligosaccharides comprise oligosaccharides present in milk found in any phase during lactation including colostrum milk from humans and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus).

As used herein the term “Lewis-type antigens” comprise the following oligosaccharides: H1 antigen, which is Fucα1-2Galβ1-3GlcNAc, or in short 2′FLNB; Lewisa, which is the trisaccharide Galβ1-3[Fucα1-4]GlcNAc, or in short 4-FLNB; Lewisb, which is the tetrasaccharide Fucα1-2Galβ1-3[Fucα1-4]GlcNAc, or in short DiF-LNB; sialyl Lewisa which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′ fucosyl-N-acetyl-lactosamine, in short 2′FLacNAc; Lewisx, which is the trisaccharide Galβ1-4[Fucα1-3]GlcNAc, or otherwise known as 3-Fucosyl-N-acetyl-lactosamine, in short 3-FLacNAc, Lewisy, which is the tetrasaccharide Fucα1-2Galβ1-4[Fucα1-3]GlcNAc and sialyl Lewisx which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria which are composed of a lipid A, a core oligosaccharide and the O-antigen. The term “capsular polysaccharides” refers to long-chain polysaccharides with repeat-unit structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. Said structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

As used herein, the term “glycoprotein” refers to any protein which contains a saccharide covalently attached to an amino acid side chain. Depending on the types of glycosylation, glycoproteins can be classified into N-, O-, P-, C-, or S-glycosylated or glypiated glycoproteins. In N-glycosylated glycoproteins the saccharide is attached to nitrogen, typically on the amide side chain of asparagine within the protein. In O-glycosylated glycoproteins the saccharide is attached to oxygen, typically on serine, threonine, tyrosine, hydroxylysine or hydroxyproline within the protein. In P-glycosylated glycoproteins the saccharide is attached to phosphorus on a phosphoserine within the protein. C-linked glycosylation refers to the covalent attachment of a mannose residue to a tryptophan residue within a protein. S-linked glycosylation refers to the attachment of a saccharide to the sulfur atom of cysteine within a protein. Glypiation refers to the process of adding a GPI (glycophosphatidylinositol) anchor to a protein. In this bond, mannose is linked to phosphoethanolamine, which in turn is attached to the terminal carboxyl group of the protein.

As used herein, the term “glycolipid” refers to any of the glycolipids which are generally known in the art. Glycolipids (GLs) can be subclassified into Simple (SGLs) and Complex (CGLs) glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like for example glycerol (glycoglycerolipids), peptide (glycopeptidolipids), acylated-sphingosine (glycosphingolipids), or other residues (lipopolysaccharides, phenolic glycolipids, nucleoside lipids).

The term “pathway for production of a glycosylated product” as used herein is a biochemical pathway consisting of the enzymes and their respective genes involved in the synthesis of a glycosylated product as defined herein. Said pathway for production of a glycosylated product comprises but is not limited to pathways involved in the synthesis of a nucleotide-activated sugar and the transfer of said nucleotide-activated sugar to an acceptor to create a glycosylated product of the present invention. Examples of such pathway for production of a glycosylated product comprise but is not limited to a fucosylation, sialylation, galactosylation, N-acetylglucosaminylation, N-acetylgalactosylation, mannosylation, N-acetylmannosinylation pathway.

A ‘fucosylation pathway’ as used herein is a biochemical pathway consisting of the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to α 1,2; α 1,3 α 1,4 or α 1,6 fucosylated oligosaccharides or fucosylated oligosaccharide containing glycosylated product.

A ‘sialylation pathway’ is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to α 2,3; α 2,6 α 2,8 sialylated oligosaccharides or sialylated oligosaccharide containing glycosylated product.

A ‘galactosylation pathway’ as used herein is a biochemical pathway consisting of the enzymes and their respective genes, galactose-1-epimerase, galactokinase, glucokinase, galactose-1-phosphate uridylyltransferase, UDP-glucose 4-epimerase, glucose-1-phosphate uridylyltransferase, and/or glucophosphomutase, combined with a galactosyltransferase leading to an alpha or beta bound galactose on the 2, 3, 4, 6 hydroxyl group of a mono, di, oligo or polysaccharide containing glycosylated product.

An ‘N-acetylglucosaminylation pathway’ as used herein is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, and/or glucosamine-1-phosphate acetyltransferase, combined with a glycosyltransferase leading to an alpha or beta bound N-acetylglucosamine on the 3, 4, 6 hydroxylgroup of a mono, di, oligo or polysaccharide containing glycosylated product.

An ‘N-acetylgalactosylation pathway’ as used herein is a biochemical pathway consisting of the enzymes and their respective genes, L-glutamine-D-fructose-6-phosphate aminotransferase, phosphoglucosamine mutase, N-acetylglucosamine 1-phosphate uridylyltransferase, UDP-N-acetylglucosamine 4-epimerase, UDP-galactose 4-epimerase, N-acetylgalactosamine kinase and/or UDP-GalNAc pyrophosphorylase combined with a glycosyltransferase leading to an alpha or beta bound N-acetylgalactosamine on a mono, di, oligo or polysaccharide containing glycosylated product.

A ‘mannosylation pathway’ as used herein is a biochemical pathway consisting of the enzymes and their respective genes, mannose-6-phosphate isomerase, phosphomannomutase and/or mannose-1-phosphate guanyltransferase combined with a glycosyltransferase leading to an alpha or beta bound mannose on a mono, di, oligo or polysaccharide containing glycosylated product.

An ‘N-acetylmannosinylation pathway’ as used herein is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to an alpha or beta bound N-acetylmannosamine on a mono, di, oligo or polysaccharide containing glycosylated product.

The term “non-native” as used herein with reference to a glycosylated product indicates that the glycosylated product is i) not naturally produced or ii) when naturally produced not in the same amounts by the cell; and that the cell has been genetically modified to be able to produce said glycosylated product or have a higher production of the glycosylated product.

The term “purified” refers to material that is substantially or essentially free from components which interfere with the activity of the glycosylated product. For cells, saccharides, nucleic acids, and polypeptides, the term “purified” refers to material that is substantially or essentially free from components when produced in a cultivation process like for example fermentation. Typically, purified saccharides, oligosaccharides, proteins, nucleic acids or glycosylated products as defined herein of the invention are at least about 50%, 55%, 60%, 65%, 70%, 75%, 80% or 85% pure, usually at least about %, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, purity can be determined using methods such as but not limited to thin layer chromatography, gas chromatography, NMR, HPLC, capillary electrophoresis or mass spectroscopy.

The term “cultivation” refers to the culture medium wherein the cell is cultivated or fermented, the cell itself, and the glycosylated product that is produced by the cell in whole broth, i.e. inside (intracellularly) as well as outside (extracellularly) of the cell.

The term “precursor” as used herein refers to substances which are taken up or synthetized by the cell for the specific production of a glycosylated product according to the present invention. In this sense a precursor can be an acceptor as defined herein, but can also be another substance, metabolite, which is first modified within the cell as part of the biochemical synthesis route of the glycosylated product. Examples of such precursors comprise the acceptors as defined herein, and/or glucose, galactose, fructose, glycerol, sialic acid, fucose, mannose, maltose, sucrose, lactose, glucose-1-phosphate, galactose-1-phosphate, UDP-glucose, UDP-galactose, glucose-6-phosphate, fructose-6-phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetyl-glucosamine-6-phosphate, N-acetyl-glucosamine, N-acetyl-mannosamine, N-acetylmannosamine-6-phosphate, UDP-N-acetylglucosamine, N-acetylglucosamine-1-phosphate, N-acetylneuraminic acid (sialic acid), N-acetyl-Neuraminic acid-9-phosphate, CMP-sialic acid, mannose-6-phosphate, mannose-1-phosphate, GDP-mannose, GDP-4-dehydro-6-deoxy-α-D-mannose, and/or GDP-fucose.

Optionally, the cell is transformed to comprise at least one nucleic acid sequence encoding a protein selected from the group consisting of lactose transporter, N-acetylneuraminic acid transporter, fucose transporter, transporter for a nucleotide-activated sugar wherein said transporter internalizes a to the medium added precursor for the synthesis of the glycosylated product.

The term “acceptor” as used herein refers to a mono-, di- or oligosaccharide which can be modified by a glycosyltransferase. Examples of such acceptors comprise glucose, galactose, fructose, glycerol, sialic acid, fucose, mannose, maltose, sucrose, lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of lacto-N-biose units, and oligosaccharide containing 1 or more N-acetyllactosamine units and/or 1 or more lacto-N-biose units or an intermediate into oligosaccharide, fucosylated and sialylated versions thereof.

### DETAILED DESCRIPTION OF THE INVENTION

According to a first aspect, the present invention provides a sucrose permease that has sucrose permease activity and comprises a polypeptide sequence that has at least 80% overall sequence identity to SEQ ID NO 01 and that (i) differs from SEQ ID NO 01 by having an amino acid change for proline at position 169 (P169a), for valine at position 316 (V316c) or for phenylalanine at position 371 (F371e) wherein a, c and e can be any amino acid residue excluding histidine for residue a, or (ii) shares the serine at position 246 of SEQ ID NO 01 and differs from SEQ ID NO 01 by having an amino acid change for tryptophan at position 230 (W230b) or for cysteine at position 327 (C327d) wherein b and d can be any amino acid residue, or (iii) differs from SEQ ID NO 01 by having at least two amino acid changes chosen from the list comprising proline at position 169 (P169f), tryptophan at position 230 (W230b), valine at position 316 (V316c), cysteine at position 327 (C327d) and phenylalanine at position 371 (F371e) wherein f, b, c, d and e can be any amino acid residue.

According to the present invention, the sucrose permease has sucrose permease activity and shares 80% overall sequence identity to SEQ ID NO 01. The polypeptide with SEQ ID NO 01 is the sucrose permease CscB from the E. coli W strain. The overall sequence identity of a polypeptide is determined by the program EMBOSS Needle 5.0 (https://galaxy-iuc.github.io/emboss-5.0-docs/needle.html), preferably with default parameters (the substitution matrix EBLOSUM62, the gap opening penalty 10, and the gap extension penalty 0.5) and preferably with sequences of mature proteins (i.e. without taking into account secretion signals or transit peptides).

Compared to overall sequence identity, the sequence identity will generally be higher when only conserved domains or motifs are considered. At least 80% overall sequence identity to the full length of the polypeptide with SEQ ID NO 01 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the polypeptide with SEQ ID NO 01 as given herein.

In an additional embodiment of the sucrose permease of the present invention, the sucrose permease comprises a single point mutation compared to SEQ ID NO 01 either at position P169 wherein P169 is changed into amino acid residue a, or at position V316 wherein V316 is changed into amino acid residue c, or at position F371 wherein F371 is changed into amino acid residue e, wherein a, c and e can be any amino acid residue excluding histidine for residue a.

In an alternative additional embodiment of the sucrose permease of the present invention, the sucrose permease shares the serine at position 246 (S246) of SEQ ID NO 01 and comprises a single point mutation compared to SEQ ID NO 01 either at position W230 wherein W230 is changed into amino acid residue b or at position C327 wherein C327 is changed into amino acid residue d, wherein b and d can be any amino acid residue.

In another alternative additional embodiment of the sucrose permease of the present invention, the sucrose permease comprises at least two single point mutations at positions chosen from the list comprising compared to SEQ ID NO 01 proline at position 169 (P169f), tryptophan at position 230 (W230b), valine at position 316 (V316c), cysteine at position 327 (C327d) and phenylalanine at position 371 (F371e) wherein f, b, c, d and e can be any amino acid residue. The sucrose permease of present invention thus includes a polypeptide sequence that has sucrose permease activity and that has at least % overall sequence identity to SEQ ID NO 01 with two, three, four or five specific single point mutations combined chosen from the list of P169f, W230b, V316c, C327d and F371e compared to SEQ ID NO 01, wherein f, b, c, d and e can be any amino acid residue.

In another embodiment of the sucrose permease of the present invention, the sucrose permease has sucrose permease activity and comprises a polypeptide sequence according to any one of SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32. The sucrose permease of present invention thus includes a polypeptide sequence wherein the CscB polypeptide of E. coli W is adapted with one single point mutation at either position P169, W230, V316, C327 or F371 as outlined in SEQ ID NOs 02, 03, 04, 05 and 06, respectively, or is adapted with two, three, four or five single point mutations chosen from the list containing P169, W230, V316, C327 or F371 and combined together as outlined in SEQ ID NOs 07, 08, 09, 10, 11, 12, 13, 14, 15, 16, 17, 18, 19, 20, 21, 22, 23, 24, 25, 26, 27, 28, 29, 30, 31 and 32, respectively, wherein indicated positions are mutated into any amino acid residue possible.

In an alternative embodiment of the sucrose permease of the present invention, the sucrose permease is a functional homologue, variant or derivative of any one of SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32, optionally having at least 80% overall sequence identity to the full length of any one of said polypeptides with SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32 and having sucrose permease activity. In another alternative embodiment of the sucrose permease of the present invention, the sucrose permease comprises a polypeptide comprising or consisting of an amino acid sequence having at least 80% overall sequence identity to the full length of any one of said polypeptides with SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32 and having sucrose permease activity.

At least 80% overall sequence identity to the full length of any one of the polypeptides with SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to any one of the polypeptides with SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32, respectively, as given herein.

In a preferred embodiment of the sucrose permease of the present invention, the sucrose permease is a polypeptide with SEQ ID NO 26 that differs from SEQ ID NO 01 by having an amino acid change for valine at position 316, an amino acid change for cysteine at position 327 and an amino acid change for phenylalanine at position 371 wherein said valine, cysteine and phenylalanine have been changed into any amino acid residue possible. In a more preferred embodiment of the sucrose permease of the present invention, the sucrose permease is a polypeptide with SEQ ID NO 26 that differs from SEQ ID NO 01 by having an amino acid change for valine at position 316 into alanine, an amino acid change for cysteine at position 327 into glycine and an amino acid change for phenylalanine at position 371 into valine as is outlined in SEQ ID NO 33. In an alternative preferred embodiment of the sucrose permease of the present invention, the sucrose permease is a functional homologue, variant or derivative of SEQ ID NO 33 that optionally has at least 80% overall sequence identity to the full length of said polypeptide with SEQ ID NO 33 and that has sucrose permease activity. In another preferred embodiment of the sucrose permease of the present invention, the sucrose permease comprises a polypeptide comprising or consisting of an amino acid sequence having at least 80% overall sequence identity to the full length of said polypeptide with SEQ ID NO 33 and that has sucrose permease activity.

At least 80% overall sequence identity to the full length of the polypeptide with SEQ ID NO 33 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the polypeptide with SEQ ID NO 33 as given herein.

According to another embodiment of the present invention, the sucrose permease further differs from SEQ ID NO 01 by having any one or more amino acid changes chosen from the list comprising leucine at position 61 into proline, tryptophan, histidine, phenylalanine, or tyrosine; phenylalanine at position 159 into leucine; glycine at position 162 into cysteine; proline at position 169 into histidine; arginine at position 300 into alanine or leucine; glutamine at position 353 into histidine; truncation of amino acid residues 404 to 415 and truncation of amino acid residues 409 to 415. Said sucrose permease thus comprises a polypeptide sequences that has at least 80% overall sequence identity to SEQ ID NO 01 and that (i) differ from SEQ ID NO 01 by having an amino acid change for proline at position 169 (P169a), for valine at position 316 (V316c) or for phenylalanine at position 371 (F371e) wherein a, c and e can be any amino acid residue excluding histidine for residue a, or (ii) share the serine at position 246 of SEQ ID NO 01 and differs from SEQ ID NO 01 by having an amino acid change for tryptophan at position 230 (W230b) or for cysteine at position 327 (C327d) wherein b and d can be any amino acid residue, or (iii) differ from SEQ ID NO 01 by having at least two amino acid changes chosen from the list comprising proline at position 169 (P169f), tryptophan at position 230 (W230b), valine at position 316 (V316c), cysteine at position 327 (C327d) and phenylalanine at position 371 (F371e) wherein f, b, c, d and e can be any amino acid residue, and that have at least one additional change compared to SEQ ID NO 01 chosen from the list comprising L61P, L61W, L61H, L61F, L61Y, F159L, G162C, P169H, R300A, R300L, Q353H and a truncation of 7 C-terminal amino acid residues (truncation of residues 409 to 415) or 12 C-terminal amino acid residues (truncation of residues 404 to 415).

According to a preferred embodiment of the sucrose permease of the present invention, the sucrose permease has improved affinity for sucrose compared to the sucrose permease of E. coli W with SEQ ID NO 01. In the context of the present invention, an improved affinity for sucrose preferably means that a sucrose permease according to the invention has a lower Km for sucrose than the sucrose permease of E. coli W with SEQ ID NO 01, preferably wherein said Km of each sucrose permease is determined by the same method and under identical conditions. The Km for sucrose can be assessed by a person skilled in the art using routinely available methods such as described by for example Sugihara et al. (2011) Biochemistry 50(51): 11009-11014.

According to another preferred embodiment of the sucrose permease of the present invention, said sucrose permease comprises or consists of at least 200, preferably at least 225, more preferably at least 250 amino acids; and/or consists of less than 450, preferably less than 440, more preferably of less than 430 amino acids. In this context, said sucrose permease preferably (i) comprises a polypeptide sequence according to any one of SEQ ID Nos 58 to 62; (ii) is a functional homologue, variant or derivative of any one of SEQ ID Nos 58 to 62 and optionally having at least 80% overall sequence identity to the full length of any of said polypeptides with SEQ ID NO 58, 59, 60, 61 or 62; and/or (iii) comprises a polypeptide comprising or consisting of an amino acid sequence having at least 80% overall sequence identity to the full length of any one of said polypeptides with SEQ ID NO 58, 59, 60, 61 or 62.

According to a more preferred embodiment of the sucrose permease of the present invention, said sucrose permease comprises or consists of at least 350, preferably at least 360, more preferably at least 370, even more preferably at least 380, even more preferably at least 390, most preferably at least 400 amino acids; and/or consists of less than 450, preferably less than 440, more preferably less than 430 amino acids. In this context, said sucrose permease preferably (i) comprises a polypeptide sequence with SEQ ID NO 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56 or 57; (ii) is a functional homologue, variant or derivative of any one of SEQ ID Nos 34, 03, 04, 05, 06, 07, 08, 09, 10, 11, 12, 13, 14, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56 or 57 and optionally having at least 80% overall sequence identity to the full length of any of said polypeptides with SEQ ID Nos 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56 or 57; and/or (iii) comprises a polypeptide comprising or consisting of an amino acid sequence having at least 80% overall sequence identity to the full length of any one of said polypeptides with SEQ ID NO 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56 or 57.

According to a second aspect, the present invention provides a metabolically engineered cell for the production of a glycosylated product. Herein, a metabolically engineered cell comprising a pathway for the production of a glycosylated product is provided which expresses at least one sucrose permease which has sucrose permease activity and comprises a polypeptide sequence that differs from SEQ ID NO 01 by having any one or more of the amino acid changes chosen from the list comprising: proline at position 169 (P169f), tryptophan at position 230 (W230b), valine at position 316 (V316c), cysteine at position 327 (C327d) or phenylalanine at position 371 (F371e) wherein f, b, c, d and e can be any amino acid residue. Alternatively, a metabolically engineered cell comprising a pathway for the production of a glycosylated product is provided which expresses at least one sucrose permease according to the first aspect of present invention.

According to a third aspect, the present invention provides a method for the production of a glycosylated product. The method comprises the steps of:
i) providing a metabolically engineered cell comprising a pathway for the production of said glycosylated product, said cell expresses at least one sucrose permease which has sucrose permease activity and comprises a polypeptide sequence that differs from SEQ ID NO 01 by having any one or more of the amino acid changes chosen from the list comprising: proline at position 169 (P169f), tryptophan at position 230 (W230b), valine at position 316 (V316c), cysteine at position 327 (C327d) or phenylalanine at position 371 (F371e) wherein f, b, c, d and e can be any amino acid residue, and
ii) cultivating said cell under conditions permissive to produce said glycosylated product,
iii) preferably, separating said glycosylated product from said cultivation.

Alternatively, the present invention provides a method for the production of a glycosylated product, the method comprises the steps of;
i) providing a metabolically engineered cell according to the second aspect of the present invention, and
ii) cultivating said cell under conditions permissive to produce said glycosylated product,
iii) preferably, separating said glycosylated product from said cultivation.

In the scope of the present invention, permissive conditions are understood to be conditions relating to physical or chemical parameters including but not limited to temperature, pH, pressure, osmotic pressure and product/educt concentration.

In a particular embodiment, the permissive conditions may include a temperature-range of 30+/−20 degrees centigrade, a pH-range of 7+/−3.

The present invention provides different types of cells for the production of a glycosylated product with a metabolically engineered cell.

According to one embodiment of the method and/or cell of the present invention, the cell preferably expresses at least one sucrose permease that has sucrose permease activity and that comprises at least one single point mutation compared to SEQ ID NO 01, chosen from the list comprising: proline at position 169 (P169f), tryptophan at position 230 (W230b), valine at position 316 (V316c), cysteine at position 327 (C327d) or phenylalanine at position 371 (F371e) wherein f, b, c, d and e can be any amino acid residue. The cell of present invention thus expresses at least one sucrose permease with sucrose permease activity that is a polypeptide sequence that has, compared to SEQ ID NO 01, one, two, three, four or five specific single point mutations chosen from the list of P169f, W230b, V316c, C327d and F371e, wherein f, b, c, d and e can be any amino acid residue.

In another embodiment of the method and/or cell of the present invention, at least one of the sucrose permeases expressed in the cell has at least 80% overall sequence identity to SEQ ID NO 01. At least 80% overall sequence identity to the full length of the polypeptide with SEQ ID NO 01 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the polypeptide with SEQ ID NO 01 as given herein.

In a preferred embodiment of the method and/or cell of the present invention, the cell expresses at least one sucrose permease that comprises a polypeptide sequence according to any one of SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62. In an alternative preferred embodiment of the method and/or cell of the present invention, the cell expresses at least one sucrose permease that is a functional homologue, variant or derivative of any one of SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62, optionally having at least 80% overall sequence identity to the full length of any one of said polypeptides with SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62, having at least one amino acid change chosen from the list comprising: proline at position 169 (P169f), tryptophan at position 230 (W230b), valine at position 316 (V316c), cysteine at position 327 (C327d) or phenylalanine at position 371 (F371e) wherein f, b, c, d and e can be any amino acid residue and having sucrose permease activity. At least 80% overall sequence identity to the full length of any one of said polypeptides with SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to any one of the polypeptides with SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62, respectively, as given herein. In another alternative preferred embodiment of the method and/or cell of the present invention, the cell expresses at least one sucrose permease that comprises a polypeptide comprising or consisting of an amino acid sequence having at least 80% overall sequence identity to the full length of any one of said polypeptides with SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62, having at least one amino acid change chosen from the list comprising: proline at position 169 (P169f), tryptophan at position 230 (W230b), valine at position 316 (V316c), cysteine at position 327 (C327d) or phenylalanine at position 371 (F371e) wherein f, b, c, d and e can be any amino acid residue and having sucrose permease activity.

In another preferred embodiment of the method and/or cell of the present invention, the cell expresses a sucrose permease with SEQ ID NO 26 wherein said sucrose permease with SEQ ID NO 26 differs from SEQ ID NO 01 by having an amino acid change for valine at position 316 to alanine, for cysteine at position 327 to glycine and for phenylalanine at position 371 to valine as outlined in SEQ ID NO 33. In an alternative preferred embodiment of the method and/or cell of the present invention, the cell expresses a functional homologue, variant or derivative of SEQ ID NO 33 optionally having at least 80% overall sequence identity to the full length of said polypeptide with SEQ ID NO 33 and having at least 80% overall sequence identity to the full length of said polypeptide with SEQ ID NO 01 and having sucrose permease activity. In another preferred embodiment of the method and/or cell of the present invention, the cell expresses a sucrose permease comprising a polypeptide comprising or consisting of an amino acid sequence having at least % overall sequence identity to the full length of said polypeptide with SEQ ID NO 33 and that has sucrose permease activity. At least 80% overall sequence identity to the full length of the polypeptide with SEQ ID NO 33 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the polypeptide with SEQ ID NO 33 as given herein.

According to another embodiment of the method and/or cell of the present invention, the cell expresses a sucrose permease that has sucrose permease activity and that has, compared to SEQ ID NO 01, one, two, three, four or five specific single point mutations chosen from the list of P169f, W230b, V316c, C327d and F371e, wherein f, b, c, d and e can be any amino acid residue, and that further differs from SEQ ID NO 01 by having any one or more amino acid changes chosen from the list comprising leucine at position 61 into proline (L61P), tryptophan (L61W), histidine (L61H), phenylalanine (L61F), or tyrosine (L61Y); phenylalanine at position 159 into leucine (F159L); glycine at position 162 into cysteine (G162C); proline at position 169 into histidine (P169H); arginine at position 300 into alanine (R300A) or leucine (R300L); glutamine at position 353 into histidine (Q353H); a truncation of amino acid residues 404 to 415 and a truncation of amino acid residues 409 to 415. According to another preferred embodiment of the method and/or cell of the present invention, said further mutated sucrose permeases have at least 80% overall sequence identity to SEQ ID NO 01.

The present invention provides different types of metabolically engineered cells that express different types of sucrose permeases for the production of a glycosylated product. For example, the present invention provides a cell comprising a pathway for the production of glycosylated product that expresses a sucrose permease that has sucrose permease activity and differs from the cscB sucrose permease of E. coli W with SEQ ID NO 01 by one single point mutation chosen from P169f, W230b, V316c, C327d or F371e, wherein f, b, c, d and e can be any amino acid residue, respectively, e.g. a sucrose permease like any one of the polypeptides with SEQ ID NOs 35, 36, 37, 98, 99, 100, 101, 102, 103, 104, 105, 106, 107, 108, 109, 119, 120 or 121, wherein said polypeptides have sucrose permease activity and have a single point mutation of V316A as for SEQ ID NO 35; a single point mutation of V316C as for SEQ ID NO 98; a single point mutation of V316F as for SEQ ID NO 99; a single point mutation of C327A as for SEQ ID NO 100; a single point mutation of C327G as for SEQ ID NO 36; a single point mutation of F371A as for SEQ ID NO 105; a single point mutation of F371C as for SEQ ID NO 107; a single point mutation of F371E as for SEQ ID NO 102; a single point mutation of F371G as for SEQ ID NO 119; a single point mutation of F3711 as for SEQ ID NO 109; a single point mutation of F371L as for SEQ ID NO 104; a single point mutation of F371M as for SEQ ID NO 120; a single point mutation of F371N as for SEQ ID NO 121; a single point mutation of F371Q as for SEQ ID NO 103; a single point mutation of F371S as for SEQ ID NO 108; a single point mutation of F371T as for SEQ ID NO 106; a single point mutation of F371V as for SEQ ID NO 37; a single point mutation of F371Y as for SEQ ID NO 101. The present invention also provides a cell comprising a pathway for the production of glycosylated product that expresses a sucrose permease that has sucrose permease activity, and differs from the cscB sucrose permease of E. coli W with SEQ ID NO 01 by more than one single point mutation wherein one of said single point mutations is chosen from P169f, W230b, V316c, C327d or F371e, wherein f, b, c, d and e can be any amino acid residue, respectively, e.g. a sucrose permease like any one of the polypeptides with SEQ ID NOs 41, 42, 43, 44, 45, 46, 47, 48, 49, 50, 51, 52, 53, 54, 55, 56, 57, 58, 59, 60, 61 or 62 wherein said polypeptides have sucrose permease activity and have amongst other point mutations but not at positions 169, 230, 327 or 371 compared to SEQ ID NO 01 a single point mutation of V316A as for SEQ ID NOs 41, 42, 43, 44, 45, 46, 47 or 48; amongst other point mutations but not at positions 169, 230, 316 or 371 compared to SEQ ID NO 01 a single point mutation of C327G as for SEQ ID NOs 49, 50, 51, 52, 53, 54, 55, 56 or 57; amongst other point mutations but not at positions 169, 316, 327 or 371 compared to SEQ ID NO 01 a single point mutation of W230L as for SEQ ID NOs 58, 59, 60, 61 or 62. The present invention also provides a cell comprising a pathway for the production of a glycosylated product wherein said cell expresses two or more sucrose permeases that have sucrose permease activity and each differ from SEQ ID NO 01 by one single point mutation chosen from P169f, W230b, V316c, C327d or F371e, wherein f, b, c, d and e can be any amino acid residue, respectively. In this way, the present invention also provides a cell that expresses two or more sucrose permeases chosen from the list comprising a polypeptide with SEQ ID 35, 36, 37, 41, 42, 43, 44, 45, 46, 47, 48, 49, 50, 51, 52, 53, 54, 55, 56, 57, 58, 59, 60, 61, 62, 98, 99, 100, 101, 102, 103, 104, 105, 106, 107, 108, 109, 119, 120 and 121. The present invention also provides a cell that expresses the cscB sucrose permease of E. coli W with SEQ ID NO 01 and a sucrose permease chosen from the list comprising SEQ ID NOs 35, 36, 37, 41, 42, 43, 44, 45, 46, 47, 48, 49, 50, 51, 52, 53, 54, 55, 56, 57, 58, 59, 60, 61, 62, 98, 99, 100, 101, 102, 103, 104, 105, 106, 107, 108, 109, 119, 120 and 121. The present invention also provides a cell that expresses SEQ ID NO 01 and two or more polypeptides chosen from the list comprising SEQ ID NOs 35, 36, 37, 41, 42, 43, 44, 45, 46, 47, 48, 49, 50, 51, 52, 53, 54, 55, 56, 57, 58, 59, 60, 61, 62, 98, 99, 100, 101, 102, 103, 104, 105, 106, 107, 108, 109, 119, 120 and 121.

The present invention also provides a cell comprising a pathway for the production of a glycosylated product that expresses a sucrose permease that has sucrose permease activity and differs from SEQ ID NO 01 by two single point mutations chosen from P169f, W230b, V316c, C327d or F371e, wherein f, b, c, d and e can be any amino acid residue, respectively, e.g. a sucrose permease like any one of the polypeptides with SEQ ID NOs 38, 39 or 40, wherein said polypeptides have sucrose permease activity and have a single point mutation of V316A and of C327G as for SEQ ID NO 38; a single point mutation of V316A and of F371V as for SEQ ID NO 39; a single point mutation of C327G and of F371V as for SEQ ID NO 40.

The present also provides a cell comprising a pathway for the production of a glycosylated product wherein said cell expresses two or more sucrose permeases that have sucrose permease activity and each differ from SEQ ID NO 01 by two single point mutations chosen from P169f, W230b, V316c, C327d or F371e, wherein f, b, c, d and e can be any amino acid residue, respectively. In this way, the present invention also provides a cell wherein said cell expresses two or three sucrose permeases chosen from the list comprising a polypeptide with SEQ ID 38, 39 and 40. The present invention also provides a cell that expresses the cscB sucrose permease of E. coli W with SEQ ID NO 01 and a sucrose permease chosen from the list comprising SEQ ID NOs 38, 39 and 40. The present invention also provides a cell that expresses SEQ ID NO 01 and two or three polypeptides chosen from the list comprising SEQ ID NOs 38, 39 and 40. The present invention also provides a cell comprising a pathway for the production of a glycosylated product that expresses a sucrose permease that has sucrose permease activity and differs from SEQ ID NO 01 by three or more single point mutations chosen from P169f, W230b, V316c, C327d or F371e, wherein f, b, c, d and e can be any amino acid residue, respectively, e.g. a sucrose permease like any one of the polypeptides with SEQ ID NOs 33, 110, 111, 112, 113 or 114, wherein said polypeptides have sucrose permease activity and have single point mutations of V316A, C327G and F371V as for SEQ ID NO 33; single point mutations of V3161, C327A and F371T as for SEQ ID NO 110, single point mutations of V316M, C327G and F371G as for SEQ ID NO 111; single point mutations of V316L, C327G and F371S as for SEQ ID NO 112; single point mutations of V316F, C327G, F371V as for SEQ ID NO 113; single point mutation of V3161, C327G and F371C as for SEQ ID NO 114. The present also provides a cell comprising a pathway for the production of a glycosylated product wherein said cell expresses two or more sucrose permeases that have sucrose permease activity and each differ from SEQ ID NO 01 by three or more single point mutations chosen from P169f, W230b, V316c, C327d or F371e, wherein f, b, c, d and e can be any amino acid residue, respectively. In this way, the present also provides a cell wherein said cell expresses two or more sucrose permeases chosen from the list comprising a polypeptide with SEQ ID NOs 33, 110, 111, 112, 113 or 114. The present invention also provides a cell comprising a pathway for the production of a glycosylated product that expresses the cscB sucrose permease of E. coli W with SEQ ID NO 01 and a sucrose permease chosen from the list comprising SEQ ID NOs 33, 110, 111, 112, 113 or 114. The present invention also provides a cell that expresses SEQ ID NO 01 and two or more polypeptides chosen from the list comprising SEQ ID NOs 33, 110, 111, 112, 113 or 114.

The present invention also provides a cell comprising a pathway for the production of a glycosylated product that expresses a sucrose permease that has sucrose permease activity and differs from SEQ ID NO 01 by at least one single point mutation chosen from P169f, W230b, V316c, C327d or F371e, wherein f, b, c, d and e can be any amino acid residue, respectively, and that further differs from SEQ ID NO 01 by having any one or more of the single point mutation chosen from L61P, L61W, L61H, L61F, L61Y, F159L, G162C, P169H, R300A, R300L, Q353H and/or a truncation of amino acid residues 404 to 415 or amino acid residues 409 to 415, e.g. a sucrose permease like SEQ ID NO 117 or 118, wherein said polypeptides have sucrose permease activity and differ from SEQ ID NO 01 by C327G and F371V mutations, and that further differ from SEQ ID NO 01 by a C-terminal truncation of 12 or 7 amino acid residues, as is for SEQ ID NO 117 or 118, respectively.

The present invention also provides a cell comprising a pathway for the production of a glycosylated product that expresses two or more sucrose permeases that differ from SEQ ID NO 01 and from each other by the number of mutations chosen from P169f, W230b, V316c, C327d or F371e, wherein f, b, c, d and e can be any amino acid residue, respectively. In this way, the present invention also provides a cell that expresses two or more sucrose permeases wherein, e.g. the first sucrose permease differs from SEQ ID NO 01 by one amino acid change chosen from P169f, W230b, V316c, C327d or F371e and the second sucrose permease differs from SEQ ID NO 01 by two or more amino acid changes chosen from P169f, W230b, V316c, C327d or F371e. The present invention also provides a cell comprising a pathway for the production of a glycosylated product that expresses two or more sucrose permeases chosen from the list comprising SEQ ID NOs 33, 35, 36, 37, 38, 39, 40, 41, 42, 43, 44, 45, 46, 47, 48, 49, 50, 51, 52, 53, 54, 55, 56, 57, 58, 59, 60, 61, 62, 98, 99, 100, 101, 102, 103, 104, 105, 106, 107, 108, 109, 110, 111, 112, 113, 114, 117,118, 119, 120 and 121.

The present invention additionally provides different methods with said different cells for the production of a glycosylated product.

According to the present invention, a change of an amino acid at a particular position in a polypeptide sequence (e.g. SEQ ID NO 01) into any amino acid residue should be understood as a substitution of an amino acid at a given position in a polypeptide sequence (e.g. SEQ ID NO 01) into an amino acid that is different from the original amino acid and that is chosen from the list comprising both classical and non-classical amino acid residues and chemical amino acid analogues. For example, a sucrose permease according to the invention that differs from SEQ ID NO 01 by having an amino acid change for phenylalanine at position 371 (F371e, wherein e can by any amino acid residue) means in the context of the invention that the sucrose permease according to the invention has an amino acid residue at position 371 which is different from phenylalanine. Preferably, the ‘Xaa’ amino acid residue(s) as present in any one of SEQ ID NOs 02 to 032 and 034 of the sequence listing can be any amino acid (classical, non-classical, chemical amino acid analogues), unless specifically stated otherwise, except for the amino acid which is present at this position in SEQ ID NO 01. For example, the sucrose permease according to SEQ ID NO 26 has an amino acid at position 316 which is different from valine, an amino acid at position 327 which is different from cysteine and an amino acid at position 371 which is different from phenylalanine. Classical amino acid residues are known by the person skilled in the art as the standard or canonical amino acid residues that are encoded by the universal genetic code and refer to any one of the amino acid residues of the list comprising alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), valine (Val, V). Non-classical amino acid residues and chemical amino acid analogues refer to any one of the molecules of the list comprising but not limited to selenocysteine (Sec, U), phenylselenocysteine, pyrrolysine (Pyl, O), hydroxyproline, carboxyglutamate, hypusine, 2-methylalanine, beta-alanine, ornithine, citrulline, N-formylmethionine, S-adenosylmethionine, mimosine, 5-hydroxytryptophan, L-dihydroxyphenylalanine, o-methyltyrosine, L-3-(2-naphthyl)alanine, I-(7-hydroxycoumarin-4-yl) ethylglycine, p-acetylphenylalanine, m-acetylphenylalanine, p-(3-oxobutanoyl)-L-phenylalanine, p-(2-amino-3-hydroxyethyl)phenylalanine, p-isopropylthiocarbonyl-phenylalanine, p-ethylthiocarbonylphenylalanine, p-propargyloxyphenylalanine, p-azidophenylalanine, p-benzoyl-L-phenylalanine, O-(2-nitrobenzyl)tyrosine, S-(2-nitrobenzyl)cysteine, p-azophenyl-phenylalanine, p-methoxyphenylalanine, fluorescent amino acids with 7-hydroxycoumarin and dansyl side chains, p-iodo-L-phenylalanine, beta-GlcNAc-serine, alpha-GalNAc-threonine, p-Carboxymethylphenylalanine, 3,4-dihydroxy-L-phenylalanine, 3-amino-L-tyrosine, biphenylalanine, p-amino-L-phenylalanine, p-isopropyl-L-phenylalanine, alpha-aminocaprylic acid, L-homoglutamine, p-nitro-L-phenylalanine, bipyridyl-containing amino acid, 3-iodotyrosine, homoallyglycine, homoproparglycine, norvaline, norleucine, cis-crotylglycine, trans-crotylglycine, 2-aminoheptanoic acid, 2-butynylglycine, allylglycine, o-allylserine, propargylglycine, Selenomethionine (Sem), telluromethionine, trifluoromethionine, ethionine, 6,6,6-trifluoro-2-amino hexanoic acid, methoxinine (Mox), telluromethionine (Tem), tellurocysteine (Tec), beta-[2,3-b]thienopyrrolylalanine, beta-[3,2-b]thienopyrrolylalanine, beta-[2,3-b]selenopyrrolylalanine, beta-[3,2-b]selenolopyrrolylalanine, beta-2-thienylalanine, beta-2-selenielylalanine, beta-2-tellurienylalanine, beta-3-selenienylalanine.

According to the present invention, the amino acid residue a can be any amino acid residue excluding histidine. The term “any amino acid residue excluding histidine” should be understood as any classical amino acid residue, non-classical amino acid residue or chemical amino acid analogue as enlisted herein but not histidine. Within the present invention, amino acid residue a is chosen from the list comprising alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), valine (Val, V) and non-classical amino acid residues and chemical amino acid analogues as described herein.

According to a preferred embodiment of the present invention, the amino acid residue a is threonine, the amino acid residue b is leucine, the amino acid residue c is chosen from the list comprising alanine, cysteine and phenylalanine, the amino acid residue d is chosen from the list comprising alanine and glycine, the amino acid residue e is chosen from the list comprising alanine, cysteine, glutamate, glycine, histidine, isoleucine, lysine, methionine, asparagine, glutamine, serine, threonine, valine and tyrosine, and the amino acid residue f is threonine.

According to a more preferred embodiment of the present invention, the amino acid residue c is alanine, the amino acid residue d is glycine, and the amino acid e is valine.

In a preferred embodiment of the cell of present invention, the cell is metabolically engineered to comprise a pathway for the production of a glycosylated product as defined herein. In an alternative preferred embodiment of the cell of present invention, the cell is metabolically engineered for modified expression or activity of a sucrose permease of present invention. In an additional alternative preferred embodiment of the cell of present invention, the cell is metabolically engineered to comprise a pathway for the production of a glycosylated product and to have modified expression or activity of a sucrose permease of present invention.

According to a preferred embodiment of the method and cell according to the invention, the metabolically engineered cell is modified with gene expression modules wherein the expression from any one of said expression modules is constitutive or is tuneable.

Said expression modules are also known as transcriptional units and comprise polynucleotides for expression of recombinant genes including coding gene sequences and appropriate transcriptional and/or translational control signals that are operably linked to the coding genes. Said control signals comprise promoter sequences, untranslated regions, ribosome binding sites, terminator sequences. Said expression modules can contain elements for expression of one single recombinant gene but can also contain elements for expression of more recombinant genes or can be organized in an operon structure for integrated expression of two or more recombinant genes. Said polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred embodiment of the present invention, the cell is modified with one or more expression modules. The expression modules can be integrated in the genome of said cell or can be presented to said cell on a vector. Said vector can be present in the form of a plasmid, cosmid, phage, liposome, or virus, which is to be stably transformed/transfected into said metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the invention. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. Said recombinant gene is involved in the expression of a polypeptide acting in the synthesis of said glycosylated product; or said recombinant gene is linked to other pathways in said host cell that are not involved in the synthesis of said glycosylated product. Said recombinant genes encode endogenous proteins with a modified expression or activity, preferably said endogenous proteins are overexpressed; or said recombinant genes encode heterologous proteins that are heterogeneously introduced and expressed in said modified cell, preferably overexpressed. The endogenous proteins can have a modified expression in the cell which also expresses a heterologous protein.

According to a preferred embodiment of the present invention, the expression of each of said expression modules is constitutive or tuneable as defined herein.

In a further embodiment of the method and/or cell of the invention, the cell is modified in the expression or activity of at least one of said sucrose permeases. In a preferred embodiment, said sucrose permease is an endogenous protein of the cell with a modified expression or activity, preferably said endogenous sucrose permease is overexpressed; alternatively said sucrose permease is an heterologous protein that is heterogeneously introduced and expressed in said cell, preferably overexpressed. Said endogenous sucrose permease can have a modified expression in the cell which also expresses a heterologous sucrose permease.

According to one embodiment of the method and/or cell of the invention, the cell is capable to synthesize a nucleotide-activated sugar to be used in the production of said glycosylated product. In a preferred embodiment of the method and/or cell of the invention, the cell is capable to synthesize one or more nucleotide-activated sugars chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.

GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. Said modification can be any one or more chosen from the group comprising knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

CMP-Neu5Ac can be provided by an enzyme expressed in the cell or by the metabolism of the cell. Such cell producing CM P-Neu5Ac can express an enzyme converting, e.g., sialic acid, which is to be added to the cell, to CM P-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. Said modification can be any one or more chosen from the group comprising knock-out of an N-acetylglucosamine-6-phosphate deacetylase, knock-out of an glucosamine-6-phosphate deaminase, over-expression of a sialate synthase encoding gene, and over-expression of an N-acetyl-D-glucosamine-2-epimerase encoding gene.

UDP-GlcNAc can be provided by an enzyme expressed in the cell or by the metabolism of the cell. Such cell producing an UDP-GlcNAc can express enzymes converting, e.g. GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a a phosphoglucosamine mutase, and over-expression of an N-acetylglucosamine-1-phosphate uridyltransferase/glucosamine-1-phosphate acetyltransferase.

UDP-Gal can be provided by an enzyme expressed in the cell or by the metabolism of the cell. Such cell producing UDP-Gal can express an enzyme converting, e.g. UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. Said modification can be any one or more chosen from the group comprising knock-out of an bifunctional 5′-nucleotidase/UDP-sugar hydrolase encoding gene, knock-out of a galactose-1-phosphate uridylyltransferase encoding gene and over-expression of an UDP-glucose-4-epimerase encoding gene.

UDP-GalNAc can be synthesized from UDP-GlcNAc by the action of a single-step reaction using an UDP-N-acetylglucosamine 4-epimerase like e.g. wbgU from Plesiomonas shigelloides, gne from Yersinia enterocolitica or wbpP from Pseudomonas aeruginosa serotype 06. Preferably, the cell is modified to produce UDP-GalNAc. More preferably, the cell is modified for enhanced UDP-GalNAc production.

UDP-ManNAc can be synthesized directly from UDP-GlcNAc via an epimerization reaction performed by an UDP-GlcNAc 2-epimerase (like e.g. cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

CMP-Neu5Gc can be synthesized directly from CMP-Neu5Ac via a hydroxylation reaction performed by a vertebrate CMP-Neu5Ac hydroxylase (CMAH) enzyme. Preferably, the cell is modified to produce CMP-Neu5Gc. More preferably, the cell is modified for enhanced CMP-Neu5Gc production.

According to another aspect of the method and/or cell of the invention, the cell is capable to express at least one glycosyltransferase chosen from the list comprising fucosyltransferases, sialyltransferases, galactosyltransferases, glucosyltransferases, mannosyltransferases, N-acetylglucosaminyltransferases, N-acetylgalactosam inyltransferases, N-acetyl mannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases and fucosaminyltransferases. In a further aspect of the method and/or cell of the invention, the cell is modified in the expression or activity of at least one of said glycosyltransferases. In a preferred embodiment, said glycosyltransferase is an endogenous protein of the cell with a modified expression or activity, preferably said endogenous glycosyltransferase is overexpressed; alternatively said glycosyltransferase is a heterologous protein that is heterogeneously introduced and expressed in said cell, preferably overexpressed. Said endogenous glycosyltransferase can have a modified expression in the cell which also expresses a heterologous glycosyltransferase.

According to another embodiment of the method and/or cell of the invention, the cell is engineered with at least one gene encoding for an enzyme enabling the cell to split sucrose into an activated saccharide and a monosaccharide. In a further embodiment of the method and/or cell of the invention, said enzyme that splits sucrose into an activated saccharide and a monosaccharide is chosen from the list comprising sucrose phosphorylases, sucrose synthases, sucrases (invertases) combined with a glucokinase and/or a fructokinase, a trehalase combined with a glucokinase, a maltase combined with a glucokinase, a sucrose-6-phosphate hydrolase combined with a fructokinase, a maltose phosphorylase, a maltose synthase, an amylase combined with a phosphorylase or synthase or hydrolase, a lactose synthase, a lactose phosphorylase, a lactase (or beta-galactosidase) combined with a galactokinase and/or a glucokinase.

According to an additional embodiment of the method and/or cell of the invention, the cell is engineered so that at least one gene encoding for an enzyme which converts said activated saccharide into biomass and/or bio-catalytic enzymes is rendered less-functional or non-functional. In a further aspect of the method and/or cell of the invention, said gene which encodes for an enzyme which converts said activated saccharide into biomass and/or bio-catalytic enzymes is chosen from the list comprising genes encoding beta-galactosidase, phosphoglucomutase, glucose-1-phosphate adenylyltransferase, phosphatase, glucose-1-phosphate uridyltransferase, UDP-glucose-4-epimerase, UDP-glucose:galactose-1-phosphate uridyltransferase, UDP-galactopyranose mutase, UDP-galactose:(glucosyl)lipopolysaccharide-1,6-galactosyltransferase, UDP-galactosyltransferase, UDP-glucosyltransferase, UDP-glucuronate transferase, UDP-glucose lipid carrier transferase, UDP-sugar hydrolase, invertase, maltase, trehalase, sugar transporting phosphotransferase and hexokinase.

In a further preferred embodiment, the cell described herein is using a split metabolism having a production pathway and a biomass pathway as described in WO2012/007481, which is herein incorporated by reference. Said organism can, for example, be genetically modified to accumulate fructose-6-phosphate by altering the genes selected from the phosphoglucoisomerase gene, phosphofructokinase gene, fructose-6-phosphate aldolase gene, fructose isomerase gene, and/or fructose:PEP phosphotransferase gene.

According to the present invention, the method as described herein preferably comprises a step of separating said glycosylated product from said cultivation.

The terms “separating from said cultivation” means harvesting, collecting, or retrieving said glycosylated product from the cell and/or the medium of its growth.

The glycosylated product can be separated in a conventional manner from the aqueous culture medium, in which the cell was grown. In case said glycosylated product is still present in the cells producing the glycosylated product, conventional manners to free or to extract said glycosylated product out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium and/or cell extract together and separately can then be further used for separating said glycosylated product.

This preferably involves clarifying said glycosylated product to remove suspended particulates and contaminants, particularly cells, cell components, insoluble metabolites and debris produced by culturing the genetically modified cell. In this step, said glycosylated product can be clarified in a conventional manner. Preferably, said glycosylated product is clarified by centrifugation, flocculation, decantation and/or filtration. A second step of separating said glycosylated product preferably involves removing substantially all the proteins, as well as peptides, amino acids, RNA and DNA and any endotoxins and glycolipids that could interfere with the subsequent separation step, from said glycosylated product, preferably after it has been clarified. In this step, proteins and related impurities can be removed from said glycosylated product in a conventional manner. Preferably, proteins, salts, by-products, colour and other related impurities are removed from said glycosylated product by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane.

Another embodiment of the invention provides for a method and a cell wherein a glycosylated product is produced in and/or by a microorganism, preferably said microorganism is a bacterium. The bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia co/i. The latter bacterium preferably relates to any strain belonging to the species Escherichia coli such as but not limited to Escherichia coli B, Escherichia coli C, Escherichia coli W, Escherichia coli KU, Escherichia coli Nissle. More specifically, the latter term relates to cultivated Escherichia coli strains—designated as E. coli KU strains—which are well-adapted to the laboratory environment, and, unlike wild type strains, have lost their ability to thrive in the intestine. Well-known examples of the E. coli KU strains are KU Wild type, W3110, MG1655, M182, MC1000, MC1060, MC1061, MC4100, JM101, NZN111 and AA200. Hence, the present invention specifically relates to a mutated and/or transformed Escherichia coli cell or strain as indicated above wherein said E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae.

In a preferred embodiment of the method of the present invention, the cell as used herein is cultivated in culture medium comprising sucrose or sucrose combined with another carbon source comprising a monosaccharide, disaccharide, oligosaccharide, polysaccharide, polyol, a complex medium including molasses, corn steep liquor, peptone, tryptone or yeast extract. In a more preferred embodiment of the method of the present invention, said other carbon source is chosen from the list comprising glucose, glycerol, fructose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.

In a further preferred embodiment of the method and/or cell of the present invention, the cell as used herein has improved growth rate on sucrose and/or has improved productivity of the glycosylated product compared to a cell lacking the sucrose permeases from present invention or compared to a cell expressing the sucrose permease cscB from E. coli W with SEQ ID NO 01. Improved growth rate on sucrose should be understood as faster growth on sucrose. Preferably, said growth rate of the cell of present invention is improved when the cell is grown on culture medium comprising a low concentration of sucrose, preferably comprising a concentration of 20 g/L sucrose or below 20 g sucrose/L medium, comprising 19 g/L, 18 g/L, 17 g/L, 16 g/L, 15 g/L, 14 g/L, 13 g/L, 12 g/L, 11 g/L, 10 g/L, 9.0 g/L, 8.0 g/L, 7.0 g/L, 6.0 g/L, 5.0 g/L, 4.0 g/L, 3.0 g/L, 2.0 g/L and 1.0 g/L sucrose. Improved productivity of the glycosylated product should be understood as higher titers (g/L) of glycosylated product produced and/or yield of glycosylated product on sucrose (g glycosylated product/g sucrose consumed) obtained by a cell or the method of present invention.

In a further preferred embodiment, the methods as described herein also provide for a further purification of the glycosylated product. A further purification of said glycosylated product may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product. Another purification step is to dry, e.g. spray dry or lyophilize the produced glycosylated product.

In an exemplary embodiment, the separation and purification of the glycosylated product is made in a process, comprising the following steps in any order:
a) contacting the cultivation or a clarified version thereof with a nanofiltration membrane with a molecular weight cut-off (MWCO) of 600-3500 Da ensuring the retention of the produced glycosylated product and allowing at least a part of the proteins, salts, by-products, colour and other related impurities to pass,
b) conducting a diafiltration process on the retentate from step a), using said membrane, with an aqueous solution of an inorganic electrolyte, followed by optional diafiltration with pure water to remove excess of the electrolyte,
c) and collecting the retentate enriched in the glycosylated product in the form of a salt from the cation of said electrolyte.

In an alternative exemplary embodiment, the separation and purification of the glycosylated product is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of the glycosylated product is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of the glycosylated product is made in the following way. The cultivation comprising the produced glycosylated product, biomass, medium components and contaminants, and wherein the purity of the produced glycosylated product in the fermentation broth is <80 percent, is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,

wherein a purified solution comprising the produced glycosylated product at a purity of greater than or equal to 80 percent is provided. Optionally the purified solution is spray dried.

In an alternative exemplary embodiment, the separation and purification of the glycosylated product is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, the present invention provides the produced glycosylated product which is spray-dried to powder, wherein the spray-dried powder contains <15 percent-wt. of water, preferably <10 percent-wt. of water, more preferably <7 percent-wt. of water, most preferably <5 percent-wt. of water.

In a fourth and fifth aspect, the present invention provides for the use of a metabolically engineered cell and the use of a method as described herein for the production of a glycosylated product.

For identification of the glycosylated product produced in the cell as described herein, the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the glycosylated product methods such as, e.g. acid-catalysed hydrolysis, HPLC (high performance liquid chromatography) or GLC (gas-liquid chromatography) (after conversion to alditol acetates) may be used. To determine the glycosidic linkages, the glycosylated product is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the glycosylated product is subjected to enzymatic analysis, e.g. it is contacted with an enzyme that is specific for a particular type of linkage, e.g., beta-galactosidase, or alpha-glucosidase, etc., and NMR may be used to analyse the products.

### Products Comprising the Glycosylated Product

In some embodiments, a glycosylated product produced as described herein is incorporated into a food (e.g., human food or feed), dietary supplement, pharmaceutical ingredient, cosmetic ingredient or medicine. In some embodiments, the glycosylated product is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the glycosylated product being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a glycosylated product produced and/or purified by a process of this specification is orally administered in combination with such microorganism.

Examples of further ingredients for dietary supplements include disaccharides (such as lactose), monosaccharides (such as glucose and galactose), thickeners (such as gum arabic), acidity regulators (such as trisodium citrate), water, skimmed milk, and flavourings.

In some embodiments, the glycosylated product is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, a glycosylated product produced and/or purified by a process in this specification is included in infant formula to provide nutritional benefits similar to those provided by the oligosaccharides in human breast milk. In some embodiments, the glycosylated product is mixed with one or more ingredients of the infant formula. Examples of infant formula ingredients include nonfat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bit, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the glycosylated products concentration in the infant formula is approximately the same concentration as the glycosylated products concentration generally present in human breast milk.

In some embodiments, the glycosylated product is incorporated into a feed preparation, wherein said feed is chosen from the list comprising pet food, animal milk replacer, veterinary product, post weaning feed, or creep feed.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments, the examples and the attached drawings. It goes without saying that the abovementioned features and the features which are still to be explained below can be used not only in the respectively specified combinations, but also in other combinations or on their own, without departing from the scope of the present invention.

The present invention relates to following specific embodiments:
1. A sucrose permease having sucrose permease activity and comprising a polypeptide sequence that has at least 80% overall sequence identity to SEQ ID NO 01 and that
   i) differs from SEQ ID NO 01 by having an amino acid change for proline at position 169 (P169a), for valine at position 316 (V316c) or for phenylalanine at position 371 (F371e) wherein a, c and e can be any amino acid residue excluding histidine for residue a, or
   ii) shares the serine at position 246 of SEQ ID NO 01 and differs from SEQ ID NO 01 by having an amino acid change for tryptophan at position 230 (W230b) or for cysteine at position 327 (C327d) wherein b and d can be any amino acid residue, or
   iii) differs from SEQ ID NO 01 by having at least two amino acid changes chosen from the list comprising proline at position 169 (P169f), tryptophan at position 230 (W230b), valine at position 316 (V316c), cysteine at position 327 (C327d) and phenylalanine at position 371 (F371e) wherein f, b, c, d and e can be any amino acid residue,
   preferably wherein said amino acid residue:
      a is threonine,
      b is leucine,
      c is chosen from the list comprising alanine, cysteine and phenylalanine,
      d is chosen from the list comprising alanine and glycine,
      e is chosen from the list comprising alanine, cysteine, glutamate, glycine, histidine, isoleucine, lysine, methionine, asparagine, glutamine, serine, threonine, valine and tyrosine, or
      f is threonine.
2. A sucrose permease according to embodiment 1, wherein said sucrose permease
   i) comprises a polypeptide sequence according to any one of SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32, or
   ii) is a functional homologue, variant or derivative of any one of SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32, having at least 80% overall sequence identity to the full length of any one of said polypeptides with SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31 or 32 and having sucrose permease activity.
3. A sucrose permease according to embodiment 2, wherein said sucrose permease with SEQ ID NO 26:
   differs from SEQ ID NO 01 by having an amino acid change for valine at position 316 to alanine, for cysteine at position 327 to glycine and for phenylalanine at position 371 to valine as outlined in SEQ ID NO 33, or
   is a functional homologue, variant or derivative of SEQ ID NO 33 having at least 80% overall sequence identity to the full length of said polypeptide with SEQ ID NO 33 and having sucrose permease activity.
4. A sucrose permease according to any one of embodiments 1 to 3, wherein said sucrose permease further differs from SEQ ID NO 01 by having any one or more amino acid changes chosen from the list comprising:
   leucine at position 61 into proline, tryptophan, histidine, phenylalanine, or tyrosine,
   phenylalanine at position 159 into leucine,
   glycine at position 162 into cysteine,
   proline at position 169 into histidine,
   arginine at position 300 into alanine or leucine,
   glutamine at position 353 into histidine,
   truncation of amino acid residues 404 to 415, or
   truncation of amino acid residues 409 to 415.
5. A metabolically engineered cell for production of a glycosylated product, said cell comprising a pathway for production of said glycosylated product, wherein said cell is capable to express at least one sucrose permease which has sucrose permease activity and comprises a polypeptide sequence that differs from SEQ ID NO 01 by having any one or more of the amino acid changes chosen from the list comprising: proline at position 169 (P169f), tryptophan at position 230 (W230b), valine at position 316 (V316c), cysteine at position 327 (C327d) or phenylalanine at position 371 (F371e) wherein f, b, c, d and e can be any amino acid residue, preferably, wherein amino acid residue:
   f is threonine,
   b is leucine,
   c is chosen from the list comprising alanine, cysteine and phenylalanine,
   d is chosen from the list comprising alanine and glycine, or
   e is chosen from the list comprising alanine, cysteine, glutamate, glycine, histidine, isoleucine, lysine, methionine, asparagine, glutamine, serine, threonine, valine and tyrosine.
6. Cell according to embodiment 5, wherein said sucrose permease has at least 80% overall sequence identity to SEQ ID NO 01.
7. Cell according to embodiment 5, wherein said sucrose permease
   i) comprises a polypeptide sequence according to any one of SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62, or
   ii) is a functional homologue, variant or derivative of any one of SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62 having at least 80% overall sequence identity to the full length of any one of said polypeptides with SEQ ID NOs 34, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 41, 42, 43, 44, 45, 46, 47, 48, 49, 50, 51, 52, 53, 54, 55, 56, 57, 58, 59, 60, 61 or 62 and having sucrose permease activity.
8. Cell according to embodiment 7, wherein said sucrose permease with SEQ ID NO 26:
   differs from SEQ ID NO 01 by having an amino acid change for valine at position 316 to alanine, for cysteine at position 327 to glycine and for phenylalanine at position 371 to valine as outlined in SEQ ID NO 33, or is a functional homologue, variant or derivative of SEQ ID NO 33 having at least 80% overall sequence identity to the full length of said polypeptide with SEQ ID NO 33 and having at least 80% overall sequence identity to the full length of said polypeptide with SEQ ID NO 01 and having sucrose permease activity.
9. Cell according to any one of embodiments 5 to 8, wherein said sucrose permease further differs from SEQ ID NO 01 by having any one or more amino acid changes chosen from the list comprising:
   leucine at position 61 into proline, tryptophan, histidine, phenylalanine or tyrosine,
   phenylalanine at position 159 into leucine,
   glycine at position 162 into cysteine,
   proline at position 169 into histidine,
   arginine at position 300 into alanine or leucine,
   glutamine at position 353 into histidine,
   truncation of amino acid residues 404 to 415, or
   truncation of amino acid residues 409 to 415.
10. Cell according to any one of embodiments 5 to 9, wherein said cell is modified with one or more gene expression modules comprising at least one of said sucrose permeases.
11. Cell according to any one of embodiments 5 to 10, wherein said cell is further capable to synthesize a nucleotide-activated sugar to be used in the production of said glycosylated product.
12. Cell according to embodiment 11, wherein said nucleotide-activated sugar is chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-Iyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
13. Cell according to any one of embodiments 5 to 12, wherein said cell is further capable to express at least one glycosyltransferase chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetyl rhamnosyltransferases, UDP-4-amino-4,6-dideoxy-N-acetyl-beta-L-altrosamine transaminases and fucosaminyltransferases, preferably wherein said cell is modified in the expression or activity of at least one of said glycosyltransferases.
14. Cell according to any one of embodiments 5 to 13, wherein said cell is further engineered:
   with at least one other gene encoding for an enzyme enabling said cell to split sucrose into an activated saccharide and a monosaccharide, and
   wherein at least one gene encoding for an enzyme which converts said activated saccharide into biomass and/or bio-catalytic enzymes is rendered less-functional or non-functional.
15. Cell according to embodiment 14, wherein said enzyme to split sucrose into an activated saccharide and a monosaccharide is chosen from the list comprising sucrose phosphorylases, sucrose synthases, sucrases (invertases) combined with a glucokinase and/or a fructokinase, a trehalase combined with a glucokinase, a maltase combined with a glucokinase, a sucrose-6-phosphate hydrolase combined with a fructokinase, a maltose phosphorylase, a maltose synthase, an amylase combined with a phosphorylase or synthase or hydrolase, a lactose synthase, a lactose phosphorylase, a lactase (or beta-galactosidase) combined with a galactokinase and/or a glucokinase.
16. Cell according to embodiment 14, wherein said other gene which encodes for an enzyme which converts said activated saccharide into biomass and/or bio-catalytic enzymes is chosen from the list comprising genes encoding beta-galactosidase, phosphoglucomutase, glucose-1-phosphate adenylyltransferase, phosphatase, glucose-1-phosphate uridyltransferase, UDP-glucose-4-epimerase, UDP-glucose:galactose-1-phosphate uridyltransferase, UDP-galactopyranose mutase, UDP-galactose:(glucosyl)lipopolysaccharide-1,6-galactosyltransferase, UDP-galactosyltransferase, UDP-glucosyltransferase, UDP-glucuronate transferase, UDP-glucose lipid carrier transferase, UDP-sugar hydrolase, invertase, maltase, trehalase, sugar transporting phosphotransferase, hexokinase.
17. Cell according to any one of embodiments 5 to 16, wherein said cell is a microorganism, preferably said microorganism is a bacterium, preferably of an Escherichia coli strain, more preferably of an Escherichia coli strain which is a K-12 strain, even more preferably the Escherichia coli K-12 strain is E. coli MG1655.
18. Cell according to any one of embodiments 5 to 17, wherein said glycosylated product is chosen from the list comprising:
   di- or an oligosaccharides, nucleosides, glycosides and glycolipids,
   preferably, mammalian milk di- or oligosaccharides,
   more preferably, human milk di- or oligosaccharides.
19. A method to produce a glycosylated product by a cell, the method comprising the steps of:
   i) providing a cell according to any one of embodiments 5 to 18,
   ii) cultivating said cell under conditions permissive to produce said glycosylated product,
   iii) preferably, separating said glycosylated product from said cultivation.
20. Method according to embodiment 19, wherein said cell is cultivated in culture medium comprising sucrose or sucrose combined with another carbon source comprising a monosaccharide, disaccharide, oligosaccharide, polysaccharide, polyol, a complex medium including molasses, corn steep liquor, peptone, tryptone or yeast extract; preferably, wherein said other carbon source is chosen from the list comprising glucose, glycerol, fructose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.
21. Method according to any one of embodiments 19 or 20, wherein said separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.
22. Method according to any one of embodiments 19 to 21, further comprising purification of said glycosylated product from said cell.
23. Method according to embodiment 22, wherein said purification comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.
24. Use of a sucrose permease according to any one of embodiments 1 to 4 for the production of a glycosylated product.
25. Use of a cell according to any one of embodiments 5 to 18 for the production of a glycosylated product.
26. Use of a method according to any one of embodiments 19 to 23 for the production of a glycosylated product.

Moreover, the present invention relates to the following preferred specific embodiments:
1. A sucrose permease having sucrose permease activity and comprising a polypeptide sequence that has at least 80%, preferably at least 85%, more preferably at least 90%, even more preferably at least 95%, most preferably at least 97%, overall sequence identity to SEQ ID NO 01 and that
   i) differs from SEQ ID NO 01 by having an amino acid change for proline at position 169 (P169a), for valine at position 316 (V316c) or for phenylalanine at position 371 (F371e) wherein a, c and e can be any amino acid residue excluding histidine for residue a, or
   ii) shares the serine at position 246 of SEQ ID NO 01 and differs from SEQ ID NO 01 by having an amino acid change for tryptophan at position 230 (W230b) or for cysteine at position 327 (C327d) wherein b and d can be any amino acid residue, or
   iii) differs from SEQ ID NO 01 by having at least two amino acid changes chosen from the list comprising proline at position 169 (P169f), tryptophan at position 230 (W230b), valine at position 316 (V316c), cysteine at position 327 (C327d) and phenylalanine at position 371 (F371e), preferably at least (i) V316c and C327d or (ii) V316c and F371e or (iii) C327d and F371e, more preferably at least C327d and F371e, wherein f, b, c, d and e can be any amino acid residue, or
   iv) differs from SEQ ID NO 01 by having three amino acid changes chosen from the list comprising proline at position 169 (P169f), tryptophan at position 230 (W230b), valine at position 316 (V316c), cysteine at position 327 (C327d) and phenylalanine at position 371 (F371e), preferably at least V316c, C327d and F371e, wherein f, b, c, d and e can be any amino acid residue,
   v) differs from SEQ ID NO 01 by having four amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue,
   vi) differs from SEQ ID NO 01 by having five amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue,
   preferably wherein said amino acid residue:
      a is threonine,
      b is leucine,
      c is chosen from the list consisting of alanine, cysteine and phenylalanine,
      d is chosen from the list consisting of alanine and glycine,
      e is chosen from the list consisting of alanine, cysteine, glutamate, glycine, histidine, isoleucine, lysine, methionine, asparagine, glutamine, serine, threonine, valine and tyrosine, and/or
      f is threonine.
2. A sucrose permease according to preferred embodiment 1, wherein said sucrose permease:
   i) differs from SEQ ID NO 01 by having an amino acid change for proline at position 169 (P169a), for valine at position 316 (V316c) or for phenylalanine at position 371 (F371e) wherein a and e can be any amino acid residue excluding histidine for residue a, and wherein c can be any amino acid residue excluding methionine, or
   ii) shares the serine at position 246 of SEQ ID NO 01 and differs from SEQ ID NO 01 by having an amino acid change for tryptophan at position 230 (W230b) or for cysteine at position 327 (C327d) wherein b and d can be any amino acid residue, or
   iii) differs from SEQ ID NO 01 by having at least two amino acid changes chosen from the list comprising proline at position 169 (P169f), tryptophan at position 230 (W230b), valine at position 316 (V316c), cysteine at position 327 (C327d) and phenylalanine at position 371 (F371e), preferably at least (i) V316c and C327d or (ii) V316c and F371e or (iii) C327d and F371e, more preferably at least C327d and F371e, wherein f, b, c, d and e can be any amino acid residue, preferably wherein c can be any amino acid residue excluding methionine,
   iv) differs from SEQ ID NO 01 by having three amino acid changes chosen from the list comprising proline at position 169 (P169f), tryptophan at position 230 (W230b), valine at position 316 (V316c), cysteine at position 327 (C327d) and phenylalanine at position 371 (F371e), preferably at least V316c, C327d and F371e, wherein f, b, c, d and e can be any amino acid residue, preferably wherein c can be any amino acid residue excluding methionine,
   v) differs from SEQ ID NO 01 by having four amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue, preferably wherein c can be any amino acid residue excluding methionine,
   vi) differs from SEQ ID NO 01 by having five amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue, preferably wherein c can be any amino acid residue excluding methionine,
   preferably wherein said amino acid residue:
      a is threonine,
      b is leucine,
      c is chosen from the list consisting of alanine, cysteine and phenylalanine,
      d is chosen from the list consisting of alanine and glycine,
      e is chosen from the list consisting of alanine, cysteine, glutamate, glycine, histidine, isoleucine, lysine, methionine, asparagine, glutamine, serine, threonine, valine and tyrosine, and/or
      f is threonine.
3. A sucrose permease according to preferred embodiment 1, wherein said sucrose permease:
   i) differs from SEQ ID NO 01 by having an amino acid change for proline at position 169 (P169a), for valine at position 316 (V316c) or for phenylalanine at position 371 (F371e) wherein a and e can be any amino acid residue excluding histidine for residue a, and wherein c is chosen from the list consisting of alanine, cysteine and phenylalanine, or
   ii) shares the serine at position 246 of SEQ ID NO 01 and differs from SEQ ID NO 01 by having an amino acid change for tryptophan at position 230 (W230b) or for cysteine at position 327 (C327d) wherein b and d can be any amino acid residue, or
   iii) differs from SEQ ID NO 01 by having at least two amino acid changes chosen from the list comprising proline at position 169 (P169f), tryptophan at position 230 (W230b), valine at position 316 (V316c), cysteine at position 327 (C327d) and phenylalanine at position 371 (F371e), preferably at least (i) V316c and C327d or (ii) V316c and F371e or (iii) C327d and F371e, more preferably at least C327d and F371e, wherein f, b, c, d and e can be any amino acid residue,
   iv) differs from SEQ ID NO 01 by having at least three amino acid changes chosen from the list comprising proline at position 169 (P169f), tryptophan at position 230 (W230b), valine at position 316 (V316c), cysteine at position 327 (C327d) and phenylalanine at position 371 (F371e), preferably at least V316c, C327d and F371e, wherein f, b, c, d and e can be any amino acid residue,
   v) differs from SEQ ID NO 01 by having four amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue,
   vi) differs from SEQ ID NO 01 by having five amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue,
   preferably wherein said amino acid residue:
      a is threonine,
      b is leucine,
      c is chosen from the list consisting of alanine, cysteine and phenylalanine,
      d is chosen from the list consisting of alanine and glycine,
      e is chosen from the list consisting of alanine, cysteine, glutamate, glycine, histidine, isoleucine, lysine, methionine, asparagine, glutamine, serine, threonine, valine and tyrosine, and/or
      f is threonine.
4. A sucrose permease according to any one of preferred embodiments 1 to 3, wherein said sucrose permease has an improved affinity for sucrose compared to the sucrose permease represented with SEQ ID NO 1.
5. A sucrose permease according to any one of preferred embodiments 1 to 4, wherein said sucrose permease
   i) comprises a polypeptide sequence according to any one of SEQ ID NOs 26, 05, 16, 02, 03, 04, 06, 07, 08, 09, 10, 11, 12, 13, 14, 15, 17, 18, 19, 20, 21, 22, 23, 24, 25, 27, 28, 29, 30, 31 or 32, or
   ii) is a functional homologue, variant or derivative of any one of SEQ ID NOs 26, 05, 16, 02, 03, 04, 06, 07, 08, 09, 10, 11, 12, 13, 14, 15, 17, 18, 19, 20, 21, 22, 23, 24, 25, 27, 28, 29, 30, 31 or 32, having at least 80%, preferably at least 85%, more preferably at least 90%, even more preferably at least 95%, most preferably at least 97%, overall sequence identity to the full length of any one of said polypeptides with SEQ ID NOs 26, 05, 16, 02, 03, 04, 06, 07, 08, 09, 10, 11, 12, 13, 14, 15, 17, 18, 19, 20, 21, 22, 23, 24, 25, 27, 28, 29, 30, 31 or 32 and having sucrose permease activity.
6. A sucrose permease according to any one of preferred embodiments 1 to 5, wherein said sucrose permease
   i) comprises a polypeptide sequence according to SEQ ID NO 32, or
   ii) is a functional homologue, variant or derivative of SEQ ID NO 32, having at least 80%, preferably at least 85%, more preferably at least 90%, even more preferably at least 95%, most preferably at least 97%, overall sequence identity to the full length of polypeptides with SEQ ID NO 32 and having sucrose permease activity, preferably wherein said functional homologue, variant or derivative differ from SEQ ID NO 01 by having five amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue.
7. A sucrose permease according to any one of preferred embodiments 1 to 5, wherein said sucrose permease
   i) comprises a polypeptide sequence according to SEQ ID NOs 27, 28, 29, 30 or 31, or
   ii) is a functional homologue, variant or derivative of any one of SEQ ID NOs 27, 28, 29, 30 or 31, having at least 80%, preferably at least 85%, more preferably at least 90%, even more preferably at least 95%, most preferably at least 97%, overall sequence identity to the full length of any one of said polypeptides with SEQ ID NOs 27, 28, 29, 30 or 31 and having sucrose permease activity, preferably wherein said functional homologue, variant or derivative differ from SEQ ID NO 01 by having four amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue.
8. A sucrose permease according to any one of preferred embodiments 1 to 5, wherein said sucrose permease
   i) comprises a polypeptide sequence according to SEQ ID NOs 26, 17, 18, 19, 20, 21, 22, 23, 24 or or
   ii) is a functional homologue, variant or derivative of any one of SEQ ID NOs 26, 17, 18, 19, 20, 21, 22, 23, 24 or 25, having at least 80%, preferably at least 85%, more preferably at least 90%, even more preferably at least 95%, most preferably at least 97%, overall sequence identity to the full length of any one of said polypeptides with SEQ ID NOs 26, 17, 18, 19, 20, 21, 22, 23, 24 or 25 and having sucrose permease activity, preferably wherein said functional homologue, variant or derivative differ from SEQ ID NO 01 by having three amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue.
9. A sucrose permease according to preferred embodiment 5 or 8, wherein said sucrose permease with SEQ ID NO 26:
   differs from SEQ ID NO 01 by having an amino acid change for valine at position 316, for cysteine at position 327 and for phenylalanine at position 371, preferably as outlined in SEQ ID NO 33, 110, 111, 112, 113 or 114, more preferably as outlined in SEQ ID NO 33, or
   is a functional homologue, variant or derivative of any one of SEQ ID NOs 33, 110, 111, 112, 113 or 114, preferably SEQ ID NO 33, having at least 80%, preferably at least 85%, more preferably at least 90%, even more preferably at least 95%, most preferably at least 97%, overall sequence identity to the full length of any one of said polypeptides with SEQ ID NOs 33, 110, 111, 112, 113 or 114, preferably SEQ ID NO 33, and having sucrose permease activity, preferably wherein said functional homologue, variant or derivative differ from SEQ ID NO 01 by having an amino acid change for valine at position 316, for cysteine at position 327 and for phenylalanine at position 371, more preferably wherein said functional homologue, variant or derivative differ from SEQ ID NO 01 by having an amino acid change for valine at position 316 to alanine, isoleucine, methionine, leucine or phenylalanine, for cysteine at position 327 to glycine or alanine and for phenylalanine at position 371 to valine, threonine, glycine, serine or cysteine, most preferably wherein said functional homologue, variant or derivative differ from SEQ ID NO 01 by having an amino acid change for valine at position 316 to alanine, for cysteine at position 327 to glycine and for phenylalanine at position 371 to valine.
10. A sucrose permease according to any one of preferred embodiments 1 to 5, wherein said sucrose permease
   i) comprises a polypeptide sequence according to SEQ ID NOs 16, 07, 08, 09, 10, 11, 12, 13, 14 or 15, or
   ii) is a functional homologue, variant or derivative of any one of SEQ ID NOs 16, 07, 08, 09, 10, 11, 12, 13, 14 or 15, having at least 80%, preferably at least 85%, more preferably at least 90%, even more preferably at least 95%, most preferably at least 97%, overall sequence identity to the full length of any one of said polypeptides with SEQ ID NOs 16, 07, 08, 09, 10, 11, 12, 13, 14 or 15 and having sucrose permease activity, preferably wherein said functional homologue, variant or derivative differ from SEQ ID NO 01 by having two amino acid changes chosen from the list comprising proline at position 169 (P169f), tryptophan at position 230 (W230b), valine at position 316 (V316c), cysteine at position 327 (C327d) and phenylalanine at position 371 (F371e), wherein f, b, c, d and e can be any amino acid residue.
11. A sucrose permease according to preferred embodiment 5 or 10, wherein said sucrose permease with SEQ ID NO 16:
   differs from SEQ ID NO 01 by having an amino acid change for cysteine at position 327 and for phenylalanine at position 371, preferably as outlined in SEQ ID NO 40, 117 or 118, more preferably as outlined in SEQ ID NO 40, or
   is a functional homologue, variant or derivative of any one of SEQ ID NOs 40, 117 or 118, preferably SEQ ID NO 40, having at least 80%, preferably at least 85%, more preferably at least 90%, even more preferably at least 95%, most preferably at least 97%, overall sequence identity to the full length of any one of said polypeptides with SEQ ID NOs 40, 117 or 118, preferably SEQ ID NO 40, and having sucrose permease activity, preferably wherein said functional homologue, variant or derivative differ from SEQ ID NO 01 by having an amino acid change for cysteine at position 327 and for phenylalanine at position 371, more preferably wherein said functional homologue, variant or derivative differ from SEQ ID NO 01 by having an amino acid change for cysteine at position 327 to glycine and for phenylalanine at position 371 to valine.
12. A sucrose permease according to preferred embodiment 5 or 10, wherein said sucrose permease with SEQ ID NO 14:
   differs from SEQ ID NO 01 by having an amino acid change for valine at position 316 and for cysteine at position 327, preferably as outlined in SEQ ID NO 38, or
   is a functional homologue, variant or derivative of SEQ ID No 38, having at least 80%, preferably at least 85%, more preferably at least 90%, even more preferably at least 95%, most preferably at least 97%, overall sequence identity to the full length of said polypeptide with SEQ ID NO 38 and having sucrose permease activity, preferably wherein said functional homologue, variant or derivative differ from SEQ ID NO 01 by having an amino acid change for valine at position 316 and for cysteine at position 327, more preferably wherein said functional homologue, variant or derivative differ from SEQ ID NO 01 by having an amino acid change for valine at position 316 to alanine and for cysteine at position 327 to glycine.
13. A sucrose permease according to preferred embodiment 5 or 10, wherein said sucrose permease with SEQ ID NO 15:
   differs from SEQ ID NO 01 by having an amino acid change for valine at position 316 and for phenylalanine at position 371, preferably as outlined in SEQ ID NO 39, or
   is a functional homologue, variant or derivative of SEQ ID No 39, having at least 80%, preferably at least 85%, more preferably at least 90%, even more preferably at least 95%, most preferably at least 97%, overall sequence identity to the full length of said polypeptide with SEQ ID NO 39 and having sucrose permease activity, preferably wherein said functional homologue, variant or derivative differ from SEQ ID NO 01 by having an amino acid change for valine at position 316 and for phenylalanine at position 371, more preferably wherein said functional homologue, variant or derivative differ from SEQ ID NO 01 by having an amino acid change for valine at position 316 to alanine and for phenylalanine at position 371 to valine.
14. A sucrose permease according to any one of preferred embodiments 1 to 5, wherein said sucrose permease
   i) comprises a polypeptide sequence according to SEQ ID NOs 05, 02, 03, 04 or 06, or
   ii) is a functional homologue, variant or derivative of any one of SEQ ID NOs 05, 02, 03, 04 or 06, having at least 80%, preferably at least 85%, more preferably at least 90%, even more preferably at least 95%, most preferably at least 97%, overall sequence identity to the full length of any one of said polypeptides with SEQ ID NOs 05, 02, 03, 04 or 06 and having sucrose permease activity, preferably wherein said functional homologue, variant or derivative differs from SEQ ID NO 01 by having an amino acid change chosen from the list comprising proline at position 169 (P169a), tryptophan at position 230 (W230b), valine at position 316 (V316c), cysteine at position 327 (C327d) and phenylalanine at position 371 (F371e), wherein a, b, c, d and e can be any amino acid residue excluding histidine for residue a.
15. A sucrose permease according to preferred embodiment 5 or 14, wherein said sucrose permease with SEQ ID NO 05:
   differs from SEQ ID NO 01 by having an amino acid change for cysteine at position 327, preferably as outlined in SEQ ID NO 36 or 100, more preferably as outlined in SEQ ID NO 36, or
   is a functional homologue, variant or derivative of SEQ ID No 36 or 100, preferably SEQ ID 36, having at least 80%, preferably at least 85%, more preferably at least 90%, even more preferably at least 95%, most preferably at least 97%, overall sequence identity to the full length of said polypeptides with SEQ ID NO 36 or 100, preferably SEQ ID NO 36, and having sucrose permease activity, preferably wherein said functional homologue, variant or derivative differ from SEQ ID NO 01 by having an amino acid change for cysteine at position 327, more preferably wherein said functional homologue, variant or derivative differ from SEQ ID NO 01 by having an amino acid change for cysteine at position 327 to glycine or alanine, most preferably wherein said functional homologue, variant or derivative differ from SEQ ID NO 01 by having an amino acid change for cysteine at position 327 to glycine.
16. A sucrose permease according to preferred embodiment 5 or 14, wherein said sucrose permease with SEQ ID NO 04:
   differs from SEQ ID NO 01 by having an amino acid change for valine at position 316, preferably as outlined in SEQ ID NOs 35, 98 or 99, more preferably as outlined in SEQ ID NO 35, or
   is a functional homologue, variant or derivative of any one of SEQ ID NOs 35, 98 or 99, preferably SEQ ID 35, having at least 80%, preferably at least 85%, more preferably at least 90%, even more preferably at least 95%, most preferably at least 97%, overall sequence identity to the full length of any one of said polypeptides with SEQ ID NOs 35, 98 or 99, preferably SEQ ID NO 35, and having sucrose permease activity, preferably wherein said functional homologue, variant or derivative differ from SEQ ID NO 01 by having an amino acid change for valine at position 316, more preferably wherein said functional homologue, variant or derivative differ from SEQ ID NO 01 by having an amino acid change for valine at position 316 to alanine, cysteine or phenylalanine, most preferably wherein said functional homologue, variant or derivative differ from SEQ ID NO 01 by having an amino acid change for valine at position 316 to alanine.
17. A sucrose permease according to preferred embodiment 5 or 14, wherein said sucrose permease with SEQ ID NO 06:
   differs from SEQ ID NO 01 by having an amino acid change for phenylalanine at position 371, preferably as outlined in SEQ ID NOs 37, 101, 102, 103, 104, 105, 106, 107, 108, 109, 119, 120 or 121, more preferably as outlined in SEQ ID NO 37, or
   is a functional homologue, variant or derivative of any one of SEQ ID NOs 37, 101, 102, 103, 104, 105, 106, 107, 108, 109, 119, 120 or 121, preferably SEQ ID 37, having at least 80%, preferably at least 85%, more preferably at least 90%, even more preferably at least 95%, most preferably at least 97%, overall sequence identity to the full length of any one of said polypeptides with SEQ ID NOs 37, 101, 102, 103, 104, 105, 106, 107, 108, 109, 119, 120 or 121, preferably SEQ ID NO 37, and having sucrose permease activity, preferably wherein said functional homologue, variant or derivative differ from SEQ ID NO 01 by having an amino acid change for phenylalanine at position 371, more preferably wherein said functional homologue, variant or derivative differ from SEQ ID NO 01 by having an amino acid change for phenylalanine at position 371 to valine, tyrosine, glutaminic acid, glutamine, leucine, alanine, threonine, cysteine, serine, isoleucine, glycine, methionine or asparagine, most preferably wherein said functional homologue, variant or derivative differ from SEQ ID NO 01 by having an amino acid change for phenylalanine at position 371 to valine.
18. A sucrose permease according to any one of preferred embodiments 1 to 17, wherein said sucrose permease further differs from SEQ ID NO 01 by having any one or more amino acid changes chosen from the list comprising:
   leucine at position 61 into proline, tryptophan, histidine, phenylalanine, or tyrosine,
   phenylalanine at position 159 into leucine,
   glycine at position 162 into cysteine,
   proline at position 169 into histidine,
   arginine at position 300 into alanine or leucine,
   glutamine at position 353 into histidine,
   truncation of amino acid residues 404 to 415, or
   truncation of amino acid residues 409 to 415.
19. A sucrose permease according to preferred embodiment 18, wherein said sucrose permease is:
   represented by SEQ ID NO: 118 or 119, or
   is a functional homologue, variant or derivative of SEQ ID NO 118 or 119, having at least 80%, preferably at least 85%, more preferably at least 90%, even more preferably at least 95%, most preferably at least 97%, overall sequence identity to the full length of said polypeptides with SEQ ID NO 118 or 119 and having sucrose permease activity, preferably wherein said functional homologue, variant or derivative differ from SEQ ID NO 01 by having an amino acid change for cysteine at position 327 to glycine and for phenylalanine at position 371 for valine.
20. A sucrose permease according to any one of preferred embodiments 1 to 19, wherein said sucrose permease comprises of at least 350, preferably at least 360, more preferably at least 370, even more preferably at least 380, even more preferably at least 390, most preferably at least 400 amino acids.
21. A sucrose permease according to any one of preferred embodiments 1 to 20, wherein said sucrose permease comprises of less than 450, preferably less than 440, more preferably less than 430 amino acids.
22. A metabolically engineered cell for production of a glycosylated product, said cell comprising a pathway for production of said glycosylated product, wherein said cell is capable to express at least one sucrose permease which has sucrose permease activity and comprises a polypeptide sequence that differs from SEQ ID NO 01 by (i) having any one or more of the amino acid changes chosen from the list comprising: proline at position 169 (P169f), tryptophan at position 230 (W230b), valine at position 316 (V316c), cysteine at position 327 (C327d) or phenylalanine at position 371 (F371e) wherein f, b, c, d and e can be any amino acid residue, preferably, wherein amino acid residue:
   f is threonine,
   b is leucine,
   c is chosen from the list comprising alanine, cysteine and phenylalanine,
   d is chosen from the list comprising alanine and glycine, and/or
   e is chosen from the list comprising alanine, cysteine, glutamate, glycine, histidine, isoleucine, lysine, methionine, asparagine, glutamine, serine, threonine, valine and tyrosine,
   and/or by (ii) a truncation of amino acid residues 403 to 415, 404 to 415, 405 to 415, 406 to 415, 407 to 415, 408 to 415, 409 to 415, 410 to 415, 411 to 415, 412 to 415, 413 to 415 or 414 to 415, preferably by a truncation of amino acid residues 403 to 415, 404 to 415, 405 to 415, 406 to 415, 407 to 415, 408 to 415, 409 to 415 or 410 to 415, more preferably by a truncation of amino acid residues 403 to 415, 404 to 415 or 409 to 415, most preferably by a truncation of amino acid residues 404 to 415 or 409 to 415,
   preferably wherein said cell is metabolically engineered for the production of said glycosylated product, more preferably wherein said cell is metabolically engineered to comprise a pathway for production of said glycosylated product.
23. Cell according to preferred embodiment 22 wherein said sucrose permease has an improved affinity for sucrose compared to the sucrose permease represented with SEQ ID NO 01.
24. Cell according to preferred embodiment 22 or 23, wherein said sucrose permease has at least 80%, preferably at least 85%, more preferably at least 90%, even more preferably at least 95%, most preferably at least 97%, overall sequence identity to SEQ ID NO 01.
25. Cell according to any one of preferred embodiments 22 to 24, wherein said sucrose permease
   i) comprises a polypeptide sequence according to any one of SEQ ID NOs 26, 05, 16, 34, 03, 04, 06, 07, 08, 09, 10, 11, 12, 13, 14, 15, 17, 18, 19, 20, 21, 22, 23, 24, 25, 27, 28, 29, 30, 31, 32, 41, 42, 43, 44, 45, 46, 47, 48, 49, 50, 51, 52, 53, 54, 55, 56, 57, 58, 59, 60, 61, 62, 35, 36, 37, 38, 39, 40, 98, 99, 100, 101, 102, 103, 104, 105, 106, 107, 108, 109, 110, 111, 112, 113, 114, 115, 116, 117, 118, 119, 120 or 121, or
   ii) is a functional homologue, variant or derivative of any one of SEQ ID NOs 26, 05, 16, 34, 03, 04, 06, 07, 08, 09, 10, 11, 12, 13, 14, 15, 17, 18, 19, 20, 21, 22, 23, 24, 25, 27, 28, 29, 30, 31, 32, 41, 42, 43, 44, 45, 46, 47, 48, 49, 50, 51, 52, 53, 54, 55, 56, 57, 58, 59, 60, 61, 62, 35, 36, 37, 38, 39, 98, 99, 100, 101, 102, 103, 104, 105, 106, 107, 108, 109, 110, 111, 112, 113, 114, 115, 116, 117, 118, 119, 120 or 121, having at least 80%, preferably at least 85%, more preferably at least %, even more preferably at least 95%, most preferably at least 97%, overall sequence identity to the full length of any one of said polypeptides with SEQ ID NOs 26, 05, 16, 34, 03, 04, 06, 07, 08, 09, 10, 11, 12, 13, 14, 15, 17, 18, 19, 20, 21, 22, 23, 24, 25, 27, 28, 29, 30, 31, 32, 41, 42, 43, 44, 45, 46, 47, 48, 49, 50, 51, 52, 53, 54, 55, 56, 57, 58, 59, 60, 61, 62, 35, 36, 37, 38, 39, 40, 98, 99, 100, 101, 102, 103, 104, 105, 106, 107, 108, 109, 110, 111, 112, 113, 114, 115, 116, 117, 118, 119, 120 or 121 and having sucrose permease activity.
26. Cell according to preferred embodiment 25, wherein said sucrose permease with SEQ ID NO 26:
   differs from SEQ ID NO 01 by having an amino acid change for valine at position 316, for cysteine at position 327 and for phenylalanine at position 371, preferably as outlined in SEQ ID NO 33, 110, 111, 112, 113 or 114, more preferably as outlined in SEQ ID NO 33, or
   is a functional homologue, variant or derivative of any one of SEQ ID NOs 33, 110, 111, 112, 113 or 114, preferably SEQ ID NO 33, having at least 80%, preferably at least 85%, more preferably at least 90%, even more preferably at least 95%, most preferably at least 97%, overall sequence identity to the full length of any one of said polypeptides with SEQ ID NOs 33, 110, 111, 112, 113 or 114, preferably SEQ ID NO 33, and having at least 80%, preferably at least 85%, more preferably at least 90%, even more preferably at least 95%, most preferably at least 97%, overall sequence identity to the full length of said polypeptide with SEQ ID NO 01 and having sucrose permease activity, preferably wherein said functional homologue, variant or derivative differ from SEQ ID NO 01 by having an amino acid change for valine at position 316, for cysteine at position 327 and for phenylalanine at position 371, more preferably wherein said functional homologue, variant or derivative differ from SEQ ID NO 01 by having an amino acid change for valine at position 316 to alanine, isoleucine, methionine, leucine or phenylalanine, for cysteine at position 327 to glycine or alanine and for phenylalanine at position 371 to valine, threonine, glycine, serine or cysteine, most preferably wherein said functional homologue, variant or derivative differ from SEQ ID NO 01 by having an amino acid change for valine at position 316 to alanine, for cysteine at position 327 to glycine and for phenylalanine at position 371 to valine.
27. Cell according to any one of preferred embodiments 22 to 26, wherein said sucrose permease further differs from SEQ ID NO 01 by having any one or more amino acid changes chosen from the list comprising:
   leucine at position 61 into proline, tryptophan, histidine, phenylalanine or tyrosine,
   phenylalanine at position 159 into leucine,
   glycine at position 162 into cysteine,
   proline at position 169 into histidine,
   arginine at position 300 into alanine or leucine,
   glutamine at position 353 into histidine,
   truncation of amino acid residues 404 to 415, or
   truncation of amino acid residues 409 to 415.
28. A metabolically engineered cell for production of a glycosylated product, said cell comprising a pathway for production of said glycosylated product, wherein said cell is capable to express at least one sucrose permease according to any one of preferred embodiments 1 to 21.
29. Cell according to any one of preferred embodiments 22 to 28, wherein said cell is modified with one or more gene expression modules comprising at least one of said sucrose permeases.
30. Cell according to any one of preferred embodiments 22 to 29, wherein said cell is further capable to synthesize a nucleotide-activated sugar to be used in the production of said glycosylated product.
31. Cell according to preferred embodiment 30, wherein said nucleotide-activated sugar is chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
32. Cell according to any one of preferred embodiments 22 to 31, wherein said cell is further capable to express at least one glycosyltransferase chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases and fucosaminyltransferases, preferably wherein said cell is modified in the expression or activity of at least one of said glycosyltransferases.
33. Cell according to any one of preferred embodiments 22 to 32, wherein said cell is further engineered:
   with at least one other gene encoding for an enzyme enabling said cell to split sucrose into an activated saccharide and a monosaccharide, and
   wherein at least one gene encoding for an enzyme which converts said activated saccharide into biomass and/or bio-catalytic enzymes is rendered less-functional or non-functional.
34. Cell according to preferred embodiment 33, wherein said enzyme to split sucrose into an activated saccharide and a monosaccharide is chosen from the list comprising sucrose phosphorylases, sucrose synthases, sucrases (invertases) combined with a glucokinase and/or a fructokinase, a trehalase combined with a glucokinase, a maltase combined with a glucokinase, a sucrose-6-phosphate hydrolase combined with a fructokinase, a maltose phosphorylase, a maltose synthase, an amylase combined with a phosphorylase or synthase or hydrolase, a lactose synthase, a lactose phosphorylase, a lactase (or beta-galactosidase) combined with a galactokinase and/or a glucokinase.
35. Cell according to preferred embodiment 33 or 34, wherein said other gene which encodes for an enzyme which converts said activated saccharide into biomass and/or bio-catalytic enzymes is chosen from the list comprising genes encoding beta-galactosidase, phosphoglucomutase, glucose-1-phosphate adenylyltransferase, phosphatase, glucose-1-phosphate uridyltransferase, UDP-glucose-4-epimerase, UDP-glucose:galactose-1-phosphate uridyltransferase, UDP-galactopyranose mutase, UDP-galactose:(glucosyl)lipopolysaccharide-1,6-galactosyltransferase, UDP-galactosyltransferase, UDP-glucosyltransferase, UDP-glucuronate transferase, UDP-glucose lipid carrier transferase, UDP-sugar hydrolase, invertase, maltase, trehalase, sugar transporting phosphotransferase, hexokinase.
36. Cell according to any one of preferred embodiments 22 to 35, wherein said cell is a microorganism, preferably said microorganism is a bacterium, preferably of an Escherichia coli strain, more preferably of an Escherichia coli strain which is a K-12 strain, even more preferably the Escherichia coli K-12 strain is E. coli MG1655.
37. Cell according to any one of preferred embodiments 22 to 36, wherein said glycosylated product is chosen from the list comprising:
   di- or an oligosaccharides, nucleosides, glycosides and glycolipids,
   preferably, mammalian milk di- or oligosaccharides,
   more preferably, human milk di- or oligosaccharides.
38. A method to produce a glycosylated product by a cell, the method comprising the steps of:
   i) providing a cell according to any one of preferred embodiments 22 to 37,
   ii) cultivating said cell under conditions permissive to produce said glycosylated product,
   iii) preferably, separating said glycosylated product from said cultivation.
39. Method according to preferred embodiment 38, wherein said cell is cultivated in culture medium comprising sucrose or sucrose combined with another carbon source comprising a monosaccharide, disaccharide, oligosaccharide, polysaccharide, polyol, a complex medium including molasses, corn steep liquor, peptone, tryptone or yeast extract; preferably, wherein said other carbon source is chosen from the list comprising glucose, glycerol, fructose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.
40. Method according to preferred embodiment 38 or 39, wherein said separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.
41. Method according to any one of preferred embodiments 38 to 40, further comprising purification of said glycosylated product from said cell.
42. Method according to preferred embodiment 41, wherein said purification comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.
43. Use of a sucrose permease according to any one of preferred embodiments 1 to 21 for the production of a glycosylated product.
44. Use of a cell according to any one of preferred embodiments 22 to 37 for the production of a glycosylated product.
45. Use of a method according to any one of preferred embodiments 38 to 42 for the production of a glycosylated product.

The invention will be described in more detail in the examples and the attached figures, in which

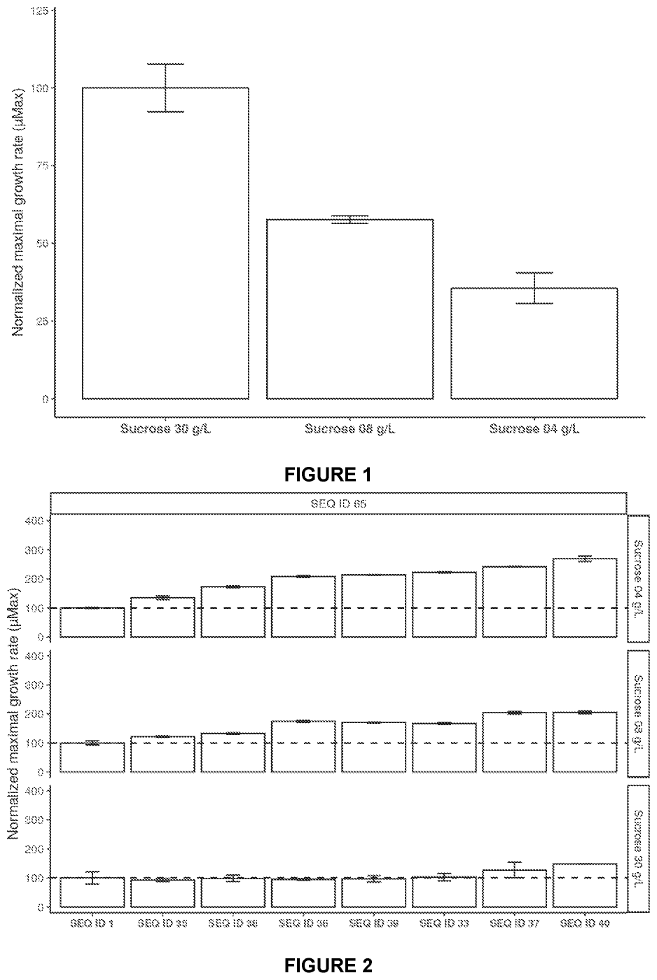

FIG. 1 shows the normalized maximal growth rate (μMax in %) of an E. coli 3-FL production strain comprising the wild type CscB sucrose permease with SEQ ID NO 01 from E. coli W when grown on minimal medium comprising 4, 8 or 30 g/L sucrose.

FIG. 2 shows the normalized maximal growth rate (μMax in %) of E. coli 2′-FL production strains expressing the a1,2-fucosyltransferase with SEQ ID NO 65 from H. pylori and either wild-type CscB from E. coli W with SEQ ID NO 01 or a variant thereof comprising a V316A, C327G and/or F371V mutation as specified in SEQ ID NOs 33, 35, 36, 37, 38, 39, 40, when grown on minimal medium comprising 4, 8 or 30 g/L sucrose.

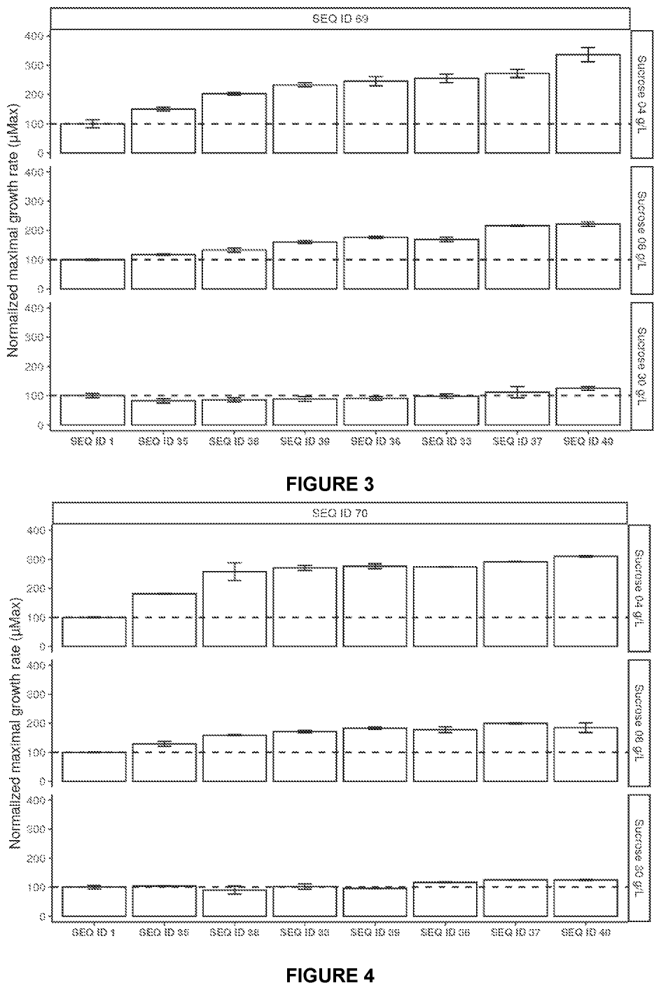

FIG. 3 shows the normalized maximal growth rate (μMax in %) of E. coli 3-FL production strains expressing the a1,3-fucosyltransferase with SEQ ID NO 69 from B. psittacipulmonis and either wild-type CscB from E. coli W with SEQ ID NO 01 or a variant thereof comprising a V316A, C327G and/or F371V mutation as specified in SEQ ID NOs 33, 35, 36, 37, 38, 39, 40, when grown on minimal medium comprising 4, 8 or 30 g/L sucrose.

FIG. 4 shows the normalized maximal growth rate (μMax in %) of E. coli 3-FL production strains expressing the a1,3-fucosyltransferase with SEQ ID NO 70 from A. oryzae and either wild-type CscB from E. coli W with SEQ ID NO 01 or a variant thereof comprising a V316A, C327G and/or F371V mutation as specified in SEQ ID NOs 33, 35, 36, 37, 38, 39, 40, when grown on minimal medium comprising 4, 8 or 30 g/L sucrose.

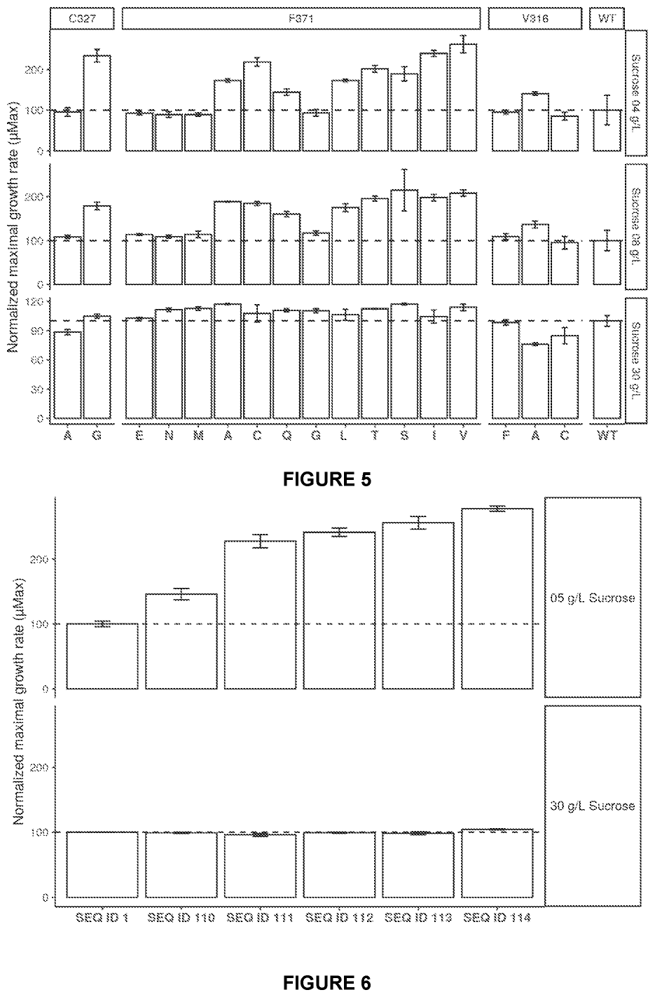

FIG. 5 shows the normalized maximal growth rate (μMax in %) of E. coli 3-FL production strains expressing the a1,3-fucosyltransferase with SEQ ID NO 69 from B. psittacipulmonis and either wild-type CscB from E. coli W with SEQ ID NO 01 (WT) or a variant thereof differing by an amino acid change at position C327 into A (SEQ ID NO 100) or G (SEQ ID NO 36), at position F371 into E (SEQ ID NO 102), N (SEQ ID NO 121), M (SEQ ID NO 120), A (SEQ ID NO 105), C (SEQ ID NO 107), Q (SEQ ID NO 103), G (SEQ ID NO 119), L (SEQ ID NO 104), T (SEQ ID NO 106), S (SEQ ID NO 108), I (SEQ ID NO 109) or V (SEQ ID NO 37), or at position V316 into F (SEQ ID NO 99), A (SEQ ID NO 35) or C (SEQ ID NO 98), when grown on minimal medium comprising 4, 8 or 30 g/L sucrose.

FIG. 6 shows the normalized maximal growth rate (μMax in %) of E. coli 3-FL production strains expressing the a1,3-fucosyltransferase with SEQ ID NO 69 from B. psittacipulmonis and either wild-type CscB from E. coli W with SEQ ID NO 01 (WT) or a variant thereof with either SEQ ID NO 110 (differing from SEQ ID NO 01 by a V3161, C327A, F371T mutation), SEQ ID NO 111 (differing from SEQ ID NO 01 by a V316M, C327G and F371G mutation), SEQ ID NO 112 (differing from SEQ ID NO 01 by a V316L, C327G and F371S mutation), SEQ ID NO 113 (differing from SEQ ID NO 01 by a V316F, C327G and F371V mutation) or SEQ ID NO 114 (differing from SEQ ID NO 01 by a V3161, C327G and F371C mutation), when grown on minimal medium comprising 5 or 30 g/L sucrose.

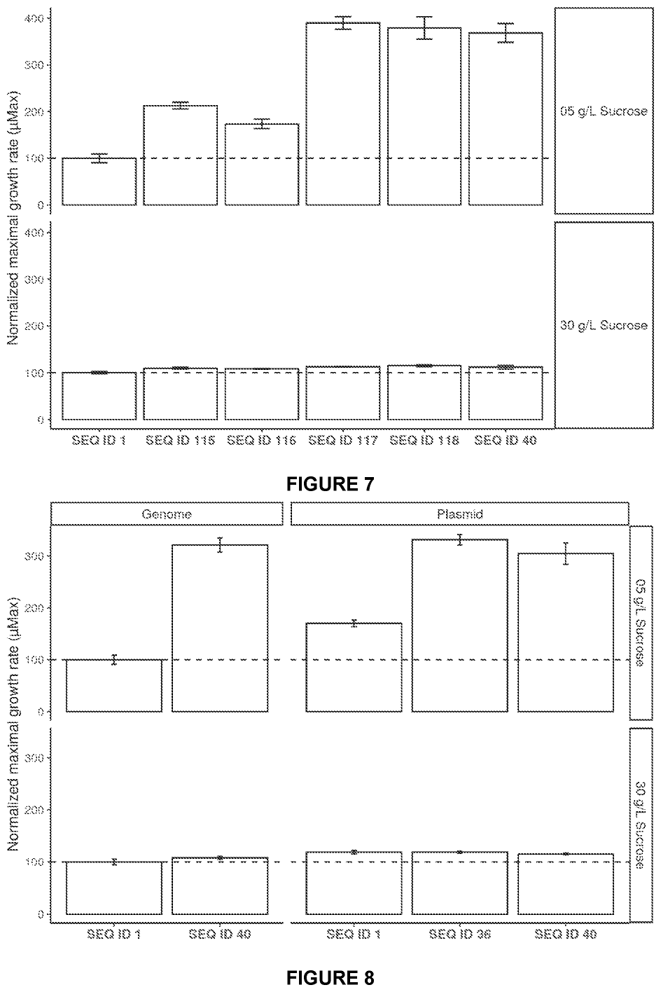

FIG. 7 shows the normalized maximal growth rate (μMax in %) of E. coli 3-FL production strains expressing the a1,3-fucosyltransferase with SEQ ID NO 69 from B. psittacipulmonis and either wild-type CscB from E. coli W with SEQ ID NO 01 (WT) or a variant thereof with either SEQ ID NO 115 (differing from SEQ ID NO 01 by a C-terminal truncation of 12 amino acid residues), SEQ ID NO 116 (differing from SEQ ID NO 01 by a C-terminal truncation of 7 amino acid residues), SEQ ID NO 40 (differing from SEQ ID NO 01 by a C327G and a F371V mutation), SEQ ID NO 117 (differing from SEQ ID NO 01 by a C327G and a F371V mutation and by a C-terminal truncation of 12 amino acid residues) or SEQ ID NO 118 (differing from SEQ ID NO 01 by a C327G and a F371V mutation by a C-terminal truncation of 7 amino acid residues), when grown on minimal medium comprising 5 or 30 g/L sucrose.

FIG. 8 shows the normalized maximal growth rate (μMax in %) of E. coli 3-FL production strains expressing the a1,3-fucosyltransferase with SEQ ID NO 69 from B. psittacipulmonis and either wild-type CscB from E. coli W with SEQ ID NO 01 (WT) or a variant thereof with either SEQ ID NO 36 or 40 from the genome or from an expression plasmid, when grown on minimal medium comprising 5 or 30 g/L sucrose.

The following examples will serve as further illustration and clarification of the present invention and are not intended to be limiting.

### EXAMPLES

### Example 1. Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4·7H2O, 30 g/L sucrose or 30 g/L glycerol, 1 ml/L vitamin solution, 100 μIA molybdate solution, and 1 mL/L selenium solution. As specified in the respective examples, 0.30 g/L sialic acid and/or 20 g/L lactose were additionally added to the medium as precursor(s). The minimal medium was set to a pH of 7 with 1M KOH. Vitamin solution consisted of 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3BO4, 0.4 g/L Na2EDTA·2H2O and 1.01 g/L thiamine·HCl. The molybdate solution contained 0.967 g/L NaMoO4·2H2O. The selenium solution contained 42 g/L Seo2. The minimal medium for fermentations contained 6.75 g/L NH4C1, 1.25 g/L (NH4)2SO4, 2.93 g/L KH2PO4 and 7.31 g/L KH2PO4, 0.5 g/L NaCl, 0.5 g/L MgSO4·7H2O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid and/or 20 g/L lactose were additionally added to the medium as precursor(s).

Complex medium was sterilized by autoclaving (121° C., 21 min) and minimal medium by filtration (0.22 μm Sartorius). When necessary, the medium was made selective by adding an antibiotic: e.g. chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZΔM15, Δ(lacZYA-argF) U169, deoR, recA1, endA1, hsdR17(rk⁻, mk⁺), phoA, supE44, lambda⁻, thi-1, gyrA96, relA1) bought from Invitrogen.

### Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in mL LB media with ampicillin, (100 mg/L) and L-arabinose (10 mM) at 30° C. to an OD₆₀₀nm of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

For GDP-fucose production upon sucrose, the mutant strain was derived from E. coli K12 MG1655 comprising knock-outs of the E. coli wcaJ and thyA genes and genomic knock-ins of constitutive transcriptional units containing a sucrose permease chosen from the list comprising SEQ ID NOs 01 to 62 and 98 to 121, a fructose kinase (Frk) originating from Zymomonas mobilis with SEQ ID NO 63 and a sucrose phosphorylase (SP) originating from Bifidobacterium adolescentis with SEQ ID NO 64. For production of fucosylated oligosaccharides, the mutant GDP-fucose production strain was additionally modified with expression plasmids comprising constitutive transcriptional units for an alpha-1,2-fucosyltransferase chosen from the list comprising SEQ ID NO 65 (H. pylori), SEQ ID NO 66 (Helicobacter sp.), SEQ ID NO 67 (Porphyromonas catoniae ATCC 51270), and SEQ ID NO 68 (Akkermansia muciniphila) and/or an alpha-1,3-fucosyltransferase chosen from the list comprising SEQ ID NO 69 (Basilea psittacipulmonis JF4266) and SEQ ID NO 70 (Azospirillum oryzae A2P) and with a constitutive transcriptional unit for the E. coli thyA with SEQ ID NO 71 as selective marker. The constitutive transcriptional units of the fucosyltransferase genes could also be present in the mutant E. coli strain via genomic knock-ins. GDP-fucose production can further be optimized in the mutant E. coli strain by genomic knock-outs of the E. coli genes comprising glgC, agp, pfkA, pfkB, pgi, arcA, iclR, pgi and Ion as described in WO2016075243 and WO2012007481. GDP-fucose production can additionally be optimized comprising genomic knock-ins of constitutive transcriptional units for the E. coli manA with SEQ ID NO 72, manB with SEQ ID NO 73, manC with SEQ ID NO 74, gmd with SEQ ID NO 75 and fcl with SEQ ID NO 76. GDP-fucose production can also be obtained by genomic knock-outs of the E. coli fucK and fucI genes and genomic knock-ins of constitutive transcriptional units containing the fucose permease (fucP) from E. coli with SEQ ID NO 77 and the bifunctional fucose kinase/fucose-1-phosphate guanylyltransferase (fkp) from Bacteroides fragilis with SEQ NO ID 78. If the mutant strains producing GDP-fucose were intended to make fucosylated lactose structures, the strains were additionally modified with genomic knock-outs of the E. coli LacZ, LacY and LacA genes and with a genomic knock-in of a constitutive transcriptional unit for the E. coli LacY with SEQ ID NO 79.

For sialic acid production upon sucrose, the mutant strain was derived from E. coli K12 MG1655 comprising knock-outs of the E. coli nagA and nagB genes and genomic knock-ins of constitutive transcriptional units containing a sucrose permease chosen from the list comprising SEQ ID NOs 01 to 62 and 98 to 121, a fructose kinase (Frk) originating from Z. mobilis with SEQ ID NO 63, a sucrose phosphorylase (SP) originating from B. adolescentis with SEQ ID NO 64, a glucosamine 6-phosphate N-acetyltransferase (GNA1) from Saccharomyces cerevisiae with SEQ ID NO 80, an N-acetylglucosamine 2-epimerase (AGE) from Bacteroides ovatus with SEQ ID NO 81 and an N-acetylneuraminate (Neu5Ac) synthase (NeuB) from Neisseria meningitidis with SEQ ID NO 82. Sialic acid production can further be optimized in the mutant E. coli strain with genomic knock-outs of the E. coli genes comprising nagC, nagD, nagE, nanA, nanE, nanK, manX, manY and manZ as described in WO18122225 and with genomic knock-ins of constitutive transcriptional units comprising a mutated variant of the L-glutamine-D-fructose-6-phosphate aminotransferase (glmS*54) from E. coli with SEQ ID NO 83 (differing from the wild-type E. coli glmS by an A39T, an R250C and an G472S mutation) and the phosphatase yqaB from E. coli with SEQ ID NO 84. Sialic acid production upon sucrose can also be obtained by knock-outs of the E. coli nagA and nagB genes and genomic knock-ins of constitutive transcriptional units containing a sucrose permease chosen from the list comprising SEQ ID NOs 01 to 62 and 98 to 121, a fructose kinase (Frk) originating from Z. mobilis with SEQ ID NO 63, a sucrose phosphorylase (SP) originating from B. adolescentis with SEQ ID NO 64, the phosphoglucosamine mutase (glmM) from E. coli with SEQ ID NO 96, the N-acetylglucosamine-1-phosphate uridyltransferase/glucosamine-1-phosphate acetyltransferase (gImU) from E. coli with SEQ ID NO 97, the UDP-N-acetylglucosamine 2-epimerase (NeuC) from Campylobacter jejuni with SEQ ID NO 85 and the N-acetylneuraminate synthase (NeuB) from N. meningitidis with SEQ ID NO 82. Also in this mutant strain, sialic acid production can further be optimized with genomic knock-ins of constitutive transcriptional units comprising the mutant glmS*54 from E. coli with SEQ ID NO 83 and the phosphatase yqaB from E. coli with SEQ ID NO 84. For sialylated oligosaccharide production, the sialic acid production strains further need to express an N-acylneuraminate cytidylyltransferase (NeuA) from Pasteurella multocida with SEQ ID NO 86, and a beta-galactoside alpha-2,3-sialyltransferase comprising SEQ ID NO 87 (PmultST3) from P. multocida and/or SEQ ID NO 88 (NmeniST3) from N. meningitidis, and/or a beta-galactoside alpha-2,6-sialyltransferase comprising SEQ ID NO 89 (PdST6) from Photobacterium damselae and/or SEQ ID NO 90 (P-JT-ISH-224-5T6) from Photobacterium sp. JT-ISH-224. Constitutive transcriptional units of PmNeuA and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for the E. coli LacY with SEQ ID NO 79.

To produce LN3 (GlcNAc-b1,3-Gal-b1,4-Glc) and oligosaccharides originating thereof comprising lacto-N-tetraose (LNT) and lacto-N-neotetraose (LNnT) upon sucrose, the mutant strain was derived from E. coli K12 MG1655 and modified with a knock-out of the E. coli LacZ and nagB genes and with genomic knock-ins of constitutive transcriptional units containing a sucrose permease chosen from the list comprising SEQ ID NOs 01 to 62 and 98 to 121, a fructose kinase (Frk) originating from Z. mobilis with SEQ ID NO 63, a sucrose phosphorylase (SP) originating from B. adolescentis with SEQ ID NO 64 and the galactoside beta-1,3-N-acetylglucosaminyltransferase (LgtA) from N. meningitidis with SEQ ID NO 91. For LNT or LNnT production, the mutant strain is further modified with constitutive transcriptional units for the N-acetylglucosamine beta-1,3-galactosyltransferase (WbgO) from E. coli 055:H7 with SEQ ID NO 92 or the N-acetylglucosamine beta-1,4-galactosyltransferase (LgtB) from N. meningitidis with SEQ ID NO 93, respectively, that can be delivered to the strain either via genomic knock-in or from an expression plasmid. Optionally, multiple copies of the LgtA, wbgO and/or LgtB genes could be added to the mutant E. coli strains. Also, LNT and/or LNnT production can be enhanced by improved UDP-GlcNAc production by modification of the strains with one or more genomic knock-ins of a constitutive transcriptional unit for glmS*54 from E. coli with SEQ ID NO 83. In addition, the strains can optionally be modified for enhanced UDP-galactose production with genomic knock-outs of the E. coli ushA and galT genes. The mutant E. coli strains can also optionally be adapted with a genomic knock-in of a constitutive transcriptional unit for the UDP-glucose-4-epimerase (galE) from E. coli with SEQ ID NO 94, the phosphoglucosamine mutase (glmM) from E. coli with SEQ ID NO 96 and the N-acetylglucosamine-1-phosphate uridyltransferase/glucosamine-1-phosphate acetyltransferase (glmU) from E. coli with SEQ ID NO 97.

Preferably but not necessarily, the glycosyltransferases were N-terminally fused to an MBP-tag to enhance their solubility (Fox et al., Protein Sci. 2001, 10(3), 622-630).

All constitutive promoters, UTRs and terminator sequences originated from the libraries described by Mutalik et al. (Nat. Methods 2013, No. 10, 354-360) and Cambray et al. (Nucleic Acids Res. 2013, 41(9), 5139-5148): the genes were expressed using promoters MutalikP5 (“PROM0005_MutalikP5”) and apFAB82 (“PROM0050_apFAB82”) as described by Mutalik et al. (Nat. Methods 2013, No. 10, 354-360), UTRs used comprised GalE_BCD12 (“UTR0010_GalE_BCD12”) and GalE_LeuAB (“UTR0014_GalE_LeuAB”) as described by Mutalik et al. (Nat. Methods 2013, No. 10, 354-360), and terminator sequence used was ilvGEDA (“TER0007_ilvGEDA”) as described by Cambray et al. (Nucleic Acids Res. 2013, 41(9), 5139-5148). All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96well square microtiter plate, with 400 μL minimal medium by diluting 400×. These final 96-well culture plates were then incubated at 37° C. on an orbital shaker at 800 rpm for 72h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2SO4 and 20% NH₄OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Analytical Analysis

Standards such as but not limited to sucrose, lactose, TEL, 3-FL, DiFL, 3′SL, 6′SL, lacto-N-triose II (LN3), lacto-N-tetraose (LNT), lacto-N-neo-tetraose (LNnT), LNEP-1, LNEP-11, LNEP-111, LNFP-V, LNEP-VI, LSTa, LSTc and LSTd were purchased from Carbosynth (UK), Elicityl (France) and IsoSep (Sweden). Other compounds were analysed with in-house made standards.

Neutral oligosaccharides were analysed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and % acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of % acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analysed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Example 2. Evaluation of Growth of an E. coli 3-Fucosyllactose Production Strain Expressing the Wild Type CscB Sucrose Permease from E. coli W when Cultivated in Medium with Decreasing Sucrose Concentrations

An E. coli strain modified for GDP-fucose production as described in Example 1 was adapted for growth on sucrose by genomic knock-ins of constitutive transcriptional units for the wild-type sucrose permease CscB with SEQ ID NO 01 from E. coli W, the fructose kinase (Frk) from Zymomonas mobilis with SEQ ID NO 63 and the sucrose phosphorylase (SP) from Bifidobacterium adolescentis with SEQ ID NO 64. In a next step, the strain was further transformed with an expression plasmid having a constitutive transcriptional unit to express the a1,3-fucosyltransferase with SEQ ID NO 69 from Basilea psittacipulmonis. The novel strain was evaluated in a growth experiment according to the culture conditions provided in Example 1, in which the culture medium comprised 30, 8 or 4 g/L sucrose. The strain was grown in three biological replicates in a 96-well plate. Based on the absorbance measurements at 600 nm, the maximal growth rate (μMax) was calculated and normalized to the average μMax observed when grown in medium comprising g/L of sucrose. FIG. 1 demonstrates that the normalized maximal growth rate (μMax in %) of the strain decreases with decreasing sucrose concentration in the medium. Such a strain thus grows slower if lower amounts of sucrose are available in the medium.

### Example 3. Evaluation of Growth of E. coli Strains Expressing Various Sucrose Permeases when Cultivated in Medium with Decreasing Sucrose Concentrations

An E. coli strain modified for GDP-fucose production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant thereof containing a V316A, C327G and/or F371V mutation as specified in SEQ ID NOs 33, 35, 36, 37, 38, 39, 40. In a next step, the strains were further transformed with an expression plasmid having a constitutive transcriptional unit to express either an a1,2-fucosyltransferase with SEQ ID NO 65 from H. pylori or an a1,3-fucosyltransferase selected from B. psittacipulmonis (SEQ ID NO 69) or from A. oryzae (SEQ ID NO 70). The novel strains were evaluated in a growth experiment according to the culture conditions provided in Example 1, in which the culture medium comprised 30, 8 or 4 g/L sucrose. The strains were grown in three biological replicates in a 96-well plate. Based on the absorbance measurements at 600 nm, the maximal growth rate (μMax) was calculated and normalized to the average μMax observed for the reference strain having the wild-type CscB gene from E. coli W with SEQ ID NO 01 when grown in medium comprising 30 g/L of sucrose. FIGS. 2, 3 and 4 show that in conditions where the sucrose concentration in the medium was low (in this experiment 4 or 8 g/L), the normalized maximal growth rate (μMax in %) of the strains expressing a sucrose permease with SEQ ID NO 33, 35, 36, 37, 38, 39 or 40 was higher than the normalized maximal growth rate of the respective reference strain expressing the wild type CscB from E. coli W with SEQ ID NO 01. This observation was independent of the fucosyltransferase expressed. Additionally, in conditions where the sucrose concentration in the medium was high (30 g/L), the normalized maximal growth rate of the strains expressing the sucrose permease with SEQ ID NO 40, differing by a C327G and a F371V mutation compared to the wild-type CscB from E. coli W with SEQ ID NO 01, was higher than the normalized maximal growth rate of the respective reference strain expressing said wild-type CscB with SEQ ID NO 01.

### Example 4. Evaluation of Growth of E. coli 3-FL Production Strains Expressing Various Sucrose Permeases when Cultivated in Medium with Decreasing Sucrose Concentrations

An E. coli strain modified for GDP-fucose production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant thereof differing by a single mutation at position 316 (V316c), position 327 (C327d) or position 371 (F371e) wherein c, d and e were replaced by any amino acid residue possible as specified in SEQ ID NOs 04, 05 and 06, respectively. In a next step, the strains were further transformed with an expression plasmid having a constitutive transcriptional unit to express the a1,3-fucosyltransferase from B. psittacipulmonis with SEQ ID NO 69. The novel strains were evaluated in a growth experiment according to the culture conditions provided in Example 1, in which the culture medium comprised 30, 8 or 4 g/L sucrose. The strains were grown in three biological replicates in a 96-well plate. Based on the absorbance measurements at 600 nm, the maximal growth rate (μMax) was calculated and normalized to the average μMax observed for the reference strain having the wild-type CscB gene from E. coli W with SEQ ID NO 01.

FIG. 5 shows that the normalized maximal growth rate (μMax in %) of the strains expressing a sucrose permease CscB differing from SEQ ID NO 01 by a C327A (SEQ ID NO 100), F371E (SEQ ID NO 102), F371N (SEQ ID NO 121), F371M (SEQ ID NO 120), F371G (SEQ ID NO 119), V316F (SEQ ID NO 99) or V316C (SEQ ID NO 98) mutation was comparable to the normalized growth rate of the reference strain expressing SEQ ID NO 01 on all media tested. The normalized maximal growth rate (μMax in %) of the strains expressing a sucrose permease CscB differing from SEQ ID NO 01 by a C327G (SEQ ID NO 36), F371A (SEQ ID NO 105), F371C (SEQ ID NO 107), F371Q (SEQ ID NO 103), F371L (SEQ ID NO 104), F371T (SEQ ID NO 106), F371S (SEQ ID NO 108), F3711 (SEQ ID NO 109), F371V (SEQ ID NO 37) or V316A (SEQ ID NO 35) mutation was significantly increased compared to the normalized growth rate of the reference strain expressing SEQ ID NO 01 when grown on medium comprising 4 or 8 g/L sucrose.

### Example 5. Evaluation of Growth of E. coli 3-FL Production Strains Expressing Various Sucrose Permeases when Cultivated in Medium with a Low Sucrose Concentration

An E. coli strain modified for GDP-fucose production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant thereof chosen from the list comprising SEQ ID NOs 110, 111, 112, 113 and 114, and having three single point mutations at three positions (V316, C327 and F371) compared to SEQ ID NO 01. In a next step, the strains were further transformed with an expression plasmid having a constitutive transcriptional unit to express the a1,3-fucosyltransferase from B. psittacipulmonis with SEQ ID NO 69. The novel strains were evaluated in a growth experiment according to the culture conditions provided in Example 1, in which the culture medium comprised 30 or 5 g/L sucrose. The strains were grown in three biological replicates in a 96-well plate. Based on the absorbance measurements at 600 nm, the maximal growth rate (μMax) was calculated and normalized to the average μMax observed for the reference strain having the wild-type CscB gene from E. coli W with SEQ ID NO 01.

FIG. 6 shows that the normalized maximal growth rate (μMax in %) of all novel strains expressing a sucrose permease CscB differing from SEQ ID NO 01 by three single point mutations at positions V316, C327 and F371 was significantly increased compared to the normalized maximal growth rate of the reference strain expressing SEQ ID NO 01, when grown on medium comprising 5 g/L sucrose. The normalized maximal growth rate of the strains expressing a sucrose permease with SEQ ID NO 111 (differing from SEQ ID NO 01 by a V316M, C327G and F371G mutation), SEQ ID NO 112 (differing from SEQ ID NO 01 by a V316L, C327G and F371S mutation), SEQ ID NO 113 (differing from SEQ ID NO 01 by a V316F, C327G and F371V mutation) or SEQ ID NO 114 (differing from SEQ ID NO 01 by a V3161, C327G and F371C mutation) was even twice as high as compared to the normalized maximal growth rate of the reference strain expressing SEQ ID NO 01, when grown on medium comprising 5 g/L sucrose.

### Example 6. Evaluation of Growth of E. coli 3-FL Production Strains Expressing Variant Sucrose Permeases when Cultivated in Medium with a Low Sucrose Concentration

An E. coli strain modified for GDP-fucose production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant thereof chosen of the list comprising SEQ ID NOs 40, 115, 116, 117 and 118. Herein, SEQ ID NOs 115 and 116 were different from SEQ ID NO 01 by a C-terminal truncation of 12 or 7 amino acids, respectively. These truncated variants were made by insertion of an in-frame stop codon directly after glutamate 403 (E403*) or glutamate 408 (E408*), respectively. SEQ ID NO 40 differed from SEQ ID NO 01 by a C327G and a F371V mutation. SEQ ID NOs 117 and 118 were different from SEQ ID NO 01 by a C327G and a F371V mutation and an additional C-terminal truncation of 12 or 7 amino acids, respectively. In a next step, the strains were further transformed with an expression plasmid having a constitutive transcriptional unit to express the a1,3-fucosyltransferase from B. psittacipulmonis with SEQ ID NO 69. The novel strains were evaluated in a growth experiment according to the culture conditions provided in Example 1, in which the culture medium comprised 30 or 5 g/L sucrose. The strains were grown in three biological replicates in a 96-well plate. Based on the absorbance measurements at 600 nm, the maximal growth rate (μMax) was calculated and normalized to the average μMax observed for the reference strain having the wild-type CscB gene from E. coli W with SEQ ID NO 01.

FIG. 7 shows that the normalized maximal growth rate (μMax in %) of all novel strains expressing a variant sucrose permease CscB with SEQ ID NO 115, 116, 40, 117 or 118 was significantly increased compared to the normalized maximal growth rate of the reference strain expressing SEQ ID NO 01, when grown on medium comprising 5 g/L sucrose. Thus, a strain expressing a variant sucrose permease with a C-terminal truncation of 12 or 7 amino acid residues compared to the wild-type polypeptide sequence of CscB from E. coli W with SEQ ID NO 01 grew faster than a strain expressing the natural sucrose permease with SEQ ID NO 01 on medium with low (here 5 g/L) sucrose concentrations.

### Example 7. Evaluation of Growth of E. coli 3-FL Production Strains Expressing Variant Sucrose Permeases Expressed from the Genome or from a Plasmid

An E. coli strain modified for GDP-fucose production as described in Example 1 was further adapted by genomic knock-ins of constitutive transcriptional units for the fructose kinase (Frk) from Z. mobilis with SEQ ID NO 63 and the sucrose phosphorylase (SP) from B. adolescentis with SEQ ID NO 64. Next, the strain was modified to contain either (1) a genomic knock-in of a constitutive transcriptional unit for the wild-type CscB sucrose permease from E. coli W with SEQ ID NO 01 or for the variant sucrose permease with SEQ ID NO 40 or (2) an expression plasmid with a constitutive transcriptional unit for the wild-type CscB sucrose permease from E. coli W with SEQ ID NO 01 or for a variant sucrose permease with SEQ ID NO 36 or SEQ ID NO 40. In a final step, the novel strains were further transformed with an expression plasmid having a constitutive transcriptional unit to express the a1,3-fucosyltransferase from B. psittacipulmonis with SEQ ID NO 69. The novel strains were evaluated in a growth experiment according to the culture conditions provided in Example 1, in which the culture medium comprised 30 or 5 g/L sucrose. The strains were grown in three biological replicates in a 96-well plate. Based on the absorbance measurements at 600 nm, the maximal growth rate (μMax) was calculated and normalized to the average μMax observed for the reference strain having the wild-type CscB gene from E. coli W with SEQ ID NO 01.

FIG. 8 shows that the normalized maximal growth rate (μMax in %) of the novel strain expressing the wild-type CscB sucrose permease with SEQ ID NO 01 from plasmid was higher compared to the normalized maximal growth rate of the reference strain expressing SEQ ID NO 01 from its genome, when grown on medium comprising 5 g/L sucrose. FIG. 10 also shows that the normalized maximal growth rate of the strains expressing a variant sucrose permease (SEQ ID NO 36 or 40) from the genome of from a plasmid was increased compared to the normalized maximal growth rate of the reference strain expressing SEQ ID NO 01 from its genome, when grown on medium comprising 5 g/L sucrose. As is shown with the strain expressing the variant sucrose permease with SEQ ID NO 40, the effect caused by the mutated CscB variant in the novel strains (i.e. increased growth rate of the strains on low concentrations of sucrose) is independent from the location of the expression module for the sucrose permease variant.

### Example 8. Evaluation of 2′-Fucosyllactose Production in a Fermentation Process Using E. coli Strains Expressing a Sucrose Permease

An E. coli strain modified for GDP-fucose production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant sucrose permease with SEQ ID NO 36 differing from SEQ ID NO 01 by a C327G mutation. In a next step, the novel strains were further transformed with an expression plasmid having a constitutive transcriptional unit to express the a1,2-fucosyltransferase from Helicobacter sp. MIT 01-6242 with SEQ ID NO 66.

Both novel strains were evaluated in a fed-batch fermentation process as described in Example 1 wherein the lactose concentration in the culture medium was set at 170 g/L and the sucrose concentration at 60 g/L. At the end of the batch, when all sucrose was consumed, a sucrose-limited feed was applied. The same process was performed twice with both strains. At the end of the fermentation, significant amounts of 2′FL were produced with both strains, but the yield of 2′FL on sucrose (gram TEL produced per gram sucrose consumed) was 80% higher for the strain expressing the sucrose permease variant with SEQ ID NO 36 compared to wild-type sucrose permease from E. coli W with SEQ ID NO 01.

### Example 9. Evaluation of Growth of E. coli 2′FL Production Strains Expressing Various Sucrose Permeases

An E. coli strain modified for GDP-fucose production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121. In a next step, the strains were further transformed with an expression plasmid having a constitutive transcriptional unit to express an a1,2-fucosyltransferase with either SEQ ID NO 65 from H. pylori UA1234, SEQ ID NO 66 from Helicobacter sp. MIT 01-6242, SEQ ID NO 67 from P. catoniae or SEQ ID NO 68 from A. muciniphila. When evaluated in a growth experiment according to the culture conditions provided in Example 1 in which the culture medium comprises 5 g/L sucrose, the maximal growth rate of the novel strains expressing a variant sucrose permease is higher compared to the growth rate of a reference strain, expressing wild-type CscB gene from E. coli W with SEQ ID NO 01.

### Example 10. Evaluation of 3-Fucosyllactose (3-FL) Production in a Fermentation Process Using E. coli Strains Expressing a Sucrose Permease

An E. coli strain modified for GDP-fucose production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121. In a next step, the novel strains were further transformed with an expression plasmid having a constitutive transcriptional unit to express an alpha-1,3-fucosyltransferase with either SEQ ID NO 69 from B. psittacipulmonis JF4266 or SEQ ID NO 70 from A. oryzae A2P. When evaluated in a fed-batch fermentation process as described in Example 1 wherein the lactose concentration in the culture medium is set at 170 g/L and the sucrose concentration at 60 g/L and wherein a sucrose-limited feed is applied at the end of the batch, the novel strains expressing a variant sucrose permease produce 3-FL whereby the yield of 3-FL on sucrose (gram 3-FL produced per gram sucrose consumed) is higher compared to the yield of 3-FL on sucrose obtained by the reference strain expressing wild-type CscB gene from E. coli W with SEQ ID NO 01.

### Example 11. Evaluation of Growth of E. coli LNT and LNnT Production Strains Expressing Various Sucrose Permeases

An E. coli strain modified for LN3 production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121. In a next step, the strains were further transformed with an expression plasmid having a constitutive transcriptional unit to express the N-acetylglucosamine beta-1,3-galactosyltransferase (WbgO) from E. coli 055:H7 with SEQ ID NO 92 for LNT production or the N-acetylglucosamine beta-1,4-galactosyltransferase (LgtB) from N. meningitidis with SEQ ID NO 93 for LNnT production. When evaluated in a growth experiment according to the culture conditions provided in Example 1 in which the culture medium comprises 5 g/L sucrose, the maximal growth rate of the novel strains expressing a variant sucrose permease is higher compared to the growth rate of a reference strain, expressing wild-type CscB gene from E. coli W with SEQ ID NO 01.

### Example 12. Evaluation of Growth of E. coli 3′SL and 6′SL Production Strains Expressing Various Sucrose Permeases

An E. coli strain modified for sialic acid production as described in Example 1 was adapted for growth on sucrose by genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121. In a next step, the strains were further transformed with an expression plasmid having a constitutive transcriptional unit to express the N-acylneuraminate cytidylyltransferase (NeuA) from P. multocida with SEQ ID NO 86, combined with a beta-galactoside alpha-2,3-sialyltransferase with either SEQ ID NO 87 from P. multocida or with SEQ ID NO 88 from N. meningitidis to produce 3′SL, or combined with a beta-galactoside alpha-2,6-sialyltransferase with either SEQ ID NO 89 from P. damselae or with SEQ ID NO 90 from Photobacterium sp. JT-ISH-224 to produce 6′SL. When evaluated in a growth experiment according to the culture conditions provided in Example 1 in which the culture medium comprises 5 g/L sucrose, the maximal growth rate of the novel strains expressing a variant sucrose permease is higher compared to the growth rate of a reference strain, expressing wild-type CscB gene from E. coli W with SEQ ID NO 01.

### Example 13. Materials and Methods Bacillus subtilis

### Media

Two media are used to cultivate B. subtilis: i.e. a rich Luria Broth (LB) and a minimal medium for shake flask cultures. The LB medium consisted of 1% tryptone peptone (Difco), 0.5% yeast extract (Difco) and sodium chloride (VWR). Luria Broth agar (LBA) plates consisted of the LB media, with 12 g/L agar (Difco) added. The minimal medium contained 2.00 g/L (NH4)2SO4, 7.5 g/L KH2PO4, 17.5 g/L K2HPO4, 1.25 g/L Na-citrate, 0.25 g/L MgSO4·7H2O, 0.05 g/L tryptophan, from 10 up to 30 g/L sucrose, 10 mL/L trace element mix and 10 mL/L Fe-citrate solution. The medium was set to a pH of 7 with 1 M KOH. Depending on the experiment lactose is added as a precursor. The trace element mix consisted of 0.735 g/L CaCl2·2H2O, 0.1 g/L MnCl2·2H2O, 0.033 g/L CuCl2·2H2O, 0.06 g/L CoCl2·6H2O, 0.17 g/L ZnCl2, 0.0311 g/L H3BO4, 0.4 g/L Na2EDTA·2H2O and 0.06 g/L Na2MoO4. The Fe-citrate solution contained 0.135 g/L FeCl3·6H2O, 1 g/L Na-citrate (Hoch 1973 PMC1212887).

Complex medium, e.g. LB, was sterilized by autoclaving (121° C., 21′) and minimal medium by filtration (0.22 μm Sartorius). When necessary, the medium was made selective by adding an antibiotic (e.g. zeocin (20 mg/L)).

### Strains, Plasmids and Mutations

Bacillus subtilis 168 is used as available at the Bacillus Genetic Stock Center (Ohio, USA).

Plasmids for gene deletion via Cre/lox are constructed as described by Yan et al. (Appl & Environm microbial, September 2008, p 5556-5562). Gene disruption is done via homologous recombination with linear DNA and transformation via the electroporation as described by Xue et al. (J. microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

For GDP-fucose production, the mutant strain was derived from B. subtilis comprising knockouts of the B. subtilis thyA gene and genomic knock-ins of constitutive transcriptional units containing a sucrose permease chosen from the list comprising SEQ ID NOs 01 to 62 and 98 to 121, a fructose kinase (Frk) originating from Zymomonas mobilis with SEQ ID NO 63 and a sucrose phosphorylase (SP) originating from Bifidobacterium adolescentis with SEQ ID NO 64, the E. coli genes manA with SEQ ID NO 72, manB with SEQ ID NO 73, manCwith SEQ ID NO 74, gmd with SEQ ID NO 75 and fcl with SEQ ID NO 76. For production of fucosylated oligosaccharides, the mutant GDP-fucose production strain was additionally modified with expression plasmids comprising constitutive transcriptional units for an alpha-1,2-fucosyltransferase chosen from the list comprising SEQ ID NO 65 (H. pylori), SEQ ID NO 66 (Helicobacter sp.), SEQ ID NO 67 (Porphyromonas catoniae ATCC 51270), and SEQ ID NO 68 (Akkermansia muciniphila) and/or an alpha-1,3-fucosyltransferase chosen from the list comprising SEQ ID NO 69 (Basilea psittacipulmonis JF4266) and SEQ ID NO 70 (Azospirillum oryzae A2P) and with a constitutive transcriptional unit for the E. coli thyA with SEQ ID NO 71 as selective marker.

For LN3 production, the mutant strain was derived from B. subtilis comprising knockouts of the B. subtilis nagB, glmS and gamA genes and genomic knock-ins of constitutive transcriptional units containing a sucrose permease chosen from the list comprising SEQ ID NOs 01 to 62 and 98 to 121, a fructose kinase (Frk) originating from Zymomonas mobilis with SEQ ID NO 63 and a sucrose phosphorylase (SP) originating from Bifidobacterium adolescentis with SEQ ID NO 64, the mutant glmS*54 from E. coli with SEQ ID NO 83, a galactoside beta-1,3-N-acetylglucosaminyltransferase (IgtA) with SEQ ID NO 91 from N. meningitidis and a lactose permease (LacY) with SEQ ID NO 79 from E. coli. For LNT or LNnT production, the LN3 producing strain was further transformed with constitutive transcriptional units for either an N-acetylglucosamine beta-1,3-galactosyltransferase (wbgO) with SEQ ID NO 92 from E. coli 055:H7 or an N-acetylglucosamine beta-1,4-galactosyltransferase (IgtB) with SEQ ID NO 93 from N. meningitidis, respectively.

For sialic acid (Neu5Ac) production, the mutant strain was derived from B. subtilis comprising knockouts of the B. subtilis nagA, nagB, glmS and gamA genes and genomic knock-ins of constitutive transcriptional units containing a sucrose permease chosen from the list comprising SEQ ID NOs 01 to 62 and 98 to 121, a fructose kinase (Frk) originating from Zymomonas mobilis with SEQ ID NO 63 and a sucrose phosphorylase (SP) originating from Bifidobacterium adolescends with SEQ ID NO 64, the mutant glmS*54 from E. coli with SEQ ID NO 83, a glucosamine 6-phosphate N-acetyltransferase (GNA1) from S. cerevisiae with SEQ ID NO 80, the phosphatase YqaB from E. coli with SEQ ID NO 84, an N-acetylglucosamine 2-epimerase (AGE) from B. ovatus with SEQ ID NO 81 and an N-acetylneuraminate (Neu5Ac) synthase (neuB) from N. meningitidis with SEQ ID NO 82. For sialylated oligosaccharide production, the sialic acid production strains further need to express an N-acylneuraminate cytidylyltransferase (neuA) from P. multocida with SEQ ID NO 86, and a beta-galactoside alpha-2,3-sialyltransferase comprising SEQ ID NO 87 from P. multocida and/or SEQ ID NO 88 from N. meningitidis, and/or a beta-galactoside alpha-2,6-sialyltransferase comprising SEQ ID NO 89 from P. damselae and/or SEQ ID NO 90 from Photobacterium sp. JT-ISH-224. Constitutive transcriptional units of neuA and the sialyltransferases can be delivered to the mutant strain either via genomic knock-in or via expression plasmids. If the mutant strains producing sialic acid and CMP-sialic acid were intended to make sialylated lactose structures, the strains were additionally modified with a genomic knock-in of a constitutive transcriptional unit for the E. coli LacY with SEQ ID NO 79.

### Heterologous and Homologous Expression

Genes that needed to be expressed, be it from a plasmid or from the genome were synthetically synthetized with one of the following companies: DNA2.0, Gen9, Twist Biosciences or IDT. Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72h, or shorter, or longer. At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Optical Density, pH and Analytical Analysis

The determination of the optical density and the pH of the bacterial cultures as well as the analytical analysis were performed as described in Example 1.

### Example 14. Evaluation of B. subtilis 3-FL Production Strains Expressing Various Sucrose Permeases

A B. subtilis strain was first modified by a genomic knock-out of the thyA gene and genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121, the E. coli genes LacY with SEQ ID NO 79, manA with SEQ ID NO 72, manB with SEQ ID NO 73, manC with SEQ ID NO 74, gmd with SEQ ID NO 75 and fcl with SEQ ID NO 76. In a next step, the mutant strains were transformed with an expression plasmid containing constitutive transcriptional units for the alpha-1,3-fucosyltransferase from B. psittacipulmonis JF4266 with SEQ ID NO 69 and the E. coli thyA gene with SEQ ID NO 71 as selective marker. When evaluated in a growth experiment according to the culture conditions provided in Example 13 in which the culture medium comprises sucrose and lactose, the novel strains produce 3-FL.

### Example 15. Evaluation of B. subtilis LNT and LNnT Production Strains Expressing Various Sucrose Permeases

A B. subtilis strain was first modified by a genomic knock-out of the nagB, glmS, gamA and thyA genes and genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121, the mutant glmS*54 from E. coli with SEQ ID NO 83, a galactoside beta-1,3-N-acetylglucosaminyltransferase (IgtA) with SEQ ID NO 91 from N. meningitidis and a lactose permease (LacY) with SEQ ID NO 79 from E. coli. In a next step, the mutant strains were further transformed with an expression plasmid containing constitutive transcriptional units for E. coli thyA with SEQ ID NO 71 as selective marker and either an N-acetylglucosamine beta-1,3-galactosyltransferase (wbgO) with SEQ ID NO 92 from E. coli 055:H7 to produce LNT or an N-acetylglucosamine beta-1,4-galactosyltransferase (IgtB) with SEQ ID NO 93 from N. meningitidis to produce LNnT. When evaluated in a growth experiment according to the culture conditions provided in Example 13 in which the culture medium comprises sucrose and lactose, the novel strains expressing the N-acetylglucosamine beta-1,3-galactosyltransferase produce LN3 and LNT whereas the novel strains expressing the N-acetylglucosamine beta-1,4-galactosyltransferase produce LN3 and LNnT.

### Example 16. Evaluation of B. subtilis 3′SL Production Strains Expressing Various Sucrose Permeases

A B. subtilis strain was first modified by a genomic knock-out of the nagA, nagB, glmS, gamA and thyA genes and genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121, the mutant glmS*54 from E. coli with SEQ ID NO 83, a glucosamine 6-phosphate N-acetyltransferase (GNA1) from S. cerevisiae with SEQ ID NO 80, the phosphatase YqaB from E. coli with SEQ ID NO 84, an N-acetylglucosamine 2-epimerase (AGE) from B. ovatus with SEQ ID NO 81, an N-acetylneuraminate (Neu5Ac) synthase (neuB) from N. meningitidis with SEQ ID NO 82 and the E. coli LacY with SEQ ID NO 79. In a next step, the mutant strains were further transformed with an expression plasmid containing constitutive transcriptional units for the E. coli thyA with SEQ ID NO 71 as selective marker, an N-acylneuraminate cytidylyltransferase (neuA) from P. multocida with SEQ ID NO 86, and a beta-galactoside alpha-2,3-sialyltransferase comprising SEQ ID NO 87 from P. multocida. When evaluated in a growth experiment according to the culture conditions provided in Example 13 in which the culture medium comprises sucrose and lactose, the novel strains produce 3′SL.

### Example 17. Materials and Methods Corynebacterium glutamicum

### Media

Two different media are used to cultivate C. glutamicum: i.e. a rich tryptone-yeast extract (TY) medium and a minimal medium. The TY medium consisted of 1.6% tryptone (Difco), 1% yeast extract (Difco) and 0.5% sodium chloride (VWR). TY agar (TYA) plates consisted of the TY media, with 12 g/L agar (Difco) added. The minimal medium for the shake flask experiments contained 20 g/L (NH4)2SO4, 5 g/L urea, 1 g/L KH2PO4, 1 g/L K2HPO4, 0.25 g/L MgSO4·7H2O, 42 g/L MOPS, from 10 up to 30 g/L sucrose (or another carbon source including but not limited to glucose, fructose, maltose, glycerol and maltotriose) and 1 mL/L trace element mix. Depending on the experiment lactose is added as a precursor. The trace element mix consisted of 10 g/L CaCl₂), 10 g/L FeSO4·7H2O, 10 g/L MnSO4·H2O, 1 g/L ZnSO4·7H2O, 0.2 g/L CuSO4, 0.02 g/L NiCl2·6H2O, 0.2 g/L biotin (pH 7.0) and 0.03 g/L protocatechuic acid.

Complex medium, e.g. TY, was sterilized by autoclaving (121° C., 21′) and minimal medium by filtration (0.22 μm Sartorius). When necessary, the medium was made selective by adding an antibiotic (e.g. kanamycin, ampicillin).

### Strains and Mutations

Corynebacterium glutamicum ATCC 13032 was used as available at the American Type Culture Collection. Integrative plasmid vectors based on the Cre/loxP technique as described by Suzuki et al. (Appl. Microbiol. Biotechnol., 2005 April, 67(2):225-33) and temperature-sensitive shuttle vectors as described by Okibe et al. (J. Microbiol. Meth. 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

For Neu5Ac production, the mutant strain was derived from C. glutamicum comprising knockouts of the C. glutamicum Idh, cgl2645, nagB, glmS and nanA genes and genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121, mutant glmS*54 from E. coli with SEQ ID NO 83, a glucosamine 6-phosphate N-acetyltransferase (GNA1) from S. cerevisiae with SEQ ID NO 80, an N-acetylglucosamine 2-epimerase (AGE) from B. ovatus with SEQ ID NO 81 and an N-acetylneuraminate (Neu5Ac) synthase (neuB) from N. meningitidis with SEQ ID NO 82. For sialylated oligosaccharide production, the sialic acid production strains further need to express an N-acylneuraminate cytidylyltransferase (neuA) from P. multocida with SEQ ID NO 86, and a beta-galactoside alpha-2,3-sialyltransferase comprising SEQ ID NO 87 from P. multocida and/or SEQ ID NO 88 from N. meningitidis, and/or a beta-galactoside alpha-2,6-sialyltransferase comprising SEQ ID NO 89 from P. damselae and/or SEQ ID NO 90 from Photobacterium sp. JT-ISH-224. Constitutive transcriptional units of neuA and the sialyltransferases can be delivered to the mutant strain either via genomic knock-in or via expression plasmids. If the mutant strains producing sialic acid and CMP-sialic acid were intended to make sialylated lactose structures, the strains were additionally modified with a genomic knock-in of a constitutive transcriptional unit for the E. coli LacY with SEQ ID NO 79.

For LN3 production, the mutant strain was derived from C. glutamicum comprising knockouts of the C. glutamicum Idh, cg12645, nagB and glmS genes and genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121, mutant glmS*54 from E. coli with SEQ ID NO 83, a galactoside beta-1,3-N-acetylglucosaminyltransferase (IgtA) with SEQ ID NO 91 from N. meningitidis and a lactose permease (LacY) with SEQ ID NO 79 from E. coli. For LNT or LNnT production, the LNT3 producing strain was further transformed with constitutive transcriptional units for either an N-acetylglucosamine beta-1,3-galactosyltransferase (wbgO) with SEQ ID NO 92 from E. coli 055:H7 or an N-acetylglucosamine beta-1,4-galactosyltransferase (IgtB) with SEQ ID NO 93 from N. meningitidis, respectively.

### Heterologous and Homologous Expression

Genes that needed to be expressed, be it from a plasmid or from the genome were synthetically synthetized with one of the following companies: Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL minimal medium by diluting 400×. Each strain was grown in multiple wells of the 96-well plate as biological replicates. These final 96-well culture plates were then incubated at 37° C. on an orbital shaker at 800 rpm for 72h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index was determined by dividing the oligosaccharide concentrations, e.g. sialyllactose concentrations, measured in the whole broth by the biomass, in relative percentages compared to the reference strain. The biomass is empirically determined to be approximately ⅓rd of the optical density measured at 600 nm.

### Optical Density, pH and Analytical Analysis

The determination of the optical density and the pH of the bacterial cultures as well as the analytical analysis were performed as described in Example 1.

### Example 18. Evaluation of C. glutamicum 3′SL or 6′SL Production Strains Expressing Various Sucrose Permeases

A wild-type C. glutamicum strain is first modified with genomic knockouts of the C. glutamicum genes ldh, cg12645, nagB, glmS and nanA, together with genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121, the mutant glmS*54 from E. coli with SEQ ID NO 83, GNA1 with SEQ ID NO 80 from S. cerevisiae, AGE with SEQ ID NO 81 from B. ovatus, neuB with SEQ ID NO 82 from N. meningitidis and LacY with SEQ ID NO 79 from E. coli. In a next step, the novel strain is transformed with an expression plasmid comprising constitutive transcriptional units for neuA with SEQ ID NO 86 from P. multocida and either an alpha-2,3-sialyltransferase (like SEQ ID NO 87 from P. multocida or SEQ ID NO 88 from N. meningitidis) or an alpha-2,6-sialyltransferase (like SEQ ID NO 89 from P. damselae or SEQ ID NO 90 from Photobacterium sp. JT-ISH-224). When evaluated in a 3-days growth experiment according to the culture conditions provided in Example 17 using appropriate selective medium comprising sucrose and lactose, the novel strains expressing an alpha-2,3-sialyltransferase synthesize 3′SL whereas the novel strains expressing an alpha-2,6-sialyltransferase synthesize 6′SL.

### Example 19. Evaluation of C. glutamicum LNT or LNnT Production Strains Expressing Various Sucrose Permeases

A wild-type C. glutamicum strain is first modified with genomic knockouts of the C. glutamicum genes ldh, cgl2645, nagB and glmS together with genomic knock-ins of constitutive transcriptional units for the fructose kinase (Frk) from Z. mobilis with SEQ ID NO 63, the sucrose phosphorylase (SP) from B. adolescentis with SEQ ID NO 64 and either the wild-type E. coli W cscB sucrose permease with SEQ ID NO 01 or a variant chosen from the list comprising SEQ ID NOs 01 to 62 and 98 to 121, the mutant glmS*54 from E. coli with SEQ ID NO 83, the lactose permease LacY with SEQ ID NO 79 from E. coli and IgtA from N. meningitidis with SEQ ID NO 91. When evaluated in a 3-days growth experiment according to the culture conditions provided in Example 17 using appropriate selective medium comprising sucrose and lactose, the novel strain synthesizes LN3.

In a next step for LNT or LNnT production, the LN3 producing C. glutamicum strain is further modified with constitutive transcriptional units for either wbgO from E. coli 055:H7 with SEQ ID NO 92 or IgtB from N. meningitidis with SEQ ID NO 93, respectively. When evaluated in a 3-days growth experiment according to the culture conditions provided in Example 17 using appropriate selective medium comprising sucrose and lactose, the novel strains expressing the N-acetylglucosamine beta-1,3-galactosyltransferase wbgO produce LN3 and LNT whereas the novel strains expressing the N-acetylglucosamine beta-1,4-galactosyltransferase IgtB produce LN3 and LNnT.

1.-45. (canceled)

46. A sucrose permease having sucrose permease activity and comprising a polypeptide that has at least 80% overall sequence identity to SEQ ID NO: 1 and that
i) differs from SEQ ID NO: 1 by having a different amino acid for proline at position 169 (P169a), for valine at position 316 (V316c) or for phenylalanine at position 371 (F371e) wherein a, c, and e can be any amino acid residue excluding histidine for residue a, or
ii) shares the serine at position 246 of SEQ ID NO: 1 and differs from SEQ ID NO: 1 by having a different amino acid for tryptophan at position 230 (W230b) or for cysteine at position 327 (C327d), wherein b and d can be any amino acid residue, or
iii) differs from SEQ ID NO: 1 by having at least two amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue, or
iv) differs from SEQ ID NO: 1 by having three amino acid differences selected from the group consisting of proline at position 169 (P169f), tryptophan at position 230 (W230b), valine at position 316 (V316c), cysteine at position 327 (C327d) and phenylalanine at position 371 (F371e), or
v) differs from SEQ ID NO: 1 by having four amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue, or
vi) differs from SEQ ID NO: 1 by having five amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue.

47. The sucrose permease of claim 46, wherein the sucrose permease:
i) differs from SEQ ID NO: 1 by having a different amino acid for proline at position 169 (P169a), for valine at position 316 (V316c) or for phenylalanine at position 371 (F371e) wherein a and e can be any amino acid residue excluding histidine for residue a, and wherein c can be any amino acid residue excluding methionine, or
ii) shares the serine at position 246 of SEQ ID NO: 1 and differs from SEQ ID NO: 1 by having a different amino acid for tryptophan at position 230 (W230b) or for cysteine at position 327 (C327d) wherein b and d can be any amino acid residue, or
iii) differs from SEQ ID NO: 1 by having at least two amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue, or
iv) differs from SEQ ID NO: 1 by having three amino acid differences selected from the group consisting of proline at position 169 (P169f), tryptophan at position 230 (W230b), valine at position 316 (V316c), cysteine at position 327 (C327d) and phenylalanine at position 371 (F371e), or
v) differs from SEQ ID NO: 1 by having four amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue, or
vi) differs from SEQ ID NO: 1 by having five amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue.

48. The sucrose permease of claim 46, wherein the sucrose permease:
i) differs from SEQ ID NO: 1 by having a different amino acid for proline at position 169 (P169a), for valine at position 316 (V316c), or for phenylalanine at position 371 (F371e), wherein a and e can be any amino acid residue excluding histidine for residue a, and wherein c is selected from the group consisting of alanine, cysteine and phenylalanine, or
ii) shares the serine at position 246 of SEQ ID NO: 1 and differs from SEQ ID NO: 1 by having a different amino acid for tryptophan at position 230 (W230b) or for cysteine at position 327 (C327d) wherein b and d can be any amino acid residue, or
iii) differs from SEQ ID NO: 1 by having at least two amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue, or
iv) differs from SEQ ID NO: 1 by having at least three amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue, or
v) differs from SEQ ID NO: 1 by having four amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue, or
vi) differs from SEQ ID NO: 1 by having five amino acid differences selected from the group consisting of proline at position 169 (P169f), tryptophan at position 230 (W230b), valine at position 316 (V316c), cysteine at position 327 (C327d) and phenylalanine at position 371 (F371e), wherein f, b, c, d, and e can be any amino acid residue.

49. The sucrose permease of claim 46, wherein the sucrose permease has an improved affinity for sucrose compared to the sucrose permease of SEQ ID NO: 1.

50. The sucrose permease of claim 46, wherein the sucrose permease
i) comprises the polypeptide of any one of SEQ ID NOs: 26, 5, 16, 2, 3, 4, 6, 7, 8, 9, 10, 11, 12, 13, 14, 15, 17, 18, 19, 20, 21, 22, 23, 24, 25, 27, 28, 29, 30, 31 or 32, or
ii) is a functional homologue, variant or derivative of any one of SEQ ID NOs: 26, 5, 16, 2, 3, 4, 6, 7, 8, 9, 10, 11, 12, 13, 14, 15, 17, 18, 19, 20, 21, 22, 23, 24, 25, 27, 28, 29, 30, 31 or 32, having at least 80% overall sequence identity to the full length of any one of the polypeptides with SEQ ID NOs: 26, 5, 16, 2, 3, 4, 6, 7, 8, 9, 10, 11, 12, 13, 14, 15, 17, 18, 19, 20, 21, 22, 23, 24, 25, 27, 28, 29, 30, 31 or 32 and having sucrose permease activity.

51. The sucrose permease of claim 46, wherein the sucrose permease
i) comprises the polypeptide of SEQ ID NO: 32, or
ii) is a functional homologue, variant or derivative of SEQ ID NO: 32, having at least 80% overall sequence identity to the full length of polypeptides with SEQ ID NO: 32 and having sucrose permease activity.

52. The sucrose permease of claim 46, wherein the sucrose permease
i) comprises the polypeptide of SEQ ID NOs: 27, 28, 29, 30 or 31, or
ii) is a functional homologue, variant or derivative of any one of SEQ ID NOs: 27, 28, 29, 30 or 31, having at least 80% overall sequence identity to the full length of any one of the polypeptides with SEQ ID NOs: 27, 28, 29, 30 or 31 and having sucrose permease activity.

53. The sucrose permease of claim 46, wherein the sucrose permease
i) comprises the polypeptide of SEQ ID NOs: 26, 17, 18, 19, 20, 21, 22, 23, 24 or or
ii) is a functional homologue, variant or derivative of any one of SEQ ID NOs: 26, 17, 18, 19, 20, 21, 22, 23, 24 or 25, having at least 80% overall sequence identity to the full length of any one of the polypeptides with SEQ ID NOs: 26, 17, 18, 19, 20, 21, 22, 23, 24 or 25 and having sucrose permease activity.

54. The sucrose permease of claim 50, wherein the sucrose permease with SEQ ID NO: 26:
differs from SEQ ID NO: 1 by having a different amino acid for valine at position 316, for cysteine at position 327 and for phenylalanine at position 371, or
is a functional homologue, variant or derivative of any one of SEQ ID NOs: 33, 110, 111, 112, 113 or 114, having at least 80% overall sequence identity to the full length of any one of the polypeptides with SEQ ID NOs: 33, 110, 111, 112, 113 or 114 and having sucrose permease activity.

55. The sucrose permease of claim 46, wherein the sucrose permease
i) comprises the polypeptide of SEQ ID NOs: 16, 7, 8, 9, 10, 11, 12, 13, 14 or 15, or
ii) is a functional homologue, variant, or derivative of any one of SEQ ID NOs: 16, 7, 8, 9, 10, 11, 12, 13, 14 or 15, having at least 80% overall sequence identity to the full length of any one of the polypeptides with SEQ ID NOs: 16, 7, 8, 9, 10, 11, 12, 13, 14 or 15 and having sucrose permease activity.

56. The sucrose permease of claim 50, wherein the sucrose permease with SEQ ID NO: 16:
differs from SEQ ID NO: 1 by having a different amino acid for cysteine at position 327 and for phenylalanine at position 371, or
is a functional homologue, variant or derivative of any one of SEQ ID NOs: 40, 117 or 118, having at least 80% overall sequence identity to the full length of any one of the polypeptides with SEQ ID NOs: 40, 117 or 118 and having sucrose permease activity.

57. The sucrose permease of claim 46, wherein the sucrose permease
i) comprises the polypeptide of SEQ ID NOs: 5, 2, 3, 4 or 6, or
ii) is a functional homologue, variant or derivative of any one of SEQ ID NOs: 5, 2, 3, 4 or 6, having at least 80% overall sequence identity to the full length of any one of the polypeptides with SEQ ID NOs: 5, 2, 3, 4 or 6 and having sucrose permease activity.

58. The sucrose permease of claim 50, wherein the sucrose permease with SEQ ID NO: 5:
differs from SEQ ID NO: 1 by having a different amino acid for cysteine at position 327, or
is a functional homologue, variant or derivative of SEQ ID NO: 36 or 100, having at least 80% overall sequence identity to the full length of the polypeptides with SEQ ID NO: 36 or 100 and having sucrose permease activity.

59. The sucrose permease of claim 46, wherein the sucrose permease further differs from SEQ ID NO: 1 by having at least one further amino acid difference selected from the group consisting of:
leucine at position 61 is substituted with proline, tryptophan, histidine, phenylalanine, or tyrosine,
phenylalanine at position 159 is substituted with leucine,
glycine at position 162 is substituted with cysteine,
proline at position 169 is substituted with histidine,
arginine at position 300 is substituted with alanine or leucine,
glutamine at position 353 is substituted with histidine,
truncation of amino acid residues 404 to 415, and
truncation of amino acid residues 409 to 415.

60. The sucrose permease of claim 59, wherein the sucrose permease is:
represented by SEQ ID NO: 118 or 119, or
is a functional homologue, variant or derivative of SEQ ID NO: 118 or 119, having at least 80% overall sequence identity to the full length of the polypeptides with SEQ ID NO: 118 or 119 and having sucrose permease activity.

61. The sucrose permease of claim 46, wherein the sucrose permease comprises of at least 350 amino acids.

62. The sucrose permease of claim 46, wherein the sucrose permease comprises of less than 450 amino acids.

63. A metabolically engineered cell for producing a glycosylated product, the cell comprising a pathway for production of the glycosylated product, wherein the cell is capable of expressing at least one sucrose permease that has sucrose permease activity and comprises a polypeptide that differs from SEQ ID NO: 1 by (i) having at least one amino acid difference selected from the group consisting of: proline at position 169 (P169f), tryptophan at position 230 (W230b), valine at position 316 (V316c), cysteine at position 327 (C327d), and phenylalanine at position 371 (F371e) wherein f, b, c, d, and e can be any amino acid residue and/or (ii) a truncation of amino acid residues 403 to 415, 404 to 415, 405 to 415, 406 to 415, 407 to 415, 408 to 415, 409 to 415, 410 to 415, 411 to 415, 412 to 415, 413 to 415 or 414 to 415.

64. The cell of claim 63, wherein the sucrose permease has an improved affinity for sucrose compared to the sucrose permease of SEQ ID NO: 1.

65. The cell of claim 63, wherein the sucrose permease
i) comprises the polypeptide of any one of SEQ ID NOs: 26, 5, 16, 34, 3, 4, 6, 7, 8, 9, 10, 11, 12, 13, 14, 15, 17, 18, 19, 20, 21, 22, 23, 24, 25, 27, 28, 29, 30, 31, 32, 41, 42, 43, 44, 46, 47, 48, 49, 50, 51, 52, 53, 54, 55, 56, 57, 58, 59, 60, 61, 62, 35, 36, 37, 38, 39, 40, 98, 99, 100, 101, 102, 103, 104, 105, 106, 107, 108, 109, 110, 111, 112, 113, 114, 115, 116, 117, 118, 119, 120, or 121, or
ii) is a functional homologue, variant or derivative of any one of SEQ ID NOs: 26, 5, 16, 34, 3, 4, 6, 7, 8, 9, 10, 11, 12, 13, 14, 15, 17, 18, 19, 20, 21, 22, 23, 24, 25, 27, 28, 29, 30, 31, 32, 41, 42, 43, 44, 45, 46, 47, 48, 49, 50, 51, 52, 53, 54, 55, 56, 57, 58, 59, 60, 61, 62, 35, 36, 37, 38, 39, 40, 98, 99, 100, 101, 102, 103, 104, 105, 106, 107, 108, 109, 110, 111, 112, 113, 114, 115, 116, 117, 118, 119, 120 or 121, having at least 80% overall sequence identity to the full length of any one of the polypeptides with SEQ ID NOs: 26, 5, 16, 34, 3, 4, 6, 7, 8, 9, 10, 11, 12, 13, 14, 15, 17, 18, 19, 20, 21, 22, 23, 24, 25, 27, 28, 29, 30, 31, 32, 41, 42, 43, 44, 45, 46, 47, 48, 49, 50, 51, 52, 53, 54, 55, 56, 57, 58, 59, 60, 61, 62, 35, 36, 37, 38, 39, 40, 98, 99, 100, 101, 102, 103, 104, 105, 106, 107, 108, 109, 110, 111, 112, 113, 114, 115, 116, 117, 118, 119, 120 or 121 and having sucrose permease activity.

66. The cell of claim 63, wherein the sucrose permease further differs from SEQ ID NO: 1 by having at least one further amino acid difference selected from the group consisting of:
leucine at position 61 is substituted with proline, tryptophan, histidine, phenylalanine or tyrosine,
phenylalanine at position 159 is substituted with leucine,
glycine at position 162 is substituted with cysteine,
proline at position 169 is substituted with histidine,
arginine at position 300 is substituted with alanine or leucine,
glutamine at position 353 is substituted with histidine,
truncation of amino acid residues 404 to 415, and
truncation of amino acid residues 409 to 415.

67. The cell of claim 63, wherein the cell is a microorganism, a bacterium, an Escherichia coli strain, an E. coli K-12 strain, or E. coli MG1655.

68. The cell of claim 63, wherein the glycosylated product is selected from the group consisting of di- or oligosaccharides, nucleosides, glycosides and glycolipids, mammalian milk di- or oligosaccharides, and human milk di- or oligosaccharides.

69. A method of producing a glycosylated product by the cell of claim 63, the method comprising:
i) cultivating the cell under conditions permissive to produce the glycosylated product,
ii) optionally, separating the glycosylated product from the cultivation, and
iii) optionally, purifying the glycosylated product from the cell.


SEQUENCE LISTING (121 sequences)

SEQ ID NO: 1 - protein, 415 aa, Escherichia coli W ATCC 9637
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 2 - protein, 415 aa, Artificial sequence
  cscB variant
  UNSURE (169)..(169): Xaa can be any amino acid, any non-classical amino acid residue and any chemical amino acid analogue except His
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 3 - protein, 415 aa, Artificial sequence
  cscB variant
  misc_feature (230)..(230): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 4 - protein, 415 aa, Artificial sequence
  cscB variant
  misc_feature (316)..(316): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 5 - protein, 415 aa, Artificial sequence
  cscB variant
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 6 - protein, 415 aa, Artificial sequence
  cscB variant
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 7 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 8 - protein, 415 aa, Artificial sequence
  cscB variant
  misc_feature (169)..(169): Xaa can be any amino acid, any non-classical amino acid residue and any chemical amino acid analogue
  misc_feature (316)..(316): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 9 - protein, 415 aa, Artificial sequence
  cscB variant
  misc_feature (169)..(169): Xaa can be any amino acid, any non-classical amino acid residue and any chemical amino acid analogue
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 10 - protein, 415 aa, Artificial sequence
  cscB variant
  misc_feature (169)..(169): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 11 - protein, 415 aa, Artificial sequence
  cscB variant
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 12 - protein, 415 aa, Artificial sequence
  cscB variant
  misc_feature (230)..(230): Xaa can be any amino acid, any non-classical amino acid residue and any chemical amino acid analogue
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 13 - protein, 415 aa, Artificial sequence
  cscB variant
  misc_feature (230)..(230): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 14 - protein, 415 aa, Artificial sequence
  cscB variant
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 15 - protein, 415 aa, Artificial sequence
  cscB variant
  misc_feature (316)..(316): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 16 - protein, 415 aa, Artificial sequence
  cscB variant
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 17 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 18 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 19 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 20 - protein, 415 aa, Artificial sequence
  cscB variant
  misc_feature (169)..(169): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 21 - protein, 415 aa, Artificial sequence
  cscB variant
  misc_feature (169)..(169): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 22 - protein, 415 aa, Artificial sequence
  cscB variant
  misc_feature (169)..(169): Xaa can be any amino acid, any non-classical amino acid residue and any chemical amino acid analogue
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 23 - protein, 415 aa, Artificial sequence
  cscB variant
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 24 - protein, 415 aa, Artificial sequence
  cscB variant
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 25 - protein, 415 aa, Artificial sequence
  cscB variant
  misc_feature (230)..(230): Xaa can be any amino acid, any non-classical amino acid residue and any chemical amino acid analogue
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 26 - protein, 415 aa, Artificial sequence
  cscB variant
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 27 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 28 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLCVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 29 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLVKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 30 - protein, 415 aa, Artificial sequence
  cscB variant
  misc_feature (169)..(169): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 31 - protein, 415 aa, Artificial sequence
  cscB variant
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 32 - protein, 415 aa, Artificial sequence
  cscB variant
  misc_feature (169)..(169): Xaa can be any amino acid, any non-classical amino acid residue and any chemical amino acid analogue
  misc_feature (230)..(230): Xaa can be any amino acid, any non-classical amino acid residue and any chemical amino acid analogue
  misc_feature (316)..(316): Xaa can be any amino acid, any non-classical amino acid residue and any chemical amino acid analogue
  misc_feature (327)..(327): Xaa can be any amino acid, any non-classical amino acid residue and any chemical amino acid analogue
  misc_feature (371)..(371): Xaa can be any amino acid, any non-classical amino acid residue and any chemical amino acid analogue
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISXHINFWLVSLFGAVFMMINMRFKDKDHQCVAADAGGVKKEDFIAVFKDRNFWVFVIFIVGTXSFYNIFDQQLFPVFYSGLFESHDVGTRLYGYLNSFQVVLEALCMAIIPFFVNRVGPKNALLIGVVIMALRILSCALFVNPWIISLXKLLHAIEVPLXVISVFKYSVANFDKRLSSTIFLIGFQIASSLGIVLLSTPTGILXDHAGYQTVFFAISGIVCLMLLFGIFFLSKKREQIVMETPVPSAI

SEQ ID NO: 33 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLAKLLHAIEVPLGVISVFKYSVANFDKRLSSTIFLIGFQIASSLGIVLLSTPTGILVDHAGYQTVFFAISGIVCLMLLFGIFFLSKKREQIVMETPVPSAI

SEQ ID NO: 34 - protein, 415 aa, Artificial sequence; identical to SEQ ID NO: 2
  cscB variant
  misc_feature (169)..(169): Xaa can be any amino acid, any non-classical amino acid residue and any chemical amino acid analogue

SEQ ID NO: 35 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLAKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 36 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLGVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 37 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILVDHAGYQTVFFAISGIVCLMLLFGIFFLSKKREQIVMETPVPSAI

SEQ ID NO: 38 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLAKLLHAIEVPLGVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 39 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLAKLLHAIEVPLCVISVFKYSVANFDKRLSSTIFLIGFQIASSLGIVLLSTPTGILVDHAGYQTVFFAISGIVCLMLLFGIFFLSKKREQIVMETPVPSAI

SEQ ID NO: 40 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLGVISVFKYSVANFDKRLSSTIFLIGFQIASSLGIVLLSTPTGILVDHAGYQTVFFAISGIVCLMLLFGIFFLSKKREQIVMETPVPSAI

SEQ ID NO: 41 - protein, 429 aa, Bifidobacterium tsurumiense DSM 17777
MTGSPATSPSRNASMRSSSYFQSSFGVLFFFCALGIWWPFFQRWLTSLGLSGMQIGTVYSLNSLIALIMVLAYGVIQDKLGMKRTLILVVSTIAALIGPFVQFIYAPLITHNLILGTVIGSLVLCAGYISAISLVEAVTERYSRHFGFEYGQSRAWGSLGYAIAVLLAGITFNINPFINFWIASLFGVAMLLVYAVWYTPEQRAERSQESLSDSLPKAPSLREMARVLLMPSLWSMIIFVMLTCTFYTVFDQQFFPSFYTSLFPQESVGNTAYGILNSAHVFAESIMMGTVPVLMRRIGVRKAMLLGACVLCFRITLCALFTDPVIVSAARMLYAIDYPLLALSIFRYFTLHFNPRISATLYMVGFQISAQIGQVIAATPLGIAHDVIGDRATFFIIAGVIVAAIAYGFFVIKRDDQQVDGEPLITVQG

SEQ ID NO: 42 - protein, 425 aa, Actinomyces sp. AS08sgBPME_431
MSQFSQLFKKNSYLYGSATIMLFFMSWGIWWSFFQIWLTSPTGGIQLNGSQVGTVYAINGAGTLILMFLYGAVQDKLYLKRTLVIIVAAIMALIGPFAQFVYQPLLQNNFTLGAVLGGVILSAGFVGAVSLFEAYVERISRVHGFEFGQARMWGSVGYAIVALIAGFMFTINPFVNFWIGSVLGLVLLLIQVFWKTESDETQAAITPDTPTVAEMFALLKDSKVWAVIGFVFLSWTFYTVFDQQMFPDFYTTLFADQATGQQMYGVLNSIQVFLEAAMMALVPLLLNKVGARNSLLLGVTVMFLRIGMCAVFDSAILISAAKLLHAIEVPLFVLGIFKYFSIHFNPALSATLYLVAWNLASQLGNIVLSNPLGRLRDSIGYQPTFAIISGVALVAAAYLLIFLKKDGEAVYKPAESQVISPEPVK

SEQ ID NO: 43 - protein, 422 aa, Corynebacterium sp. NML140438
MEMLRSQFSNPAYRFGSATIFLFFAAWGIWWSFYQIWLTSEAGGLQLNGAQVGTVYAIQGAITMALMLVYGVLQDKLDLKRPLTIFIGASATLIGPFTQFIYHPLLRDHFVVGAILGGVVLSAGFVAGAGLLEAFVERMANRTGFEYGQARMWGSFAYAIVALLAGIFFTINPMLNFWIGSALGALLLCTQLFWHPKGTAQEASIEEVAAKTPPVSEMVGLLKDSHVWKVIVIVLLSWTFYSVFDQQMFPDFYTSLFSSPEIGQRAYGTLNSVQVFLEAIMMGLIPLLMRKVGARNTLLLGIGVMFVRIFLCAAFTGPVLISIAKMLHAFEVPLCILAIFKYFAIHFNKALSATLYLVAFQLSSQLGNVILSTPLGSLRDQIGYQPTFFVISGVVFVAGVIAVFALKRDDEAVVNPEEIPTH

SEQ ID NO: 44 - protein, 422 aa, Corynebacterium sp. HMSC08D02
MEMLRSQFSNPAYRFGSATIFLFFAAWGIWWSFYQIWLTSEAGGLQLNGAQVGTVYAIQGAITMALMLVYGVLQDKLDLKRPLTIFIGASATLIGPFTQFIYHPLLRDHFIVGAILGSVVLSAGFVAGAGLLEAFVERMANRTGFEYGQARMWGSFAYAIVALLAGIFFTINPMLNFWIGSALGALLLCTQLFWHPKGTAQEASIEEVTAKTPPVSEMLGLLKDSHVWKVIVIVLLSWTFYTVFDQQMFPDFYTSLFSSPEVGQRAYGTLNSVQVFLEAIMMGLIPLLMRKVGARNTLLMGIGVMFVRIFMCAAFTGPVLISIAKMLHAFEVPLCILAIFKYFAIHFNKALSATLYLVAFQLSSQLGNVILSTPLGSLRDQIGYQPTFFVISGVVFVAGLIAVFALKRDDEAVVNPEEIPTR

SEQ ID NO: 45 - protein, 422 aa, Corynebacterium sp. MC3
MEMLRSQFSNPAYRFGSATIFLFFAAWGIWWSFYQIWLTSEAGGLQLNGAQVGTVYAIQGAITMALMLVYGVLQDKLDLKRPLTIFIGASATLIGPFTQFIYHPLLRDHFIVGAILGSVVLSAGFVAGAGLLEAFVERMANRTGFEYGQARMWGSFAYAIVALLAGIFFTINPMLNFWIGSALGALLLCTQLFWHPQGTAQEASIEEVTAKTPPISEMLGLLKDSHVWKVIVIVLLSWTFYTVFDQQMFPDFYTSLFSSPEVGQRAYGTLNSVQVFLEAIMMGLIPLLMRKVGARNTLLLGIGVMFVRIFLCAAFGGPVLISIAKMLHAFEVPLCILAIFKYFAIHFNKALSATLYLVAFQLSSQLGNVILSTPLGSLRDQIGYQPTFFVISGVVFLAGVIAVFSLKRDDEAVVNPEEIPTR

SEQ ID NO: 46 - protein, 422 aa, Corynebacterium sp. NML120713
MEMLRSQFSNPAYRFGSATIFLFFAAWGIWWSFYQIWLTSEAGGLQLNGAQVGTVYAIQGAITMALMLVYGVLQDKLDLKRPLTIFIGASATLIGPFTQFIYHPLLRDHFIVGAILGSVVLSAGFVAGAGLLEAFVERMANRTGFEYGQARMWGSFAYAIVALLAGIFFTINPMLNFWIGSALGALLLCTQLFWHPKGTAQEASIEEVTAKTPPVSEMLGLLKDSHVWKVIVIVLLSWTFYTVFDQQMFPDFYTSLFSSPEVGQRAYGTLNSVQVFLEAIMMGLIPLLMRKVGARNTLLLGIGVMFVRIFLCAAFGGPVLISIAKMLHAFEVPLCILAIFKYFAIHFNKALSATLYLVAFQLSSQLGNVILSTPLGSLRDQIGYQPTFFVISGVVFVAGLIAVFALKRDDEAVVNPEEIPTR

SEQ ID NO: 47 - protein, 422 aa, Corynebacterium sp. NML98-0116
MEMLRSQFSNPAYRFGSATIFLFFAAWGIWWSFYQIWLTSEAGGLQLNGAQVGTVYAIQGAITMALMLVYGVLQDKLDLKRPLTIFIGASATLIGPFTQFIYHPLLRDHFIVGAILGSVVLSAGFVAGAGLMEAFVERMANRTGFEYGQARMWGSFAYAIVALLAGIFFTINPMLNFWIGSALGALLLCTQLFWHPKGTAQEASIEEVTAKTPPVSEMLGLLKDSHVWKVIVIVLLSWTFYTVFDQQMFPDFYTSLFSSPEVGQRAYGTLNSVQVFLEAIMMGLIPLLMRKVGARNTLLLGIGVMFVRIFLCAAFGGPVLISIAKMLHAFEVPLCILAIFKYFAIHFNKALSATLYLVAFQLSSQLGNVILSTPLGSLRDQIGYQSTFFVISGVVFVAGVIAVFALKRDDEAVVNPEEIPTR

SEQ ID NO: 48 - protein, 422 aa, Corynebacterium sp. NML130628
MDMLRSQFSNPAYRFGSATIFLFFAAWGIWWSFYQIWLTSEAGGLQLNGAQVGTVYAIQGAITMVLMLVYGVLQDKLDLKRPLTIFIGASATLIGPFTQFIYQPLLQDHFIVGAILGGVVLSAGFVAGAGLLEAFVERMANRTGFEYGQARMWGSFAYAIVALLAGIFFTINPMLNFWIGSALGALLLCTQLFWHPKGTAQEASIEEVTAKTPPVSEMVGLLKDNHVWKVIVIVLLSWTFYSVFDQQMFPDFYTSLFSSPEVGQRAYGTLNSVQVFLEAIMMGLIPLLMRKVGARNTLLLGIGVMFVRIFLCAAFAGPVLISIAKMLHAFEVPLCILAIFKYFAIHFNKALSATLYLVAFQLSSQLGNVILSAPLGSLRDQIGYQPTFFVISGVVFVAGLIAVFALKRDDEAVVNPEEIPTR

SEQ ID NO: 49 - protein, 416 aa, Pluralibacter gergoviae strain 40874
MTLNIPLRNPCYRFASGYSFLFFISWSLWWSLYAIWLKGSLGLSGAALGTLYSVNQFTSMLFMVVYGVVQDRLGLNKRLIWGMGAILLLTGPFIIYLYEPLLKSHFTAGLLLGSVFFGLGYLAGNGLLDSFIEKMSRRWDFEYGTARAWGSFGYAIGALCAGVLFSINPHINFWLVSLFGALFMLINLRFRPDQATARCAEAAKVSRTDFAAVFKDANFWILVLFIIGSWSFYTIYDQQLFPVFYAGLFDSPALGARVYGYLNAFQVVLEALCMAVIPFLINRIGPKNGLLLSVAIMMLRIFSCAVFNDPWIISLVKLLHAIEVPLGVISVFKYAVANFDRRLSSTIYLVGFQIASSVGIVALSLPVGMLFDAAGYRTVFYVISGIVFLMLLFGAVFLSGRRDGARPEAAEMGAVG

SEQ ID NO: 50 - protein, 416 aa, Pluralibacter gergoviae strain SF84F43
MTLNIPLRNPCYRFASGYSFLFFISWSLWWSLYAIWLKGSLGLSGAALGTLYSVNQFTSMLFMVVYGVVQDRLGLNKRLIWGMGAILLLTGPFIIYLYEPLLKSHFTLGLLLGSVFFGLGYLAGNGLLDSFIEKMSRRWDFEYGTARAWGSFGYAIGALCAGVLFSINPHINFWLVSLFGALFMLINLRFRPDQATARCAEVAKVSRTDFVAVFKDANFWILVLFIIGSWSFYTIYDQQLFPVFYAGLFDSPALGARVYGYLNAFQVVLEALCMAVIPFLINRIGPKNGLLLSVAIMMLRIFSCAVFSDPWVISLVKLLHAIEVPLGVISVFKYAVANFDRRLSSTIYLVGFQIASSVGIVALSLPVGMLFDAAGYRTVFYVISGIVFLMLLFGAAFLSGRRDGARPEAAEMGAVG

SEQ ID NO: 51 - protein, 416 aa, Pluralibacter gergoviae strain JS81F13
MTLNIPLRNPCYRFASGYSFLFFISWSLWWSLYAIWLKGSLGLSGAALGTLYSVNQFTSMLFMVVYGVVQDRLGLNKRLIWGMGAILLLTGPFIIYLYEPLLKSHFTAGLLLGSVFFGLGYLAGNGLLDSFIEKMSRRWDFEYGTARAWGSFGYAIGALCAGVLFSINPHINFWLVSLFGALFMLINLHFRPDQATARCAEAAKVSRTDFAAVFKDANFWILVLFIIGSWSFYTIYDQQLFPVFYAGLFDSPALGARVYGYLNAFQVVLEALCMAVIPFLINRIGPKNGLLLSVAIMMLRIFSCAVFSDPWIISLVKLLHAIEVPLGVISVFKYAVANFDRRLSSTIYLVGFQIASSVGIVALSLPVGMLFDAAGYRTVFYVISGIVFLMLLFGAAFLSGRRDGAQPEAAEMGAVG

SEQ ID NO: 52 - protein, 416 aa, Pluralibacter gergoviae strain DL84A27
MTLNIPLRNPCYRFASGYSFLFFISWSLWWSLYAIWLKGSLGLSGAALGTLYSVNQFTSMLFMVVYGVVQDRLGLNKRLIWGMGAILLLTGPFIIYLYEPLLKSHFTAGLLLGSVFFGLGYLAGNGLLDSFIEKMSRRWDFEYGTARAWGSFGYAIGALCAGVLFSINPHINFWLVSLFGALFMLINLRFRPDQATARCAEAAKVSRTDFAAVFKDANFWILVLFIIGSWSFYTIYDQQLFPVFYAGLFDSPALGARVYGYLNAFQVVLEALCMAVIPFLINRIGPKNGLLLSVAIMMLRIFSCAVFNDPWIISLVKLLHAIEVPLGVISVFKYAVANFDRRLSSTIYLVGFQIASSVGIVALSLPVGMLFDAAGYRTVFYVISGIVFLMLLFGAAFLSGRRDGARPEAAEMGAVG

SEQ ID NO: 53 - protein, 416 aa, Pluralibacter gergoviae strain DS82E24
MTLNIPLRNPCYRFASGYSFLFFISWSLWWSLYAIWLKGSLGLSGAALGTLYSVNQFTSMLFMVVYGVVQDRLGLNKRLIWGMGAILLLTGPFIIYLYEPLLKSHFTAGLLLGSVFFGLGYLAGNGLLDSFIEKMSRRWDFEYGTARAWGSFGYAIGALCAGVLFSINPHINFWLVSLFGALFMLINLRFRPDQATARCAEAAKVSRTDFAAVFKDANFWILVLFIIGSWSFYTIYDQQLFPVFYAGLFDSPALGARVYGYLNAFQVVLEALCMAVIPFLINRIGPKNGLLLSVAIMMLRIFSCAVFSDPWIISLVKLLHAIEVPLGVISVFKYAVANFDRRLSSTIYLVGFQIASSVGIVALSLPVGMLFDAAGYRTVFYVISGIVFLMLLFGAAFLSGRRDGAQPEAAEMGAVG

SEQ ID NO: 54 - protein, 416 aa, Pluralibacter gergoviae strain SN86A38
MTLNIPLRNPCYRFASGYSFLFFISWSLWWSLYAIWLKGSLGLSGAALGTLYSVNQFTSMLFMVVYGVVQDRLGLNKRLIWGMGAILLLTGPFIIYLYEPLLKSHFTAGLLLGSVFFGLGYLAGNGLLDSFIEKMSRRWDFEYGTARAWGSFGYAIGALCAGVLFSINPHINFWLVSLFGALFMLINLRFRPDQATARCAEAAKVSRTDFAAVFKDANFWILVLFIIGSWSFYTIYDQQLFPVFYAGLFDSPALGARVYGYLNAFQVVLEALCMAVIPFLINRIGPKNGLLLSVAIMMLRIFSCAVFSDPWIISLVKLLHAIEVPLGVISVFKYAVANFDRRLSSTIYLVGFQIASSVGIVALSLPVGMLFDAAGYRTVFYVISGIVFLMLLFGAAFLSGRRDGARPEAAEMGAVG

SEQ ID NO: 55 - protein, 416 aa, Pluralibacter gergoviae strain FDAARGOS_386
MTLNIPLRNPCYRFASGYSFLFFISWSLWWSLYAIWLKGSLGLSGAALGTLYSVNQFTSMLFMVVYGVVQDRLGLNKRLIWGMGAILLLTGPFIIYLYEPLLKSHFTAGLLLGSVFFGLGYLAGNGLLDSFIEKMSRRWDFEYGTARAWGSFGYAIGALCAGVLFSINPHINFWLVSLFGALFMLINLRFRPDQATARCTEAAKVSRTDFAAVFKDANFWILVLFIIGSWSFYTIYDQQLFPVFYAGLFDSPALGARVYGYLNAFQVVLEALCMAVIPFLINRIGPKNGLLLSVAIMMLRIFSCAVFSDPWIISLVKLLHAIEVPLGVISVFKYAVANFDRRLSSTIYLVGFQIASSVGIVALSLPVGMLFDAAGYRTVFYVISGIVFLMLLFGAAFLSGRRDGARPEAAEMGAVG

SEQ ID NO: 56 - protein, 415 aa, Escherichia coli PNUSAE004051
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMCFKDKDHQCVAADAGGVKKEDFIAVFKDRNFWVFVIFIVGTWSSYNIFDQQLFPVFYAGLFESHDVGTRLYGYLNSFQVVLEALCMAIIPFFVNRVGPKNALLIGVVIMALRILSCALFVNPWIISLVKLLHAIEVPLGVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 57 - protein, 415 aa, Escherichia coli 20OR01CB08-EC
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYAGLFESHDVGTRLYGYLNSFQVVLEALCMAIIPFFVNRVGPKNALLIGVVIMALRILSCALFVNPWIISLVKLLHAIEVPLGVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 58 - protein, 253 aa, Escherichia coli O165H25 str. 2010C-4874
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMCFKDKDHQCVAADAGGVKKRILSQFSRIETSGFSSYLLWGRGLSITFLINNFFLSFMQVYSNHTM

SEQ ID NO: 59 - protein, 253 aa, Escherichia coli DEC2C
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSAGLILGALFFGLGYLAGCGLLDSFTEKMARNFHFEYGTARAWGSFGYAIGAFFAGIFFSISPHINFWLVSLFGAVFMMINMRFKDKDHQCIAADAGGVKKRILSQFSRIETSGFSSYLLWGRGLSITFLINNSFLSFMQVYSNHTM

SEQ ID NO: 60 - protein, 253 aa, Escherichia coli strain NCTC7927
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKGHQCVVVDAGGVKKRILSQFSRIETSGFSSYLLWGRGLSITFLINNSFLSFMQVYSNHTM

SEQ ID NO: 61 - protein, 253 aa, Escherichia coli KS-P039
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKRILSQFSRIETSGFSSYLLWGRGLSITFLINNFFLSFIQVYSNHTM

SEQ ID NO: 62 - protein, 253 aa, Escherichia coli DEC12B
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKRILSQFSRIETSGFSSYLLWGRGLSITFLINNFFLSFMQVYSNHTM

SEQ ID NO: 63 - protein, 301 aa, Zymomonas mobilis
MKNDKKIYGCIEGGGTKFMLALIDSDRKMLAVERVPTTTPEETLGKSVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 64 - protein, 504 aa, Bifidobacterium adolescentis [sequence omitted]

SEQ ID NO: 65 - protein, 299 aa, Helicobacter pylori UA1234
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 66 - protein, 311 aa, Helicobacter sp. MIT 01-6242
MFSVRLMGGLGNQMFIYAFAKAIKAQGYPVRLFYYDTDYNVPQTHNIRNLEIVDFGIAMCIETMCYEEPQIKKSFFERALGFIKRKLKIHSPHSSSLISDHCEIALTKDFLDTLNPNAMFNGYFQNVVFFDHLRESLLRDFTLKRPLTPANEALKHQILQTPNSCFLHIRRGDYLQIPIYVKLGSTYYNNAIKALKDKISKPHIFVFSNDIAWCKEFFLDSLDPLVIENVTFSFIENNDEGNAIEEMELMRSCQHAIIANSTFSWWAAYLIDSAQKLCIMPKHFFNDPQQEVAHKLIPPPLHSLSQTIVIG

SEQ ID NO: 67 - protein, 297 aa, Porphyromonas catoniae ATCC 51270
MKRIYLSIYGGLGNQLYILAYADYLQRMLGTRPYLLNELQRTKADTSSLDRTRRDLFSELIAYLGFQFVDTDSREFRLLKKWERHIKHYQEEPNKHGIYLQNILPPLQENHRWTLPLVCRVSGYFQSCHYVGTDFRMRVSKFLERHATSADLVREYTSMIQPEDVAIHLRRGDFVALQHTGIQLFGAEHYTKGLALQAQQQPIGRVFVFSDDFEAIGEELSLLANNYQLVLVKGLTPLQDLFLLTCFRRYVLANSTFSWWGALCSKYGDAVKVVVPKKPLLISYPEDSYFPPSWEQI

SEQ ID NO: 68 - protein, 261 aa, Akkermansia muciniphila
MNMERKTGLMNKKYVSPCFLPGMRLGNIMFTLAAACAHARTVGVECRVPWAYNDASLMLRSRLGGWVLPSTPCGTNEPPSWQEPSFAYCPVPSRIRTGGLRGYFQSARYFEGQEAFIRALFAPLTAEKEPGAVGIHIRLGDYRRLRDKHRILDPGFLRRAAGHLSSGKNRLVLFSDEPDEAAEMLARVPAFGRFALEIDRGAPCESLRRMTAMEELVMSCSSFSWWGAWLGNTRKVIVPRDWFVGGVEDYRDIYLPHWVTL

SEQ ID NO: 69 - protein, 329 aa, Basilea psittacipulmonis JF4266 DSM 24701
MSVLKKLVRTLKKKKDIPSENQEDIKPQEFGHIKHYHFWPLSNETFFNQFAQEKNLDLSQTALISCFGELSAIPKIPERYKVFFTGENIYHPDRISYSDPELYRMVDLYLGFEYRTEPKYLRFPLWVWYLCGLTKKPHFSHESIAEFIRKMNQPEFRLQSSRNRFCSHISSHDTNGIRKRMIDLILPIASVDCAGKFMNNTDELKAKFNDDKIDYLKQYRFNLCPENSESVGYITEKIFESIMAGCIPIYWGGVKQLFVEPDILNPEAFIYYEKGKEEQLAKQVEELWISPKRYEEFAAIAPFKEDAAEVIYTWIEELEKRLRAFEPKA

SEQ ID NO: 70 - protein, 325 aa, Azospirillum oryzae A2P
MIDQRTSDFLSEFLASSHRDPARLDSFLLHGPGRGARAAKPRLKIAFFDFWPEFDPAANFFVDILSARFDVSVVDNDSDLAIVSVFGTRHREARTARSMFFTGENVRPPLDGVDMSVSFDRIDDPRHYRLPLYVMHAWDHRREGATPHFCQSVLPPVPPTREEAAKRKFCAFLYKNPNCARRNDFFQMLCARRHVESVGWLLNNTGSVVKMGWLPKIRVFSRYRFAFAFENASHPGYLTEKILDAFQAGAVPLYWGDPGVLRDVAAGSFIDVSRYASDEEACDAILAADDDYDTYRRYRSTPPFLGAEDFYFDAFRLAEWIESRL

SEQ ID NO: 71 - protein, 264 aa, Escherichia coli
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 72 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 73 - protein, 456 aa, Escherichia coli
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 74 - protein, 478 aa, Escherichia coli
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 75 - protein, 373 aa, Escherichia coli [sequence omitted]

SEQ ID NO: 76 - protein, 321 aa, Escherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 77 - protein, 438 aa, Escherichia coli
MGNTSIQTQSYRAVDKDAGQSRSYIIPFALLCSLFFLWAVANNLNDILLPQFQQAFTLTNFQAGLIQSAFYFGYFIIPIPAGILMKKLSYKAGIITGLFLYALGAALFWPAAEIMNYTLFLVGLFIIAAGLGCLETAANPFVTVLGPESSGHFRLNLAQTFNSFGAIIAVVFGQSLILSNVPHQSQDVLDKMSPEQLSAYKHSLVLSVQTPYMIIVAIVLLVALLIMLTKFPALQSDNHSDAKQGSFSASLSRLARIRHWRWAVLAQFCYVGAQTACWSYLIRYAVEEIPGMTAGFAANYLTGTMVCFFIGRFTGTWLISRFAPHKVLAAYALIAMALCLISAFAGGHVGLIALTLCSAFMSIQYPTIFSLGIKNLGQDTKYGSSFIVMTIIGGGIVTPVMGFVSDAAGNIPTAELIPALCFAVIFIFARFRSQTATN

SEQ ID NO: 78 - protein, 949 aa, Bacteroides fragilis NCTC 9343
MQKLLSLPSNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAVSVFPMVNSVEELGLVLRWMLSEPELEEGKNIWLRSEHFSADEISAGANLKRLYAQREEFRKGNWKALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 79 - protein, 417 aa, Escherichia coli [sequence omitted]

SEQ ID NO: 80 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 81 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 82 - protein, 349 aa, Neisseria meningitidis
MQNNNEFKIGNRSVGYNHEPLIICEIGINHEGSLKTAFEMVDAAYNAGAEVVKHQTHIVEDEMSDEAKQVIPGNADVSIYEIMERCALNEEDEIKLKEYVESKGMIFISTPFSRAAALRLQRMDIPAYKIGSGECNNYPLIKLVASFGKPIILSTGMNSIESIKKSVEIIREAGVPYALLHCTNIYPTPYEDVRLGGMNDLSEAFPDAIIGLSDHTLDNYACLGAVALGGSILERHFTDRMDRPGPDIVCSMNPDTFKELKQGAHALKLARGGKKDTIIAGEKPTKDFAFASVVADKDIKKGELLSGDNLWVKRPGNGDFSVNEYETLFGKVAACNIRKGAQIKKTDIE

SEQ ID NO: 83 - protein, 609 aa, Escherichia coli
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 84 - protein, 188 aa, Escherichia coli
MYERYAGLIFDMDGTILDTEPTHRKAWREVLGHYGLQYDIQAMIALNGSPTWRIAQAIIELNQADLDPHALAREKTEAVRSMLLDSVEPLPLVDVVKSWHGRRPMAVGTGSESAIAEALLAHLGLRHYFDAVVAADHVKHHKPAPDTFLLCAQRMGVQPTQCVVFEDADFGIQAARAAGMDAVDVRLL

SEQ ID NO: 85 - protein, 373 aa, Campylobacter jejuni
MVKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 86 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

SEQ ID NO: 87 - protein, 268 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLG

SEQ ID NO: 88 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 89 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 90 - protein, 498 aa, Photobacterium sp. JT-ISH-224
MSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 91 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 92 - protein, 265 aa, Escherichia coli O55H7
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 93 - protein, 275 aa, Neisseria meningitidis MC58
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 94 - protein, 338 aa, Escherichia coli
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 95 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 96 - protein, 445 aa, Escherichia coli
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 97 - protein, 456 aa, Escherichia coli
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 98 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLCKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 99 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLFKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 100 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLAVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 101 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILYDHAGYQTVFFAISGIVCLMLLFGIFFLSKKREQIVMETPVPSAI

SEQ ID NO: 102 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILEDHAGYQTVFFAISGIVCLMLLFGIFFLSKKREQIVMETPVPSAI

SEQ ID NO: 103 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILQDHAGYQTVFFAISGIVCLMLLFGIFFLSKKREQIVMETPVPSAI

SEQ ID NO: 104 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILLDHAGYQTVFFAISGIVCLMLLFGIFFLSKKREQIVMETPVPSAI

SEQ ID NO: 105 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILADHAGYQTVFFAISGIVCLMLLFGIFFLSKKREQIVMETPVPSAI

SEQ ID NO: 106 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILTDHAGYQTVFFAISGIVCLMLLFGIFFLSKKREQIVMETPVPSAI

SEQ ID NO: 107 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILCDHAGYQTVFFAISGIVCLMLLFGIFFLSKKREQIVMETPVPSAI

SEQ ID NO: 108 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILSDHAGYQTVFFAISGIVCLMLLFGIFFLSKKREQIVMETPVPSAI

SEQ ID NO: 109 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILIDHAGYQTVFFAISGIVCLMLLFGIFFLSKKREQIVMETPVPSAI

SEQ ID NO: 110 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLIKLLHAIEVPLAVISVFKYSVANFDKRLSSTIFLIGFQIASSLGIVLLSTPTGILTDHAGYQTVFFAISGIVCLMLLFGIFFLSKKREQIVMETPVPSAI

SEQ ID NO: 111 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLMKLLHAIEVPLGVISVFKYSVANFDKRLSSTIFLIGFQIASSLGIVLLSTPTGILGDHAGYQTVFFAISGIVCLMLLFGIFFLSKKREQIVMETPVPSAI

SEQ ID NO: 112 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLLKLLHAIEVPLGVISVFKYSVANFDKRLSSTIFLIGFQIASSLGIVLLSTPTGILSDHAGYQTVFFAISGIVCLMLLFGIFFLSKKREQIVMETPVPSAI

SEQ ID NO: 113 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLFKLLHAIEVPLGVISVFKYSVANFDKRLSSTIFLIGFQIASSLGIVLLSTPTGILVDHAGYQTVFFAISGIVCLMLLFGIFFLSKKREQIVMETPVPSAI

SEQ ID NO: 114 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLIKLLHAIEVPLGVISVFKYSVANFDKRLSSTIFLIGFQIASSLGIVLLSTPTGILCDHAGYQTVFFAISGIVCLMLLFGIFFLSKKREQIVMETPVPSAI

SEQ ID NO: 115 - protein, 403 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKRE

SEQ ID NO: 116 - protein, 408 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVME

SEQ ID NO: 117 - protein, 403 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLGVISVFKYSVANFDKRLSSTIFLIGFQIASSLGIVLLSTPTGILVDHAGYQTVFFAISGIVCLMLLFGIFFLSKKRE

SEQ ID NO: 118 - protein, 408 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLGVISVFKYSVANFDKRLSSTIFLIGFQIASSLGIVLLSTPTGILVDHAGYQTVFFAISGIVCLMLLFGIFFLSKKREQIVME

SEQ ID NO: 119 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILGDHAGYQTVFFAISGIVCLMLLFGIFFLSKKREQIVMETPVPSAI

SEQ ID NO: 120 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILMDHAGYQTVFFAISGIVCLMLLFGIFFLSKKREQIVMETPVPSAI

SEQ ID NO: 121 - protein, 415 aa, Artificial sequence
  cscB variant
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILNDHAGYQTVFFAISGIVCLMLLFGIFFLSKKREQIVMETPVPSAI